(19) United States
(12) Patent Application Publication
(10) Pub. No.: US 2025/0295755 A1
(43) Pub. Date: Sep. 25, 2025

(54) MRNA VACCINE COMPOSITION

(71) Applicant: Sail Biomedicines, Inc., Cambridge, MA (US)

(72) Inventors: Munir MOSAHEB, Cambridge, MA (US); Siddharth PATEL, Cambridge, MA (US); Emad ARAFA, Cambridge, MA (US); Roman BOGORAD, Cambridge, MA (US)

(21) Appl. No.: 18/703,109

(22) PCT Filed: Oct. 19, 2022
(86) PCT No.: PCT/US2022/047107

§ 371 Date: Apr. 19, 2024

Related U.S. Application Data
(60) Provisional application No. 63/270,964, filed on Oct. 22, 2021.
(60) Provisional application No. 63/290,889, filed on Dec. 17, 2021.
(60) Provisional application No. 63/320,647, filed on Mar. 16, 2022.

(51) Int. Cl. A61K 39/215; A61K 9/1272; A61K 9/1277; A61K 9/19; A61K 9/51; A61K 39/00; A61K 47/18; A61K 47/22; A61P 31/14; B82Y 5/00; C12N 7/00

(52) U.S. Cl. CPC A61K 39/215; A61K 9/1272; A61K 9/1277; A61K 9/19; A61K 9/5123; A61K 9/5176; A61K 9/5192; A61K 47/18; A61K 47/22; A61P 31/14; C12N 7/00; A61K 2039/55555; B82Y 5/00; C12N 2770/20034

(57) ABSTRACT

Disclosed herein are nucleic acid vaccine compositions including one or more polynucleotides encoding one or more antigenic polypeptide, formulated within a lipid reconstructed plant messenger packs (LPMPs) comprising natural lipids and an ionizable lipid. The disclosure also includes a method for making a nucleic acid vaccine, comprising reconstituting a film comprising purified PMP lipids in the presence of an ionizable lipid to produce a LPMP comprising the ionizable lipid, and loading into the LPMPs with one or more polynucleotides encoding one or more antigenic polypeptides.

### CROSS-REFERENCE TO RELATED APPLICATIONS

This application claims benefit of priority to U.S. Provisional Application No. 63/270,964 filed Oct. 22, 2021; U.S. Provisional Application No. 63/290,889 filed Dec. 17, 2021; and U.S. Provisional Application No. 63/320,647 filed Mar. 16, 2022; all of which are herein incorporated by reference in their entirety.

### BACKGROUND

Newly emerging acute respiratory virus infections caused by novel coronavirus is a significant public health concern. The pandemic disease that the SARSCoV-2 virus causes has been named by the World Health Organization (WHO) as COVID-19 (Coronavirus Disease 2019). The public health crisis caused by SARS-CoV-2 reinforces the importance of rapidly developing effective, easily scalable, and stable vaccine delivery against these viruses.

RNA vaccines have recently been showing great promise. A need therefore exists for developing an enhanced RNA delivery system for a more effective, easily scalable, and stable vaccine delivery.

### SUMMARY OF THE INVENTION

In one aspect, provided herein is a nucleic acid vaccine, comprising one or more polynucleotides encoding one or more antigenic polypeptides derived from an infectious agent that causes an infectious disease, disorder, or condition. The one or more polynucleotides are formulated within a lipid reconstructed plant messenger packs (LPMPs) comprising natural lipids and an ionizable lipid. The ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

In another aspect, provided herein is a method for making a nucleic acid vaccine. The method comprises reconstituting a film comprising purified PMP lipids in the presence of an ionizable lipid to produce a lipid reconstructed plant messenger packs (LPMP) comprising the ionizable lipid.

The ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

The method further comprises loading into the LPMPs with one or more polynucleotides encoding one or more antigenic polypeptides derived from an infectious agent that causes an infectious disease, disorder, or condition.

In some embodiments, the polynucleotides are polynucleotide constructs, which encode one or more wild type or engineered antigens (or an antibody to an antigen). The antigen may be derived from an infectious agent. In some embodiments, the infectious agent is a virus, e.g., a virus selected from the group consisting of: an influenza virus, a corona virus, a mosquito-borne virus, a hepatitis virus, and an HIV virus. In some embodiments, the infectious agent is a virus, e.g., a respiratory syncytial virus, a rhinovirus, an adenovirus, or a parainfluenza virus. For instance, the infectious agent may be one or more strains of the viruses.

In some embodiments, the antigenic polypeptide encoded by the polynucleotide is a corona virus, or a fragment or subunit thereof. In some embodiments, the antigenic polypeptide is spike protein (S) of a MERS virus (MERS-CoV), a SARS virus (SARS-CoV), or a fragment or subunit thereof.

In some embodiments, the antigenic polypeptide is a SARS virus, or a fragment or subunit thereof. The antigenic polypeptide may be a SARS-CoV-2 spike protein or a SARS-CoV-2 spike glycoprotein. In one embodiment, the antigenic polypeptide is a wild-type SARS-CoV-2 spike glycoprotein.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA.

In some embodiments, the mRNA is derived from (a) a DNA molecule, or (b) an RNA molecule. In the mRNA, T is optionally substituted with U.

In some embodiments, the mRNA is derived from a DNA molecule. The DNA molecule can further comprise a promoter. In some embodiments, the promoter is a T7 promoter, a T3 promoter, or an SP6 promoter. In some embodiments, the promoter is located at the 5′ UTR.

In some embodiments, the mRNA is an RNA molecule. The RNA molecule may be a self-replicating RNA molecule.

In some embodiments, the mRNA is an RNA molecule. The RNA molecule may further comprise a 5′ cap. The 5′ cap can have a Cap 1 structure, a Cap 1 (m6A) structure, a Cap 2 structure, a Cap 3 structure, a Cap 0 structure, or any combination thereof.

In some embodiments, the mRNA comprises an open reading frame (ORF) that encodes a SARS-CoV-2 spike (S) glycoprotein having a double proline stabilizing mutation. In one embodiment, the double proline stabilizing mutation is at positions corresponding to K986 and V987 of a wild-type SARS-CoV-2 S glycoprotein.

In some embodiments, the mRNA comprises a 5′ untranslated region (UTR) and/or a 3′ UTR.

In some embodiments, the mRNA comprises a 5′ UTR. The 5′ UTR may comprise a Kozak sequence.

In some embodiments, the mRNA comprises a 3′ UTR. In some embodiments, the 3′ UTR comprises one or more sequences derived from an amino-terminal enhancer of split (AES). In some embodiments, the 3′ UTR comprises a sequence derived from mitochondrially encoded 12S rRNA (mtRNRI).

In some embodiments, the mRNA comprises a poly(A) sequence. In one embodiment, the poly(A) sequence is a 110-nucleotide sequence consisting of a sequence of 30 adenosine residues, a 10-nucleotide linker sequence, and a sequence of 70 adenosine residues.

In some embodiments, the polynucleotide is encapsulated by the lipid reconstructed plant messenger packs (LPMPs). In some embodiments, the polynucleotide is embedded on the surface of the LPMPs. In some embodiments, the polynucleotide is conjugated to the surface of the LPMPs.

In some embodiments, the LPMP is produced by a method comprising lipid extrusion. In some embodiments, the LPMP is produced by a method comprising processing a solution comprising a lipid extract of the PMPs in a microfluidics device comprising an aqueous phase, thereby producing the LPMPs. In some embodiments, the aqueous phase comprises the polynucleotides.

In some embodiments, the natural lipids of the LPMPs are extracted from lemon or algae.

In some embodiments, the ionizable lipid of the LPMPs is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

In one embodiment, the ionizable lipid is C12-200.

In some embodiments, the ionizable lipid is

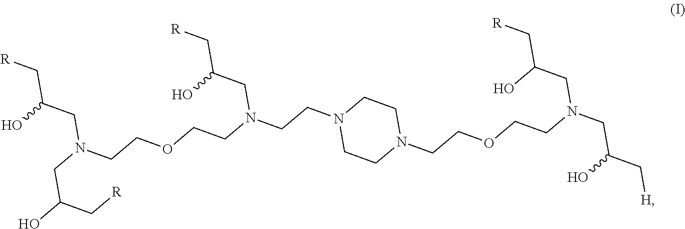

wherein R is C8-C14 alkyl group.

In some embodiments, the reconstitution is performed in the presence of a sterol, thereby producing a LPMP that comprises natural lipids, a ionizable lipid, and a sterol. In some embodiments, the sterol is cholesterol or sitosterol.

In some embodiments, the reconstitution is performed in the presence of a PEGylated lipid (or a PEG-lipid conjugate), thereby producing a LPMP that comprises natural lipids, a ionizable lipid, and a PEG-lipid conjugate. In some embodiments, the PEG-lipid conjugate is C14-PEG2k, C18-PEG2k, or DMPE-PEG2k. In some embodiments, the PEG-lipid conjugate is PEG-DMG or PEG-PE. In some embodiments, the PEG-DMG is PEG2000-DMG or PEG2000-PE.

In some embodiments, the LPMPs further comprise a sterol and a polyethylene glycol (PEG)-lipid conjugate.

In some embodiments, the LPMP comprises:
about 20 mol % to about 50 mol % of the ionizable lipid,
about 20 mol % to about 60 mol % of the natural lipids,
about 7 mol % to about 20 mol % of the sterol, and
about 0.5 mol % to about 3 mol % of the polyethylene glycol (PEG)-lipid conjugate.

In some embodiments, the LPMP comprises:
about 35 mol % of the ionizable lipid,
about 50 mol % of the natural lipids,
about 12.5 mol % of the sterol, and
about 2.5 mol % the polyethylene glycol (PEG)-lipid conjugate.

In one embodiment, the LPMPs comprise the ionizable lipid:natural lipids:sterol:PEG-lipid at a molar ratio of about 35:50:12.5:2.5. In one embodiment, the LPMPs comprise the ionizable lipid:natural lipids:sterol:PEG-lipid at a molar ratio of about 35:20:42.5:2.5.

In some embodiments, the LPMPs comprise:
natural lipids extracted from lemon or algae,
C12-200,
cholesterol, and
DMPE-PEG2k.

In one embodiment, the LPMPs comprise:
natural lipids extracted from lemon,
C12-200,
cholesterol, and
DMPE-PEG2k. The LPMPs may comprise C12-200:lemon lipids:cholesterol:DMPE-PEG2k at a molar ratio of about 35:50:12.5:2.5.

In one embodiment, the LPMPs comprise:
natural lipids extracted from algae,
C12-200,
cholesterol, and
DMPE-PEG2k. The LPMPs may comprise C12-200:algae lipids:cholesterol:DMPE-PEG2k at a molar ratio of about 35:20:42.5:2.5.

In some embodiments, the LPMP is a lipophilic moiety selected from the group consisting of a lipoplex, a liposome, a lipid nanoparticle, a polymer-based carrier, an exosome, a lamellar body, a micelle, and an emulsion. In one embodiment, the LPMP is a liposome selected from the group consisting of a cationic liposome, a nanoliposome, a proteoliposome, a unilamellar liposome, a multilamellar liposome, a ceramide-containing nanoliposome, and a multivesicular liposome. In one embodiment, the LPMP is a lipid nanoparticle.

In some embodiments, the LPMP has a size of less than about 200 nm. In one embodiment, the LPMP has a size of less than about 150 nm. In one embodiment, the LPMP has a size of less than about 100 nm. In one embodiment, the LPMP has a size of about 55 nm to about 80 nm.

In some embodiments, the nucleic acid vaccine has a total lipid:polynucleotide weight ratio of about 50:1 to about 10:1. In one embodiment, the nucleic acid vaccine has a total lipid:polynucleotide weight ratio of about 44:1 to about 24:1. In one embodiment, the nucleic acid vaccine has a total lipid:polynucleotide weight ratio of about 40:1 to about 28:1. In one embodiment, the nucleic acid vaccine has a total lipid:polynucleotide weight ratio of about 38:1 to about 30:1. In one embodiment, the nucleic acid vaccine has a total lipid:polynucleotide weight ratio of about 37:1 to about 33:1.

In some embodiments, the nucleic acid vaccine, e.g., the aqueous phase, further comprises a HEPES or TRIS buffer. The HEPES or TRIS buffer may have a pH of about 7.0 to about 8.5. The HEPES or TRIS buffer can be at a concentration of about 7 mg/mL to about 15 mg/mL. The aqueous phase may further comprise about 2.0 mg/mL to about 4.0 mg/mL of NaCl.

In some embodiments, the nucleic acid vaccine, e.g., the aqueous phase comprises water, PBS, or a citrate buffer. In one embodiment, the aqueous phase comprises a citrate buffer having a pH of about 3.2.

In some embodiments, the aqueous phase and the lipid solution are mixed at a 3:1 volumetric ratio.

In some embodiments, the nucleic acid vaccine further comprises one or more cryoprotectants. The one or more cryoprotectants may be sucrose, glycerol, or a combination thereof. In one embodiment, the nucleic acid vaccine comprises a combination of sucrose at a concentration of about 70 mg/mL to about 110 mg/mL and glycerol at a concentration of about 50 mg/mL to about 70 mg/mL.

In some embodiments, the nucleic acid vaccine is a lyophilized composition. The lyophilized nucleic acid vaccine may comprise one or more lyoprotectants. The lyophilized nucleic acid vaccine may comprise a poloxamer, potassium sorbate, sucrose, or any combination thereof. In one embodiment, the lyophilized nucleic acid vaccine comprises a poloxamer, e.g., poloxamer 188.

In some embodiments, the nucleic acid vaccine is a lyophilized composition. In one embodiment, the lyophilized nucleic acid vaccine comprises about 0.01 to about 1.0% w/w of the polynucleotides. In one embodiment, the lyophilized nucleic acid vaccine comprises about 1.0 to about 5.0% w/w lipids. In one embodiment, the lyophilized nucleic acid vaccine comprises about 0.5 to about 2.5% w/w of TRIS buffer. In one embodiment, the lyophilized nucleic acid vaccine comprises about 0.75 to about 2.75% w/w of NaCl. In one embodiment, the lyophilized nucleic acid vaccine comprises about 85 to about 95% w/w of a sugar, e.g., sucrose. In one embodiment, the lyophilized nucleic acid vaccine comprises about 0.01 to about 1.0% w/w of a poloxamer, e.g., poloxamer 188. In one embodiment, the lyophilized nucleic acid vaccine comprises about 1.0 to about 5.0% w/w of potassium sorbate.

Aspects of the invention also provide for methods of preventing or reducing the transmission of an infectious disease, disorder, or condition. The method comprises administering to a subject the nucleic acid vaccine described in the above aspects of the invention, thereby preventing or reducing the transmission of an infectious disease, disorder, or condition. In some embodiments, the method reduces the transmission of an infectious disease, disorder, or condition by at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%). Alternatively, the method comprises administering to a subject the nucleic acid vaccine described in the above aspects of the invention, thereby reducing the transmission level of an infectious disease, disorder, or condition to another subject, to less than 10% (e.g., less than 5%, less than 4%, less than 3%, less than 2%, less than 1%, less than 0.5%, less than 0.4%, less than 0.3%, less than 0.2%, or less than 0.1%).

### Definitions

As used herein, the term “effective amount,” “effective concentration,” or “concentration effective to” refers to an amount of a LPMP, or nucleic acid composition, sufficient to effect the recited result or to reach a target level (e.g., a predetermined or threshold level) in or on a target organism.

As used herein, the term “therapeutic agent” refers to an agent that can act on an animal, e.g., a mammal (e.g., a human), an animal pathogen, or a pathogen vector, such as an antifungal agent, an antibacterial agent, a virucidal agent, an anti-viral agent, an insecticidal agent, a nematicidal agent, an antiparasitic agent, or an insect repellent.

As defined herein, the term “nucleic acid” and “polynucleotide” are interchangeable and refer to RNA or DNA that is linear or branched, single or double stranded, or a hybrid thereof, regardless of length (e.g., at least 2, 3, 4, 5, 6, 7, 8, 9, 10, 20, 30, 40, 50, 100, 150, 200, 250, 500, 1000, or more nucleic acids). The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, or more amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, sap (e.g., xylem sap and phloem sap), and various forms of cells and culture (e.g., single cells, protoplasts, embryos, and callus tissue). The plant tissue may be in a plant or in a plant organ, tissue, or cell culture.

As used herein, the term “modified PMPs” or “modified LPMPs” refers to a composition including a plurality of PMPs or LPMPs that include one or more heterologous agents (e.g., one or more exogenous lipids, such as a ionizable lipids, e.g., a PMP or LPMP comprising an ionizable lipid and a sterol and/or a PEGylated lipid) capable of increasing cell uptake (e.g., animal cell uptake, plant cell uptake, bacterial cell uptake, or fungal cell uptake) of the PMP or LPMP, or a portion or component thereof, relative to an unmodified PMP or LPMP; capable of enabling or increasing delivery of a heterologous functional agent (e.g., an agricultural or therapeutic agent) by the PMP or LPMP to a cell, and/or capable of enabling or increasing loading (e.g., loading efficiency or loading capacity) of a heterologous functional agent (e.g., an agricultural or therapeutic agent). The PMPs or LPMPs may be modified in vitro or in vivo.

As used herein, the term “unmodified PMPs” or “unmodified LPMPs” refers to a composition including a plurality of PMPs or LPMPs that lack a heterologous cell uptake agent capable of increasing cell uptake (e.g., animal cell uptake, plant cell uptake, bacterial cell uptake, or fungal cell uptake) of the PMP.

As used herein, the term “cell uptake” refers to uptake of a PMP or LPMP or a portion or component thereof (e.g., a polynucleotide carried by the PMP or LPMP) by a cell, such as an animal cell, a plant cell, bacterial cell, or fungal cell. For example, uptake can involve transfer of the PMP (e.g., LPMP) or a portion of component thereof from the extracellular environment into or across the cell membrane, the cell wall, the extracellular matrix, or into the intracellular environment of the cell). Cell uptake of PMPs (e.g., LPMPs) may occur via active or passive cellular mechanisms. Cell uptake includes aspects in which the entire PMP (e.g., LPMP) is taken up by a cell, e.g., taken up by endocytosis. In some embodiments, one or more polynucleotides are exposed to the cytoplasm of the target cell following endocytosis and endosomal escape. In some embodiments, a modified LPMP (e.g., a LPMP comprising an ionizable lipid, e.g., a LPMP comprising an ioniable lipid and a sterol and/or a PEGylated lipid) has an increased rate of endosomal escape relative to an unmodified LPMP. Cell uptake also includes aspects in which the PMP (e.g., LPMP) fuses with the membrane of the target cell. In some embodiments, one or more polynucleotides are exposed to the cytoplasm of the target cell following membrane fusion. In some embodiments, a LPMPs has an increased rate of fusion with the membrane of the target cell (e.g., is more fusogenic) relative to an unmodified LPMP.

As used herein, the term “cell-penetrating agent” refers to agents that alter properties (e.g., permeability) of the cell wall, extracellular matrix, or cell membrane of a cell (e.g., an animal cell, a plant cell, a bacterial cell, or a fungal cell) in a manner that promotes increased cell uptake relative to a cell that has not been contacted with the agent.

As used herein, the term “plant extracellular vesicle”, “plant EV”, or “EV” refers to an enclosed lipid-bilayer structure naturally occurring in a plant. Optionally, the plant EV includes one or more plant EV markers. As used herein, the term “plant EV marker” refers to a component that is naturally associated with a plant, such as a plant protein, a plant nucleic acid, a plant small molecule, a plant lipid, or a combination thereof, including but not limited to any of the plant EV markers listed in the Appendix. In some instances, the plant EV marker is an identifying marker of a plant EV but is not a pesticidal agent. In some instances, the plant EV marker is an identifying marker of a plant EV and also a pesticidal agent (e.g., either associated with or encapsulated by the plurality of PMPs or LPMPs, or not directly associated with or encapsulated by the plurality of PMPs or LPMPs).

As used herein, the term “plant messenger pack” or “PMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell, the enrichment or isolation removing one or more contaminants or undesired components from the source plant. PMPs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, protein-nucleic acid aggregates, lipoprotein aggregates, or lipido-proteic structures). Preferably, a PMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m).

A lipid reconstructed PMP (LPMP) is used herein. The terms “lipid reconstructed PMP” and “LPMP” refer to a PMP that has been derived from a lipid structure (e.g., a lipid bilayer, unilamellar, multilamellar structure; e.g., a vesicular lipid structure) derived from (e.g., enriched, isolated or purified from) a plant source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LPMP, as is described herein. The method may, if desired, further comprise sonication, freeze/thaw treatment, and/or lipid extrusion, e.g., to reduce the size of the reconstituted PMPs. Alternatively, LPMPs may be produced using a microfluidic device (such as a NanoAssemblr® IGNITE™ microfluidic instrument (Precision NanoSystems)).

As used herein, the term “cationic lipid” refers to an arphiphilic molecule (e.g. a lipid or a lipidoid) that is positively charged, containing a cationic group (e.g. a cationic head group).

As used herein, the term “ionizable lipid” refers to an amphiphilic molecule (e.g., a lipid or a lipidoid, e.g., a synthetic lipid or lipidoid) containing a group (e.g., a head group) that can be ionized, e.g., dissociated to produce one or more electrically charged species, under a given condition (e.g., pH).

It has been surprisingly found that ionizable lipids comprising alkyl chains with multiple sites of unsaturation, e.g., at least two or three sites of unsaturation, are particularly useful for forming lipid particles with increased membrane fluidity. A number of ionizable lipids and related analogs, suitable for use herein, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes.

In some embodiments, ionizable lipids are ionizable such that they can dissociate to exist in a positively charged form depending on pH. The ionization of an ionizable lipid affects the surface charge of a lipid nanoparticle comprising the ionizable lipid under different pH conditions. The surface charge of the lipid nanoparticlein turn can influence its plasma protein absorption, blood clearance, and tissue distribution (Semple, S. C., et al., Adv. Drug Deliv Rev 32:3-17 (1998)) as well as its ability to form endosomolytic non-bilayer structures (Hafez, I. M., et al., Gene Ther 8: 1188-1196 (2001)) that can influence the intracellular delivery of nucleic acids.

In some embodiments, ionizable lipids are those that are generally neutral, e.g., at physiological pH (e.g., pH about 7), but can carry net charge(s) at an acidic pH or basic pH. In one embodiment, ionizable lipids are those that are generally neutral at pH about 7, but can carry net charge(s) at an acidic pH. In one embodiment, ionizable lipids are those that are generally neutral at pH about 7, but can carry net charge(s) at a basic pH.

In some embodiments, ionizable lipids do not include those cationic lipids or anionic lipids that generally carry net charge(s) at physiological pH (e.g., pH about 7).

As used herein, the term “lipidoid” refers to a molecule having one or more characteristics of a lipid.

As used herein, the term “stable LPMP formulation” refers to a LPMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of LPMPs (e.g., LPMPs per mL of solution) relative to the number of LPMPs in the LPMP formulation (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., cell wall penetrating activity and/or activity of the mRNA formulated within the LPMP) relative to the initial activity of the LPMP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

### BRIEF DESCRIPTION OF THE DRAWINGS

FIG. 1A shows the size and polydispersity of the particles of the LPMP/SARS-CoV-2 A (recPMP1, recLemon) and LPMP/SARS-CoV-2 B (recPMP2, recAlgae), as compared to those of LNP, prepared according to Example 2 (Table 2). FIG. 1B shows the encapsulation efficiency of the LPMP/SARS-CoV-2 A (recPMP1, recLemon) and LPMP/SARS-CoV-2 B (recPMP2, recAlgae), as compared to those of LNP, prepared according to Example 2 (Table 2).

FIGS. 2A-2C show the levels of cytokine IL6 (FIG. 2A), chemokine CXCL12 (FIG. 2B), and cytokine SCF (stem cell factor) (FIG. 2C) in the serum of the hamsters at 6 and 24 hours, after a single dose intramuscular vaccination of LPMP/SARS-CoV-2 vaccines in the hamsters. Two vaccine formulations were administered at D0: LPMP/SARS-CoV-2 A (recLemon LPMP, containing S mRNA 10 μg) and LPMP/SARS-CoV-2 B (recAlgae LPMP, containing S mRNA 10 μg). Control was unvaccinated hamsters with EPO mRNA administered at D0.

FIGS. 3A-3B show the levels of antibody (IgG) specific to S antigen (FIG. 3A) and S1 RBD antigen (FIG. 3B) of SARS-CoV-2 in the serum of the hamsters at 10 days, after a single dose intramuscular vaccination of LPMP/SARS-CoV-2 vaccines in the hamsters. Two vaccine formulations were administered at D0: LPMP/SARS-CoV-2 A (recLemon LPMP, containing S mRNA 10 μg) and LPMP/SARS-CoV-2 B (recAlgae LPMP, containing S mRNA 10 μg). Control was unvaccinated hamsters with EPO mRNA administered at D0.

FIG. 4 show the levels of antibody (IgG) specific to S1 RBD antigen of SARS-CoV-2 in the serum of the hamsters at 21 days, after intramuscular vaccination of LPMP/SARS-CoV-2 vaccines in the hamsters. Two types of vaccine formulations were administered at D0, at different dosages: LPMP/SARS-CoV-2 A (recLemon LPMP, containing S mRNA 10 μg, 20 μg) and LPMP/SARS-CoV-2 B (recAlgae LPMP, containing S mRNA 10 μg, 20 μg). Control was unvaccinated hamsters with EPO mRNA administered at D0.

FIG. 5 show the levels of neutralizing antibody titer against the SARS-CoV-2 original strain in the serum of the hamsters at 35 days, after intramuscular vaccination of LPMP/SARS-CoV-2 vaccines in the hamsters. Two types of vaccine formulations were administered at D0, at different dosages: LPMP/SARS-CoV-2 A (recLemon LPMP, containing S mRNA 10 μg, 20 μg) and LPMP/SARS-CoV-2 B (recAlgae LPMP, containing S mRNA 10 μg, 20 μg). Control was unvaccinated hamsters with EPO mRNA administered at D0.

FIG. 6 show the results of T cell responses against S antigen of SARS-CoV-2 in the spleens of the hamsters at 35 days, after intramuscular vaccination of LPMP/SARS-CoV-2 vaccines in the hamsters. Two types of vaccine formulations were administered at D0, at different dosages: LPMP/SARS-CoV-2 A (recLemon LPMP, containing S mRNA 10 μg, 20 μg) and LPMP/SARS-CoV-2 B (recAlgae LPMP, containing S mRNA 10 μg, 20 μg). Control was unvaccinated hamsters with EPO mRNA administered at D0.

FIGS. 7A-7D show the results of loss of body weight of the hamsters as a function of days post SARS-CoV-2 challenge (administered on D28 via intranasal dose of 10⁵ pfu TCID₅₀ per animal). Two types of vaccine formulations were administered at D0, at different dosages: LPMP/SARS-CoV-2 A (recLemon LPMP, containing S mRNA 10 μg, FIG. 7A; containing S mRNA 20 μg, FIG. 7B) and LPMP/SARS-CoV-2 B (recAlgae LPMP, containing S mRNA 10 μg, FIG. 7C; containing S mRNA 20 μg, FIG. 7D). Control was unvaccinated hamsters with EPO mRNA administered at D0.

FIG. 8 show the results of loss of body weight of the hamsters as a function of days post SARS-CoV-2 challenge (administered on D28 via intranasal dose of 10⁵ pfu TCID₅₀ per animal). LPMP/SARS-CoV-2 A (recLemon LPMP, containing S mRNA 10 μg) vaccine formulation was administered at D0. Control was unvaccinated hamsters with EPO mRNA administered at D0.

FIG. 9 show the results of infectious viral particles in the lungs of the hamsters 4 days post SARS-CoV-2 challenge (administered on D28 via intranasal dose of 10⁵ pfu TCID₅₀ per animal). LPMP/SARS-CoV-2 A (recLemon LPMP, containing S mRNA 10 μg) vaccine formulation was administered at D0. Control was unvaccinated hamsters with EPO mRNA administered at D0.

FIGS. 10A-10B show the results of viral load in the lungs (FIG. 10A) and nasal mucosa (FIG. 10B) of the hamsters 4 days post SARS-CoV-2 challenge (administered on D28 via intranasal dose of 10⁵ pfu TCID₅₀ per animal). LPMP/SARS-CoV-2 A (recLemon LPMP, containing S mRNA 10 μg) vaccine formulation was administered at D0. Control was unvaccinated hamsters with EPO mRNA administered at D0.

FIGS. 11A-11B show the results of T cell responses against S antigen of SARS-CoV-2 in the spleens of the mice (FIG. 11A) and in the Peyer's patches of the mice (FIG. 11B) at 12 days, after oral vaccination of LPMP/SARS-CoV-2 vaccines in the mice. LPMP/SARS-CoV-2 A (recLemon LPMP, containing S mRNA 200 μg) vaccine formulation was administered at D0. Control was PBS.

FIG. 12A shows the results of T cell responses against S antigen of SARS-CoV-2 in the mice at 12 days, after intranasal vaccination of LPMP/SARS-CoV-2 A (recLemon LPMP, containing S mRNA 0.1 μg, 10 μg) in the mice. FIG. 12B shows the levels of cytokine TNFα in the blood of the mice at 12 days, after intranasal vaccination of LPMP/SARS-CoV-2 A (recLemon LPMP, containing S mRNA 0.1 μg, 10 μg) in the mice. Control was PBS.

FIG. 13 shows the stability of the LPMP/mRNA formulations stored at 4° C. over a period of time (Day 1, Day 30, Day 60), being evaluated by measuring the radiance of the mice (n=2) 4 hours after the mice were intravenously administered with a dosage of LPMP/mRNA fLuciferase (containing mRNA 5 μg). LPMP/mRNA fLuciferase A (recLemon LPMP) and LPMP/mRNA fLuciferase B (recAlgae LPMP) formulation were evaluated.

FIG. 14A shows the stability of the LPMP/mRNA formulations with lyophilization and without lyophilization over a period of time (Day 1, Day 7), with the images taken of the mouse 6 hours after the mouse was intravenously administered with a dosage of LPMP/mRNA fLuciferase (containing mRNA 0.2 mg/kg). RecLemon LPMP/mRNA fLuciferase and recAlage LPMP/mRNA fLuciferase formulation were evaluated. FIG. 14B shows the stability of the LPMP/mRNA formulations with lyophilization and at 4° C., with the images taken of the mouse 6 hours after the mouse was intravenously administered with a dosage of LPMP/mRNA fLuciferase (containing mRNA 0.2 mg/kg). RecLemon LPMP/mRNA fLuciferase formulation was evaluated.

FIG. 15 shows the immune response against S antigen of SARS-CoV-2 in mice after administration of LPMP/SARS-CoV-2 formulation in the mice via intramuscular route (0.4 mg/kg), intranasal route (0.4 mg/kg), and oral route (8 mg/kg). LPMP/SARS-CoV-2 A (recLemon LPMP, containing S mRNA) vaccine formulation was administered at D0. Control was PBS.

FIGS. 16A-16B show the results of loss of body weight of the hamsters as a function of days post SARS-CoV-2 infection (see Scheme 4).

FIG. 17 shows the levels of neutralizing antibody titer against the SARS-CoV-2 delta strain in the serum of the hamsters at 35 days (7 days post intranasal challenge with SARS-CoV-2), after intramuscular vaccination of LPMP/SARS-CoV-2 vaccines in the hamsters. Two types of vaccine formulations were administered at D0, at different dosages: LPMP/SARS-CoV-2 A (recLemon LPMP, containing S mRNA 10 μg, 20 μg) and LPMP/SARS-CoV-2 B (recAlgae LPMP, containing S mRNA 10 μg, 20 μg). Control was mock-vaccinated hamsters with EPO mRNA administered at D0.

FIG. 18A-C show the levels of neutralizing antibody titer against the SARS-CoV-2 original, delta, and omicron strains, respectively, in the blood of mice on D49, four weeks after two intramuscular vaccinations of LPMP/SARS-CoV-2 vaccine (recLemon LPMP, containing S mRNA 10 μg) in the mice on D0 and D21. Control was mock-vaccinated mice administered a dose of buffer.

FIG. 19A-H show systemic levels of cytokines and chemokines IFN-gamma, IL-12, CXCL10, TNF-alpha, IL-4, IL-5, IL-13, and IL-10, respectively, in mice at 2, 6, 24, and 48 hours, after a single dose intramuscular vaccination of LPMP/SARS-CoV-2 vaccines (recLemon LPMP, containing S mRNA 10 μg) in the mice. Control was unvaccinated mice administered a dose of buffer at D0.

### DETAILED DESCRIPTION

Featured herein are nucleic acid vaccine compositions (e.g., immunizing/immunogenic compositions), that elicit potent neutralizing antibodies against an antigen of an infectious disease, disorder, or condition (e.g., coronavirus antigens such as a SARS-CoV-2 antigen). These nucleic acid vaccine compositions include one or more polynucleotides (e.g., RNA such as messenger RNA (mRNA)) encoding one or more antigenic polypeptides, formulated within a lipid reconstructed plant messenger packs (LPMPs) comprising natural lipids and an ionizable lipid. The antigenic polypeptides are derived from an infectious agent that causes an infectious disease, disorder, or condition. PMPs are lipid assemblies produced wholly or in part from plant extracellular vesicles (EVs), or segments, portions, or extracts thereof. LPMPs are PMPs derived from a lipid structure wherein the lipid structure is disrupted and reassembled or reconstituted in a liquid phase.

The disclosure also includes a method for making a nucleic acid vaccine, comprising reconstituting a film comprising purified PMP lipids in the presence of a an ionizable lipid to produce a LPMP comprising the ionizable lipid, and loading into the LPMPs with one or more polynucleotides encoding one or more antigenic polypeptide-modified plant messenger packs (PMPs).

### Lipid Reconstructed Plant Messenger Packs (LPMPs)

### Plant Messenger Pack (PMPs)

A PMP is a lipid (e.g., lipid bilayer, unilamellar, or multilamellar structure) structure that includes a plant EV, or segment, portion, or extract (e.g., lipid extract) thereof. Plant EVs refer to an enclosed lipid-bilayer structure that naturally occurs in a plant and that is about 5-2000 nm in diameter. Plant EVs can originate from a variety of plant biogenesis pathways. In nature, plant EVs can be found in the intracellular and extracellular compartments of plants, such as the plant apoplast, the compartment located outside the plasma membrane and formed by a continuum of cell walls and the extracellular space. Alternatively, PMPs can be enriched plant EVs found in cell culture media upon secretion from plant cells. Plant EVs can be separated from plants, thereby providing PMPs, by a variety of methods further described herein. Further, the PMPs can optionally include a therapeutic agent, which can be introduced in vivo or in vitro.

PMPs can include plant EVs, or segments, portions, or extracts, thereof. Optionally, PMPs can also include exogenous lipids (e.g., sterols (e.g., cholesterol or sitosterol), ionizable lipids, and/or PEGylated lipids) in addition to lipids derived from plant EVs. In some embodiments, the plant EVs are about 5-1000 nm in diameter. For example, the PMP can include a plant EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the PMP includes a plant EV, or segment, portion, or extract thereof, that has a mean diameter of about 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the plant EV, or segment, portion, or extract thereof, has a mean diameter of about 50-200 nm. In certain instances, the plant EV, or segment, portion, or extract thereof, has a mean diameter of about 50-300 nm. In certain instances, the plant EV, or segment, portion, or extract thereof, has a mean diameter of about 200-500 nm. In certain instances, the plant EV, or segment, portion, or extract thereof, has a mean diameter of about 30-150 nm.

In some instances, the PMP may include a plant EV, or segment, portion, or extract thereof, that has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, or at least 1000 nm. In some instances, the PMP includes a plant EV, or segment, portion, or extract thereof, that has a mean diameter less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the plant EV, or segment, portion, or extract thereof.

In some instances, the PMP may include a plant EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the PMP may include a plant EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³). In some instances, the PMP may include a plant EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the PMP may include a plant EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

In some instances, the PMP can have the same size as the plant EV or segment, extract, or portion thereof. Alternatively, the PMP may have a different size than the initial plant EV from which the PMP is produced. For example, the PMP may have a diameter of about 5-2000 nm in diameter. For example, the PMP can have a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1200 nm, about 1200-1400 nm, about 1400-1600 nm, about 1600-1800 nm, or about 1800-2000 nm. In some instances, the PMP may have a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 1200 nm, at least 1400 nm, at least 1600 nm, at least 1800 nm, or about 2000 nm. A variety of methods (e.g., a dynamic light scattering method) standard in the art can be used to measure the particle diameter of the PMPs. In some instances, the size of the PMP is determined following loading of a therapeutic agent, or following other modifications to the PMPs.

In some instances, the PMP may have a mean surface area of 77 nm² to 1.3×10⁷ nm² (e.g., 77-100 nm², 100-1000 nm², 1000-1×10⁴ nm², 1×10⁴-1×10⁵ nm², 1×10⁵-1×10⁶ nm², or 1×10⁶-1.3×10⁷ nm²). In some instances, the PMP may have a mean volume of 65 nm³ to 4.2×10⁹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-1×10⁹ nm³, or 1×10⁹-4.2×10⁹ nm³). In some instances, the PMP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 1×10⁷ nm²). In some instances, the PMP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 1×10⁹ nm³, at least 2×10⁹ nm³, at least 3×10⁹ nm³, or at least 4×10⁹ nm³).

In some instances, the PMP may include an intact plant EV. Alternatively, the PMP may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a plant EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the plurality of PMPs can include a plurality of intact plant EVs, a plurality of plant EV segments, portions, or extracts, or a mixture of intact and segments of plant EVs. One skilled in the art will appreciate that the ratio of intact to segmented plant EVs will depend on the particular isolation method used. For example, grinding or blending a plant, or part thereof, may produce PMPs that contain a higher percentage of plant EV segments, portions, or extracts than a non-destructive extraction method, such as vacuum-infiltration.

In instances where, the PMP includes a segment, portion, or extract of a plant EV, the EV segment, portion, or extract may have a mean surface area less than that of an intact vesicle, e.g., a mean surface area less than 77 nm², 100 nm², 1000 nm², 1×10⁴ nm², 1×10⁵ nm², 1×10⁶ nm², or 3.2×10⁶ nm²). In some instances, the EV segment, portion, or extract has a surface area of less than 70 nm², 60 nm², 50 nm², 40 nm², 30 nm², 20 nm², or 10 nm²). In some instances, the PMP may include a plant EV, or segment, portion, or extract thereof, that has a mean volume less than that of an intact vesicle, e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

In instances where the PMP includes an extract of a plant EV, e.g., in instances where the PMP includes lipids extracted (e.g., with chloroform) from a plant EV, the PMP may include at least 1%, 2%, 5%, 10%, 20%, 30%, 40%, 50%, 60%, 70%, 80%, 90%, 95%, 99%, or more than 99%, of lipids extracted (e.g., with chloroform) from a plant EV. The PMPs in the plurality may include plant EV segments and/or plant EV-extracted lipids or a mixture thereof.

### Production of PMPs

PMPs may be produced from plant EVs, or a segment, portion or extract (e.g., lipid extract) thereof, that occur naturally in plants, or parts thereof, including plant tissues or plant cells. An exemplary method for producing PMPs includes (a) providing an initial sample from a plant, or a part thereof, wherein the plant or part thereof comprises EVs; and (b) isolating a crude PMP fraction from the initial sample, wherein the crude PMP fraction has a decreased level of at least one contaminant or undesired component from the plant or part thereof relative to the level in the initial sample. The method can further include an additional step (c) comprising purifying the crude PMP fraction, thereby producing a plurality of pure PMPs, wherein the plurality of pure PMPs have a decreased level of at least one contaminant or undesired component from the plant or part thereof relative to the level in the crude EV fraction. Each production step is discussed in further detail, below. Exemplary methods regarding the isolation and purification of PMPs is found, for example, in Rutter and Innes, Plant Physiol. 173(1): 728-741, 2017; Rutter et al, Bio. Protoc. 7(17): e2533, 2017; Regente et al, J of Exp. Biol. 68(20): 5485-5496, 2017; Mu et al, Mol. Nutr. Food Res., 58, 1561-1573, 2014, and Regente et al, FEBS Letters. 583: 3363-3366, 2009, each of which is herein incorporated by reference.

In some instances, a plurality of PMPs may be isolated from a plant by a process which includes the steps of: (a) providing an initial sample from a plant, or a part thereof, wherein the plant or part thereof comprises EVs; (b) isolating a crude PMP fraction from the initial sample, wherein the crude PMP fraction has a decreased level of at least one contaminant or undesired component from the plant or part thereof relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude PMP fraction, thereby producing a plurality of pure PMPs, wherein the plurality of pure PMPs have a decreased level of at least one contaminant or undesired component from the plant or part thereof relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The PMPs can include a plant EV, or segment, portion, or extract thereof, produced from a variety of plants. PMPs may be produced from any genera of plants (vascular or nonvascular), including but not limited to angiosperms (monocotyledonous and dicotyledonous plants), gymnosperms, ferns, selaginellas, horsetails, psilophytes, lycophytes, algae (e.g., unicellular or multicellular, e.g., archaeplastida), or bryophytes. In certain instances, PMPs can be produced using a vascular plant, for example monocotyledons or dicotyledons or gymnosperms. For example, PMPs can be produced using alfalfa, apple, Arabidopsis, banana, barley, a Brassica species (e.g., Arabidopsis thaliana or Brassica napus), canola, castor bean, chicory, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat or vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, or wheat.

PMPs may be produced using a whole plant (e.g., a whole rosettes or seedlings) or alternatively from one or more plant parts (e.g., leaf, seed, root, fruit, vegetable, pollen, phloem sap, or xylem sap). For example, PMPs can be produced using shoot vegetative organs/structures (e.g., leaves, stems, or tubers), roots, flowers and floral organs/structures (e.g., pollen, bracts, sepals, petals, stamens, carpels, anthers, or ovules), seed (including embryo, endosperm, or seed coat), fruit (the mature ovary), sap (e.g., phloem or xylem sap), plant tissue (e.g., vascular tissue, ground tissue, tumor tissue, or the like), and cells (e.g., single cells, protoplasts, embryos, callus tissue, guard cells, egg cells, or the like), or progeny of same. For instance, the isolation step may involve (a) providing a plant, or a part thereof. In some examples, the plant part is an Arabidopsis leaf. The plant may be at any stage of development. For example, the PMPs can be produced using seedlings, e.g., 1 week, 2 week, 3 week, 4 week, 5 week, 6 week, 7 week, or 8 week old seedlings (e.g., Arabidopsis seedlings). Other exemplary PMPs can include PMPs produced using roots (e.g., ginger roots), fruit juice (e.g., grapefruit juice), vegetables (e.g., broccoli), pollen (e.g., olive pollen), phloem sap (e.g., Arabidopsis phloem sap), or xylem sap (e.g., tomato plant xylem sap).

In some embodiments, the PMPs are produced from algae or lemon.

PMPs can be produced using a plant, or part thereof, by a variety of methods. Any method that allows release of the EV-containing apoplastic fraction of a plant, or an otherwise extracellular fraction that contains PMPs comprising secreted EVs (e.g., cell culture media) is suitable in the present methods. EVs can be separated from the plant or plant part by either destructive (e.g., grinding or blending of a plant, or any plant part) or non-destructive (washing or vacuum infiltration of a plant or any plant part) methods. For instance, the plant, or part thereof, can be vacuum-infiltrated, ground, blended, or a combination thereof to isolate EVs from the plant or plant part, thereby producing PMPs. For instance, the isolating step may involve vacuum infiltrating the plant (e.g., with a vesicle isolation buffer) to release and collect the apoplastic fraction. Alternatively, the isolating step may involve grinding or blending the plant to release the EVs, thereby producing PMPs.

Upon isolating the plant EVs, thereby producing PMPs, the PMPs can be separated or collected into a crude PMP fraction (e.g., an apoplastic fraction). For instance, the separating step may involve separating the plurality of PMPs into a crude PMP fraction using centrifugation (e.g., differential centrifugation or ultracentrifugation) and/or filtration to separate the plant PMP-containing fraction from large contaminants, including plant tissue debris or plant cells. As such, the crude PMP fraction will have a decreased number of large contaminants, including plant tissue debris or plant cells, as compared to the initial sample from the plant or plant part. Depending on the method used, the crude PMP fraction may additionally comprise a decreased level of plant cell organelles (e.g., nuclei, mitochondria or chloroplasts), as compared to the initial sample from the plant or plant part.

In some instances, the isolating step may involve separating the plurality of PMPs into a crude PMP fraction using centrifugation (e.g., differential centrifugation or ultracentrifugation) and/or filtration to separate the PMP-containing fraction from plant cells or cellular debris. In such instances, the crude PMP fraction will have a decreased number of plant cells or cellular debris, as compared to the initial sample from the source plant or plant part.

The crude PMP fraction can be further purified by additional purification methods to produce a plurality of pure PMPs. For example, the crude PMP fraction can be separated from other plant components by ultracentrifugation, e.g., using a density gradient (iodixanol or sucrose) and/or use of other approaches to remove aggregated components (e.g., precipitation or size-exclusion chromatography). The resulting pure PMPs may have a decreased level of contaminants or other undesired components from the source plant (e.g., one or more non-PMP components, such as protein aggregates, nucleic acid aggregates, protein-nucleic acid aggregates, free lipoproteins, lipido-proteic structures), nuclei, cell wall components, cell organelles, or a combination thereof) relative to one or more fractions generated during the earlier separation steps, or relative to a pre-established threshold level, e.g., a commercial release specification. For example, the pure PMPs may have a decreased level (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) of plant organelles or cell wall components relative to the level in the initial sample. In some instances, the pure PMPs are substantially free (e.g., have undetectable levels) of one or more non-PMP components, such as protein aggregates, nucleic acid aggregates, protein-nucleic acid aggregates, free lipoproteins, lipido-proteic structures), nuclei, cell wall components, cell organelles, or a combination thereof. The PMPs may be at a concentration of, e.g., 1×10⁹, 5×10⁹, 1×10¹⁰, 5×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, or more than 1×10¹³ PMPs/mL.

For example, protein aggregates may be removed from PMPs. For example, the PMPs can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the PMPs can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example, NaCl can be added to the PMPs until it is at, e.g., 1 mol/L. The solution can then be filtered to isolate the PMPs. Alternatively, aggregates are solubilized by increasing the temperature. For example, the PMPs can be heated under mixing until the solution has reached a uniform temperature of, e.g., 50° C. for 5 minutes. The PMP mixture can then be filtered to isolate the PMPs. Alternatively, soluble contaminants from PMP solutions can be separated by size-exclusion chromatography column according to standard procedures, where PMPs elute in the first fractions, whereas proteins and ribonucleoproteins and some lipoproteins are eluted later. The efficiency of protein aggregate removal can be determined by measuring and comparing the protein concentration before and after removal of protein aggregates via BCA/Bradford protein quantification.

Any of the production methods described herein can be supplemented with any quantitative or qualitative methods known in the art to characterize or identify the PMPs at any step of the production process. PMPs may be characterized by a variety of analysis methods to estimate PMP yield, PMP concentration, PMP purity, PMP composition, or PMP sizes. PMPs can be evaluated by a number of methods known in the art that enable visualization, quantitation, or qualitative characterization (e.g., identification of the composition) of the PMPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify plant EV markers present on the PMP, such as markers disclosed in the Appendix. To aid in analysis and characterization, of the PMP fraction, the PMPs can additionally be labelled or stained. For example, the PMPs can be stained with 3,3′-dihexyloxacarbocyanine iodide (DIOC₆), a fluorescent lipophilic dye, PKH67 (Sigma Aldrich); Alexa Fluor®488 (Thermo Fisher Scientific), or DyLight™ 800 (Thermo Fisher). In the absence of sophisticated forms of nanoparticle tracking, this relatively simple approach quantifies the total membrane content and can be used to indirectly measure the concentration of PMPs (Rutter and Innes, Plant Physiol. 173(1): 728-741, 2017; Rutter et al, Bio. Protoc. 7(17): e2533, 2017). For more precise measurements, and to assess the size distributions of PMPs, nanoparticle tracking can be used.

During the production process, the PMPs can optionally be prepared such that the PMPs are at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the EV level in a control or initial sample. The PMPs may make up about 0.1% to about 100% of the PMP composition, such as any one of about 0.01% to about 100%, about 1% to about 99.9%, about 0.1% to about 10%, about 1% to about 25%, about 10% to about 50%, about 50% to about 99%, or about 75% to about 100%. In some instances, the composition includes at least any of 0.1%, 0.5%, 1%, 5%, 10%, 20%, 30%, 40%, 50%, 60%, 70%, 80%, 90%, 95%, or more PMPs, e.g., as measured by wt/vol, percent PMP protein composition, and/or percent lipid composition (e.g., by measuring fluorescently labelled lipids). In some instances, the concentrated agents are used as commercial products, e.g., the final user may use diluted agents, which have a substantially lower concentration of active ingredient. In some embodiments, the composition is formulated as an agricultural concentrate formulation, e.g., an ultra-low-volume concentrate formulation.

### Lipid Reconstructed Plant Messenger Packs (LPMPs)

A lipid reconstructed PMP (LPMP) is used herein. LPMP refers to a PMP that has been derived from a lipid structure (e.g., a lipid bilayer, unilamellar, multilamellar structure; e.g., a vesicular lipid structure) derived from (e.g., enriched, isolated or purified from) a plant source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LPMP, as is described herein. The method may, if desired, further comprise sonication, freeze/thaw treatment, and/or lipid extrusion, e.g., to reduce the size of the reconstituted PMPs. Alternatively, LPMPs may be produced using a microfluidic device (such as a NanoAssemblr® IGNITE™ microfluidic instrument (Precision NanoSystems)).

In some embodiments, the LPMPs are produced by a process which comprises the steps of (a) providing a plurality of purified PMPs (e.g., PMPs purified as described in Section IA herein); (b) processing the plurality of PMPs to produce a lipid film; (c) reconstituting the lipid film in an organic solvent or solvent combination, thereby producing a lipid solution; and (d) processing the lipid solution of step (c) in a microfluidics device comprising an aqueous phase, thereby producing the LPMPs.

In some instances, processing the plurality of PMPs to produce a lipid film includes extracting lipids from the plurality of PMPs, e.g., extracting lipids using the Bligh-Dyer method (Bligh and Dyer, J Bio/chem Physiol, 37: 911-917, 1959). The extracted lipids may be provided as a stock solution, e.g., a solution in chloroform:methanol. Producing the lipid film may comprise, e.g., evaporation of the solvent with a stream of inert gas (e.g., nitrogen).

### Natural Lipids

A LPMP may comprise between 10% and 100% lipids derived from the lipid structure from the plant source (e.g., lemon or algae), e.g., may contain at least 10%, at least 20%, at least 30%, at least 40%, at least 50%, at least 60%, at least 70%, at least 80%, at least 90%, at least 95%, or 100% lipids derived from the lipid structure from the plant source. A LPMP may comprise all or a fraction of the lipid species present in the lipid structure from the plant source (e.g., lemon or algae), e.g., it may contain at least 10%, at least 20%, at least 30%, at least 40%, at least 50%, at least 60%, at least 70%, at least 80%, at least 90%, or 100% of the lipid species present in the lipid structure from the plant source. A LPMP may comprise none, a fraction, or all of the protein species present in the lipid structure from the plant source (e.g., lemon or algae), e.g., may contain 0%, less than 1%, less than 5%, less than 10%, less than 15%, less than 20%, less than 30%, less than 40%, less than 50%, less than 60%, less than 70%, less than 80%, less than 90%, less than 100%, or 100% of the protein species present in the lipid structure from the plant source (e.g., lemon or algae). In some instances, the lipid bilayer of the LPMP does not contain proteins. In some instances, the lipid structure of the LPMP contains a reduced amount of proteins relative to the lipid structure from the plant source.

In some embodiments, the natural lipids of the LPMPs are extracted from lemon or algae.

### Exogenous Lipids

The LPMPs may be modified to contain a heterologous agent (e.g., a cell-penetrating agent) that is capable of increasing cell uptake (e.g., animal cell uptake (e.g., mammalian cell uptake, e.g., human cell uptake), plant cell uptake, bacterial cell uptake, or fungal cell uptake) relative to an unmodified LPMP. For example, the modified LPMPs may include (e.g., be loaded with, e.g., encapsulate or be conjugated to) or be formulated with (e.g., be suspended or resuspended in a solution comprising) a plant cell-penetrating agent, such as an ionizable lipid. Each of the modified LPMPs may comprise at least 1%, 5%, 10%, 20%, 30%, 40%, 50%, 60%, 70%, 80%, 90%, or more than 90% ionizable lipid.

LPMPs may include one or more exogenous lipids, e.g., lipids that are exogenous to the plant (e.g., originating from a source that is not the plant or plant part from which the LPMP is produced). The lipid composition of the LPMP may include 0%, less than 1%, or at least 1%, 2%, 5%, 10%, 15%, 20%, 25%, 30%, 40%, 50%, 60%, 70%, 80%, 90%, 95%, or more than 95% exogenous lipid. In some examples, the exogenous lipid (e.g., ionizable lipid) is added to amount to 25% or 40% (w/w) of total lipids in the preparation. In some examples, the exogenous lipid is added to the preparation prior to step (b), e.g., mixed with extracted PMP lipids prior to step (b).

Exemplary exogenous lipids include ionizable lipids.

Exogenous lipids may also include cationic lipids.

In some instances, the exogenous lipid may be an ionizable lipid or cationic lipid chosen from 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), DLin-MC3-DMA (MC3), dioleoyl-3-trimethylammonium propane (DODAP), DC-cholesterol, DOTAP, Ethyl PC, GL67, DLin-KC2-DMA (KC2), MD1 (cKK-E12), OF2, EPC, ZA3-Ep10, TT3, LP01, 5A2-SC8, Lipid 5 (Moderna), a cationic sulfonamide amino lipid, an amphiphilic zwitterionic amino lipid, DODAC, DOBAQ, YSK05, DOBAT, DOBAQ, DOPAT, DOMPAQ, DOAAQ, DMAP-BLP, DLinDMA, DODMA, DOTMA, DSDMA, DOSPA, DODAC, DOBAQ, DMRIE, DOTAP-cholesterol, GL67A, and 98N12-5 or a combination thereof.

In some embodiments, the exogenous lipid may be an ionizable lipid or cationic lipid chosen from C12-200, MC3, DODAP, DC-cholesterol, DOTAP, Ethyl PC, GL67, KC2, MD1, OF2, EPC, ZA3-Ep10, TT3, LP01, 5A2-SC8, Lipid 5 (Moderna), a cationic sulfonamide amino lipid, and an amphiphilic zwitterionic amino lipid or a combination thereof. In some embodiments, the ionizable lipid is chosen from C12-200, MC3, DODAP, and DC-cholesterol or combinations thereof. In some instances, the ionizable lipid is an ionizable lipid. In some embodiments, the ionizable lipid is 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200) or (6Z,9Z,28Z,31Z)-Heptatriaconta-6,9,28,31-tetraen-19-yl 4-(dimethylamino)butanoate, DLin-MC3-DMA (MC3). In some instances, the exogenous lipid is a cationic lipid. In some embodiments, the cationic lipid is DC-cholesterol or dioleoyl-3-trimethylammonium propane (DOTAP).

In some instances, the LPMPs comprise at least 1%, 5%, 10%, 20%, 30%, 40%, 50%, 60%, 70%, 80%, 90%, or more than 90% ionizable lipid.

In some instances, the LPMPs comprise a molar ratio of least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 30%-75% ionizable lipid (e.g., about 30%-75% ionizable lipid). In some embodiments, the LPMP comprises 25% C12-200. In some embodiments, the LPMP comprises a molar ratio of 35% C12-200. In some embodiments, the LPMP comprises a molar ratio of 50% C12-200. In some embodiments, the LPMP comprises 40% MC3. In some embodiments, the LPMP comprises a molar ratio of 50% C12-200. In some embodiments, the LPMP comprises 20% or 40% DC-cholesterol.

In some embodiments, the LPMP comprises 25% or 40% DOTAP.

The agent may increase uptake of the LPMP as a whole or may increase uptake of a portion or component of the LPMP (e.g., the nucleic acid vaccine) carried by the LPMP. The degree to which cell uptake is increased may vary depending on the plant or plant part to which the composition is delivered, the LPMP formulation, and other modifications made to the LPMP, For example, the modified LPMPs may have an increased cell uptake (e.g., animal cell uptake, plant cell uptake, bacterial cell uptake, or fungal cell uptake) of at least 1%, 2%, 5%, 10%, 15%, 20%, 30%, 40%, 50%, 60%, 70%, 80%, 90%, or 100% relative to an unmodified LPMP. In some instances, the increased cell uptake is an increased cell uptake of at least 2×-fold, 4×-fold, 5×-fold, 10×-fold, 100×-fold, or 1000×-fold relative to an unmodified LPMP.

In some embodiments, a LPMP that has been modified with a ionizable lipid more efficiently encapsulates a negatively charged a polynucleotide than a LPMP that has not been modified with an ionizable lipid. In some aspects, a LPMP that has been modified with an ionizable lipid has altered biodistribution relative to a LPMP that has not been modified with an ionizable lipid. In some aspects, a LPMP that has been modified with an ionizable lipid has altered (e.g., increased) fusion with an endosomal membrane of a target cell relative to a LPMP that has not been modified with an ionizable lipid.

### Ionizable Lipids

In some embodiments, the ionizable lipid has at least one (e.g., one, two, three, four or all five) of the characteristics listed below:
(i) at least 2 ionizable amines (e.g., at least 2, at least 3, at least 4, at least 5, at least 6, or more than 6 ionizable amines, e.g., 2, 3, 4, 5, 6, 7, 8, 9, 10, 11, 12, or more than 12 ionizable amines);
(ii) at least 3 lipid tails (e.g., at least 3, at least 4, at least 5, at least 6, or more than 6 lipid tails, e.g., 2, 3, 4, 5, 6, 7, 8, 9, 10, 11, 12, or more than 12 lipid tails), wherein each of the lipid tails is independently at least 6 carbon atoms in length (e.g., at least 6, at least 7, at least 8, at least 9, at least 10, at least 11, at least 12, at least 13, at least 14, at least 15, at least 16, at least 17, at least 18, or more than 18 carbon atoms in length, e.g., 6, 7, 8, 9, 10, 11, 12, 13, 14, 15, 16, 17, 18, 19, 20, 21, 22, 23, 24, 25, or more than 25 carbon atoms in length);
(iii) an acid dissociation constant (pKa) of from about 4.5 to about 7.5 (e.g., a pKa of about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 (e.g., a pKa of from about 6.5 and about 7.5 (e.g., a pKa of about 6.5, 6.6, 6.7, 6.8, 6.9, 7.0, 7.1, 7.2, 7.3, 7.4, or 7.5));
(iv) an ionizable amine and a heteroorganic group; and
(v) an N:P (amines of ionizable lipid:phosphates of mRNA) ratio of at least 10;

In some embodiments, the ionizable lipid is not selected from 1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5 (Moderna), and 98N12-5.

In some embodiments, the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

In some embodiments, the ionizable lipid is an ionizable amine and a heteroorganic group. In some embodiments, the heteroorganic group is hydroxyl. In some embodiments, the heteroorganic group comprises a hydrogen bond donor. In some embodiments, the heteroorganic group comprises a hydrogen bond acceptor. In some embodiments, the heteroorganic group is —OH, —SH, —(CO)H, —CO₂H, —NH₂, —CONH₂, optionally substituted C₁-C₆ alkoxy, or fluorine.

In some embodiments, the ionizable lipid is an ionizable amine and a heteroorganic group separated by a chain of at least two atoms

In some embodiments, the ionizable lipid is represented by the following formula I:

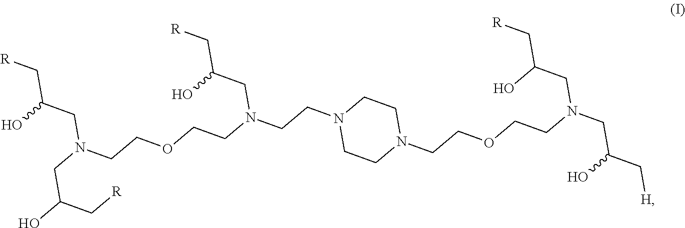

wherein R is C8-C14 alkyl group.

In some embodiments, a lipid membrane of the LPMPs comprises at least 35% of the lipid of formula I, e.g., at least 40%, 45%, 50%, 55%, 60%, 65%, 70%, 75% 80%, 85%, 90%, or more than 90% of the lipid of formula I, e.g., 35%-40%, 40%-50%, 50%-60%, 60%-70%, 70%-80%, or 80%-90% of the lipid of formula I.

In some instances, the LPMPs comprise at least 1%, 5%, 10%, 20%, 30%, 40%, 50%, 60%, 70%, 80%, 90%, or more than 90% ionizable lipid.

In some instances, the LPMPs comprise a molar ratio of at least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 25%-75% ionizable lipid (e.g., about 25%-75% ionizable lipid).

The ionizable lipid described herein may include an amine core described herein substituted with one or more (e.g., 1, 2, 3, 4, 5, or 6) lipid tails. In some embodiments, the ionizable lipid described herein include at least 3 lipid tails. A lipid tail may be a C₈-C₁₈ hydrocarbon (e.g., C₆-C₁₈ alkyl or C₆-C₁₈ alkanoyl). An amine core may be substituted with one or more lipid tails at a nitrogen atom (e.g., one hydrogen atom attached to the nitrogen atom may be replaced with a lipid tail).

In some embodiments, the amine core has a structure of:

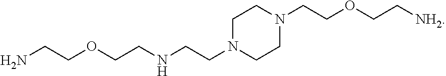 [SMILES: NCCOCCNCCN1CCN(CCOCCN)CC1]

In some embodiments, the amine core has a structure of:

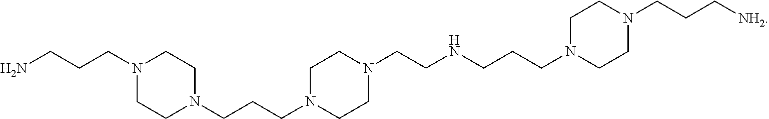 [SMILES: NCCCN1CCN(CCCNCCN2CCN(CCCN3CCN(CCCN)CC3)CC2)CC1]

In some embodiments, the amine core has a structure of:

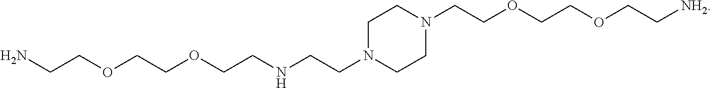 [SMILES: NCCOCCOCCNCCN1CCN(CCOCCOCCN)CC1]

In some embodiments, the amine core has a structure of:

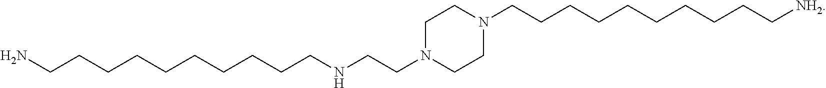 [SMILES: NCCCCCCCCCCNCCN1CCN(CCCCCCCCCCN)CC1]

In some embodiments, the amine core has a structure of:

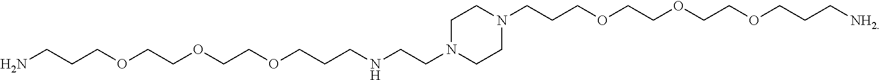 [SMILES: NCCCOCCOCCOCCCNCCN1CCN(CCCOCCOCCOCCCN)CC1]

In some embodiments, the amine core has a structure of:

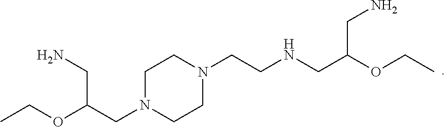 [SMILES: CCOC(CN)CNCCN1CCN(CC(CN)OCC)CC1]

In some embodiments, the amine core has a structure of:

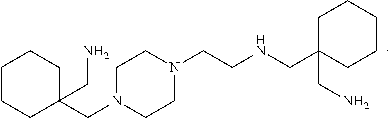 [SMILES: NCC1(CNCCN2CCN(CC3(CN)CCCCC3)CC2)CCCCC1]

In some embodiments, the amine core has a structure of:

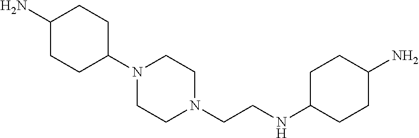 [SMILES: NC1CCC(NCCN2CCN(C3CCC(N)CC3)CC2)CC1]

Other suitable lipids for use in the nucleic acid vaccine and methods for making and using thereof include the lipids as described in International Patent Publication WO 2016/118725, which is incorporated herein by reference in its entirety.

In certain embodiments, the nucleic acid vaccine and methods for making and using thereof include a lipid having a compound structure of:

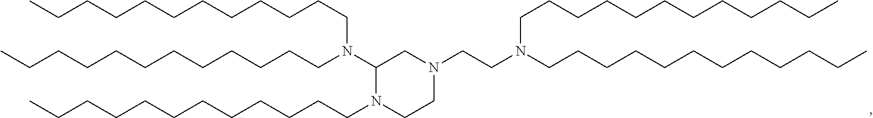 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable lipids for use in the nucleic acid vaccine and methods for making and using thereof include the lipids as described in International Patent Publication WO 2016/118724, which is incorporated herein by reference in its entirety.

In certain embodiments, the nucleic acid vaccine and methods for making and using thereof include a lipid having a compound structure of:

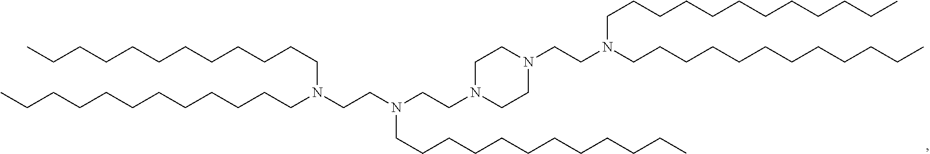 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable lipids for use in the nucleic acid vaccine and methods for making and using thereof include a lipid having the formula of 14,25-ditridecyl 15,18,21,24-tetraaza-octatriacontane, and pharmaceutically acceptable salts thereof.

Other suitable lipids for use in the nucleic acid vaccine and methods for making and using thereof include the lipids as described in International Patent Publications WO 2013/063468 and WO 2016/205691, each of which is incorporated herein by reference in its entirety.

In some embodiments, the nucleic acid vaccine and methods for making and using thereof include a lipid of the following formula:

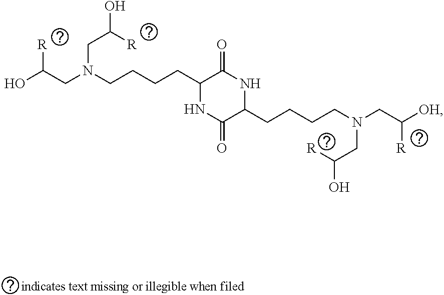

or pharmaceutically acceptable salts thereof, wherein each instance of R^{L} is independently optionally substituted C6-C40 alkenyl.

In certain embodiments, the nucleic acid vaccine and methods for making and using thereof include a lipid having a compound structure of:

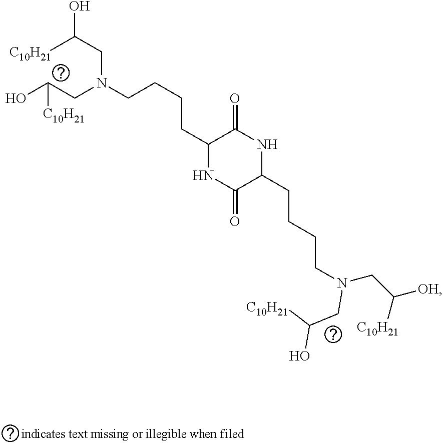

pharmaceutically acceptable salts thereof.

In certain embodiments, the nucleic acid vaccine and methods for making and using thereof include a lipid having a compound structure of:

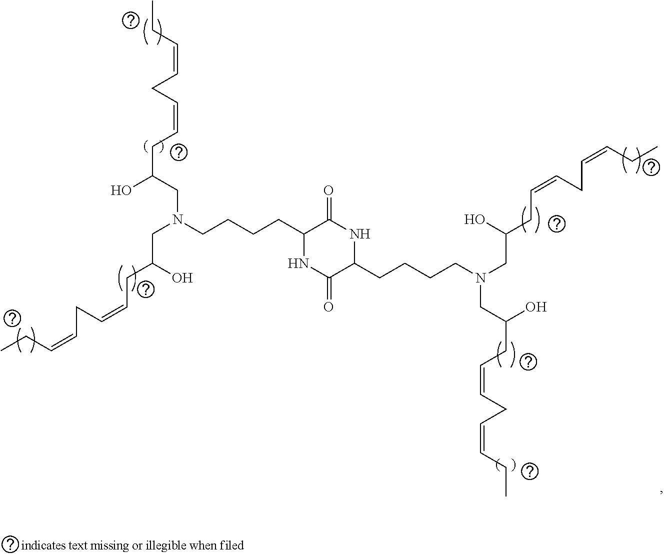

and pharmaceutically acceptable salts thereof.

In certain embodiments, the nucleic acid vaccine and methods for making and using thereof include a lipid having a compound structure of:

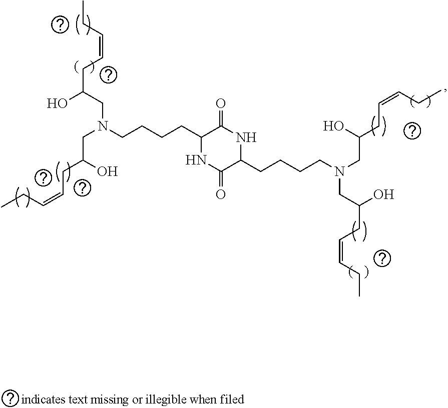

and pharmaceutically acceptable salts thereof.

In certain embodiments, the nucleic acid vaccine and methods for making and using thereof include a lipid having a compound structure of:

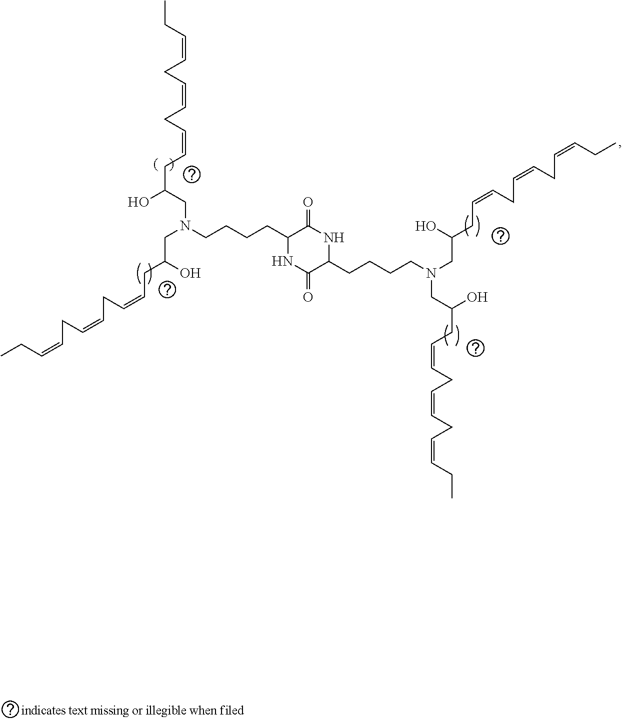

and pharmaceutically acceptable salts thereof.

Other suitable lipids for use in the nucleic acid vaccine and methods for making and using thereof include the lipids as described in International Patent Publication WO 2015/184256, which is incorporated herein by reference in its entirety. In some embodiments, the nucleic acid vaccine and methods for making and using thereof include a lipid of the following formula:

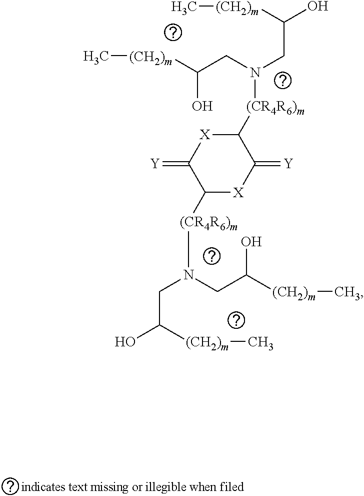

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen.

In certain embodiments, the nucleic acid vaccine and methods for making and using thereof include a lipid, “Target 23”, having a compound structure of:

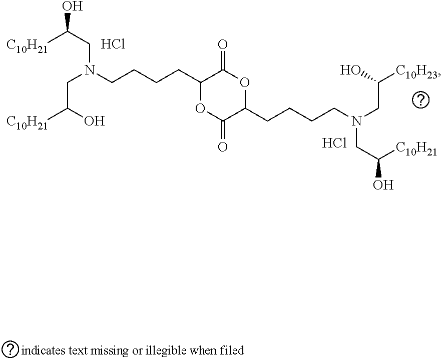

(Target 23) and pharmaceutically acceptable salts thereof.

Other suitable lipids for use in the nucleic acid vaccine and methods for making and using thereof include the lipids as described in International Patent Publication WO 2016/004202, which is incorporated herein by reference in its entirety.

In some embodiments, the nucleic acid vaccine and methods for making and using thereof include a lipid having the compound structure:

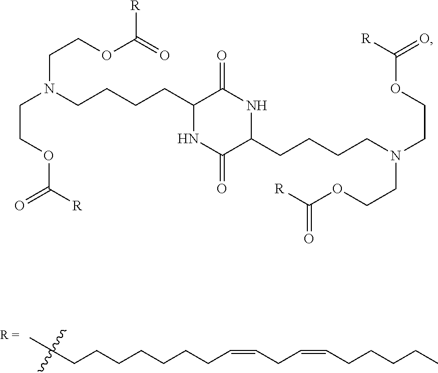

or a pharmaceutically acceptable salt thereof.

In some embodiments, the nucleic acid vaccine and methods for making and using thereof include a lipid having the compound structure:

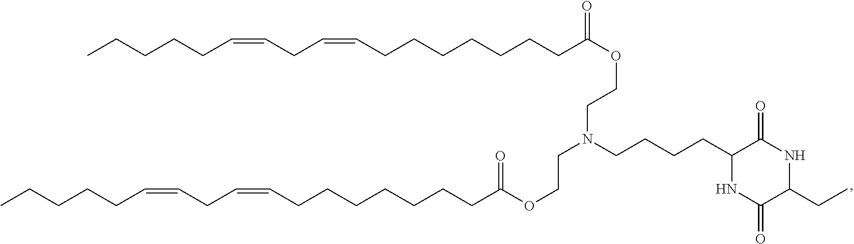 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof.

In some embodiments, the nucleic acid vaccine and methods for making and using thereof include a lipid having the compound structure:

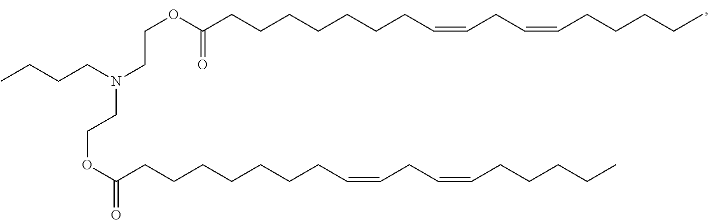 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable lipids for use in the nucleic acid vaccine and methods for making and using thereof include lipids as described in U.S. Provisional Patent Application Ser. No. 62/758,179, which is incorporated herein by reference in its entirety.

In some embodiments, the nucleic acid vaccine and methods for making and using thereof include a lipid of the following formula:

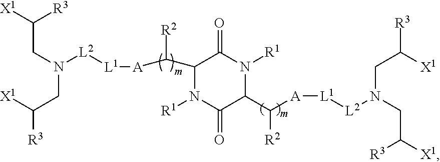

or a pharmaceutically acceptable salt thereof, wherein each R¹ and R² is independently H or C1-C6 aliphatic; each m is independently an integer having a value of 1 to 4; each A is independently a covalent bond or arylene; each L¹ is independently an ester, thioester, disulfide, or anhydride group; each L² is independently C2-C10 aliphatic; each X¹ is independently H or OH; and each R³ is independently C6-C20 aliphatic.

In some embodiments, the nucleic acid vaccine and methods for making and using thereof include a lipid of the following formula:

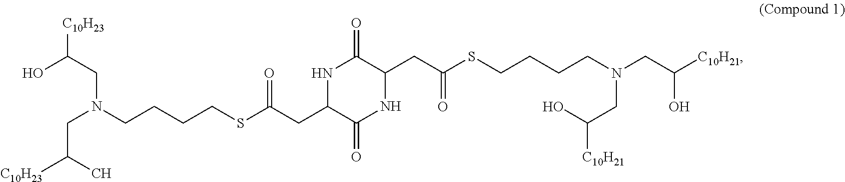

or a pharmaceutically acceptable salt thereof.

In some embodiments, the nucleic acid vaccine and methods for making and using thereof include a lipid of the following formula:

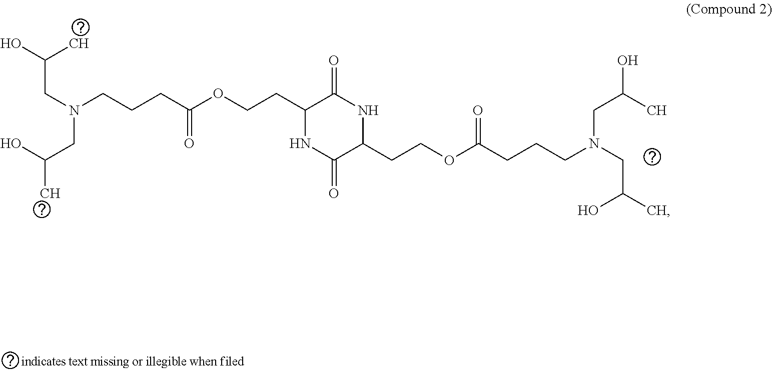

or a pharmaceutically acceptable salt thereof.

In some embodiments, the nucleic acid vaccine and methods for making and using thereof include a lipid of the following formula:

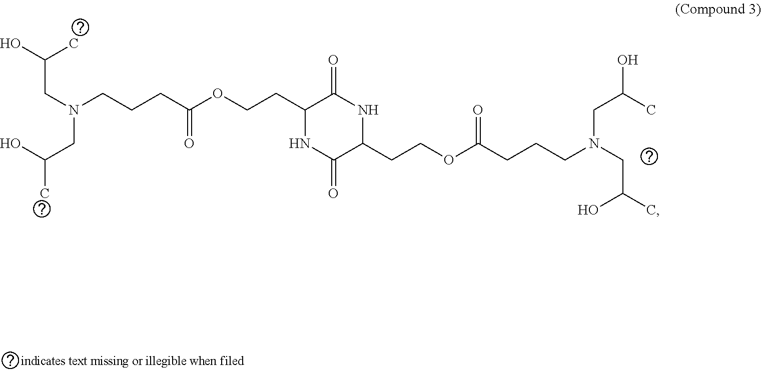

or a pharmaceutically acceptable salt thereof.

Other suitable lipids for use in the nucleic acid vaccine and methods for making and using thereof include the lipids as described in J. McClellan, M. C. King, Cell 2010, 141, 210-217 and in Whitehead et al., Nature Communications (2014) 5:4277, which is incorporated herein by reference in its entirety.

In certain embodiments, the lipids of the nucleic acid vaccine and methods for making and using thereof include a lipid having a compound structure of:

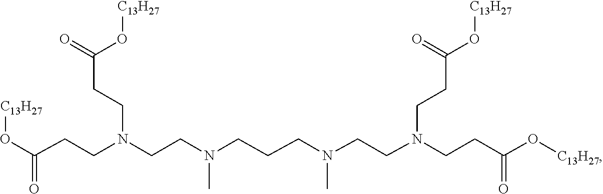

and pharmaceutically acceptable salts thereof.

Other suitable lipids for use in the nucleic acid vaccine and methods for making and using thereof include the lipids as described in International Patent Publication WO 2015/199952, which is incorporated herein by reference in its entirety.

In some embodiments, the nucleic acid vaccine and methods for making and using thereof include a lipid having the compound structure:

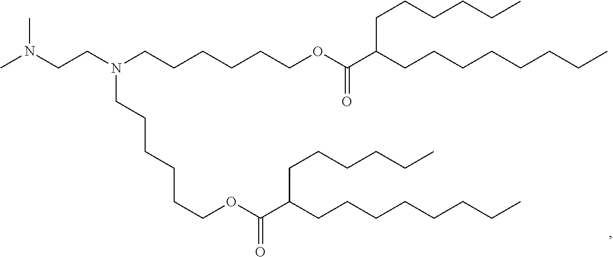 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof.

In some embodiments, the nucleic acid vaccine and methods for making and using thereof include a lipid having the compound structure:

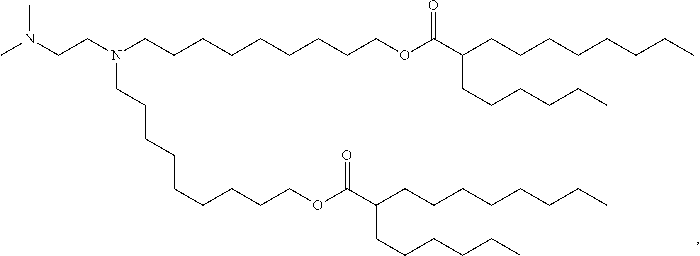 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof.

In some embodiments, the nucleic acid vaccine and methods for making and using thereof include a lipid having the compound structure:

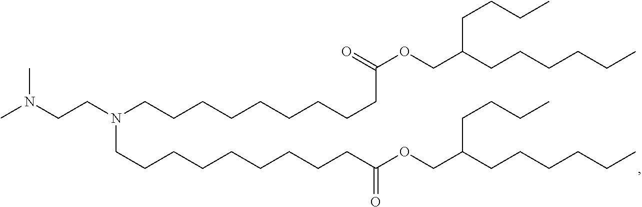 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof.

In some embodiments, the nucleic acid vaccine and methods for making and using thereof include a lipid having the compound structure;

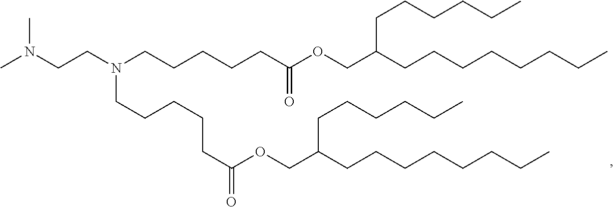 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof.

In some embodiments, the nucleic acid vaccine and methods for making and using thereof include a lipid having the compound structure:

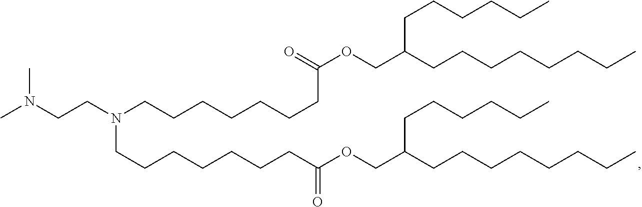 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof.

In some embodiments, the nucleic acid vaccine and methods for making and using thereof include a lipid having the compound structure:

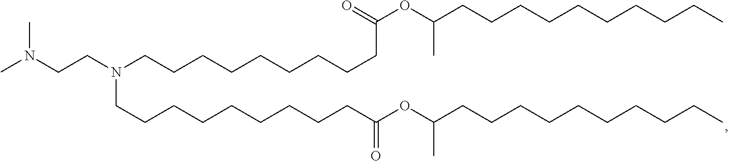 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof.

In some embodiments, the nucleic acid vaccine and methods for making and using thereof include a lipid having the compound structure:

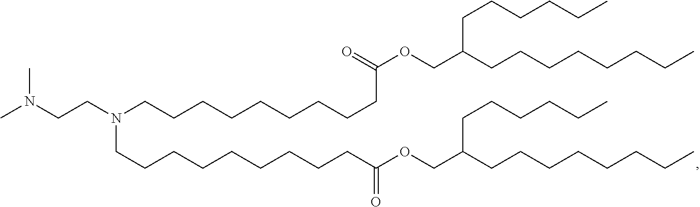 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof.

In some embodiments, the nucleic acid vaccine and methods for making and using thereof include a lipid having the compound structure:

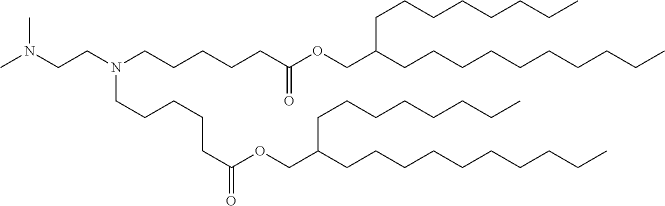 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof.

In some embodiments, the nucleic acid vaccine and methods for making and using thereof include a lipid having the compound structure:

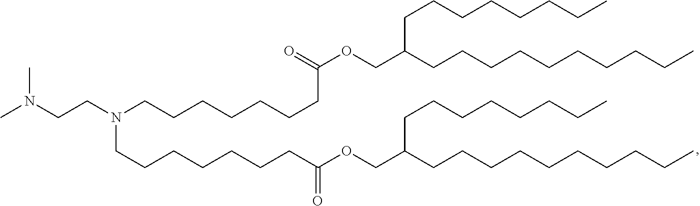 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof.

In some embodiments, the nucleic acid vaccine and methods for making and using thereof include a lipid having the compound structure:

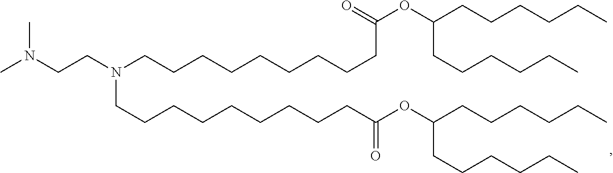 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof.

In some embodiments, the nucleic acid vaccine and methods for making and using thereof include a lipid having the compound structure:

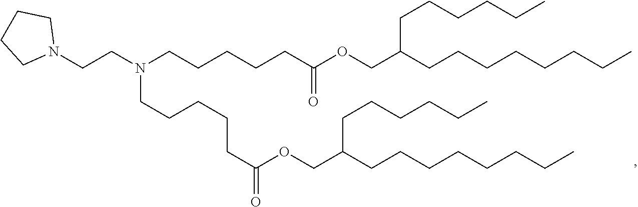 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof.

In some embodiments, the nucleic acid vaccine and methods for making and using thereof include a lipid having the compound structure:

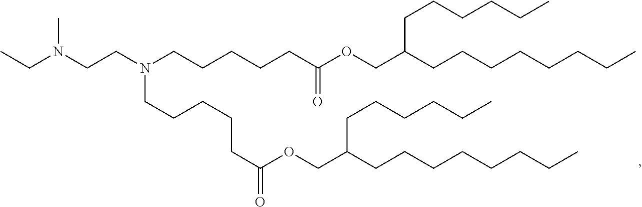 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof.

In some embodiments, the nucleic acid vaccine and methods for making and using thereof include a lipid having the compound structure:

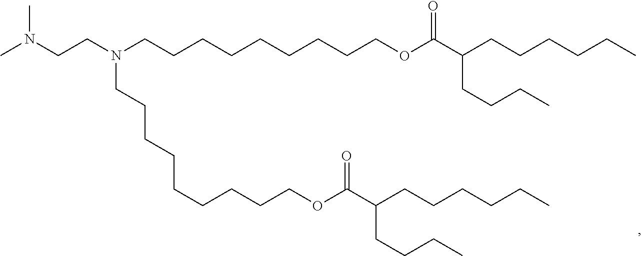 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable lipids for use in the nucleic acid vaccine and methods for making and using thereof include the lipids as described in International Patent Publication WO 2017/004143, which is incorporated herein by reference in its entirety.

In some embodiments, the nucleic acid vaccine and methods for making and using thereof include a lipid having the compound structure:

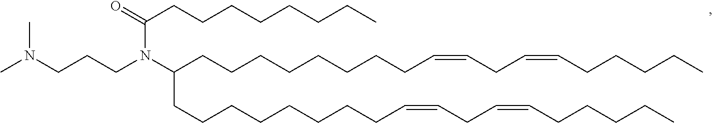 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof.

In some embodiments, the nucleic acid vaccine and methods for making and using thereof include a lipid having the compound structure:

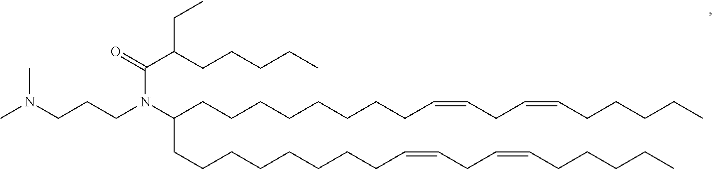 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof.

In some embodiments, the nucleic acid vaccine and methods for making and using thereof include a lipid having the compound structure:

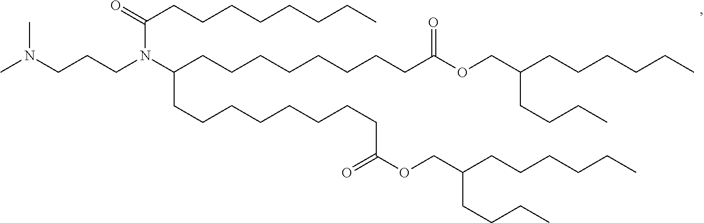 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof.

In some embodiments, the nucleic acid vaccine and methods for making and using thereof include a lipid having the compound structure:

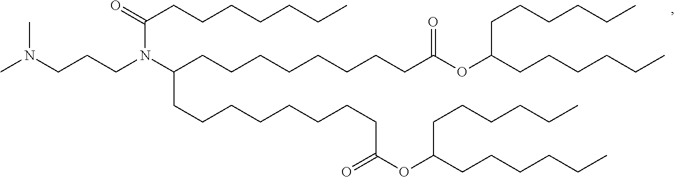 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the nucleic acid vaccine and methods for making and using thereof include a lipid having the compound structure:

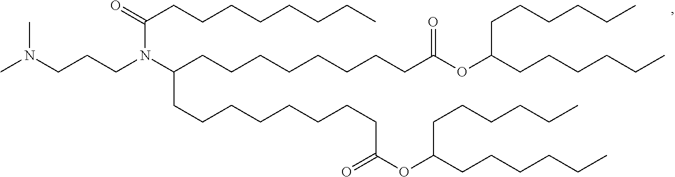 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the nucleic acid vaccine and methods for making and using thereof include a lipid having the compound structure:

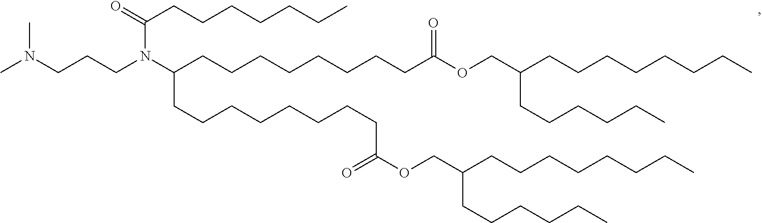 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof.

In some embodiments, the nucleic acid vaccine and methods for making and using thereof include a lipid having the compound structure:

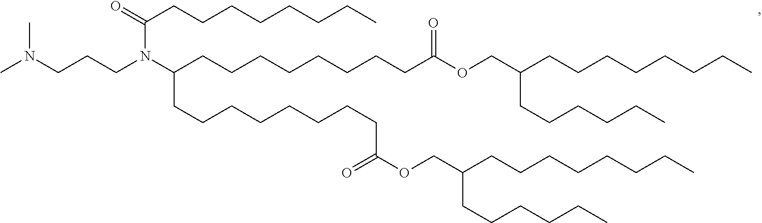 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof.

In some embodiments, the nucleic acid vaccine and methods for making and using thereof include a lipid having the compound structure:

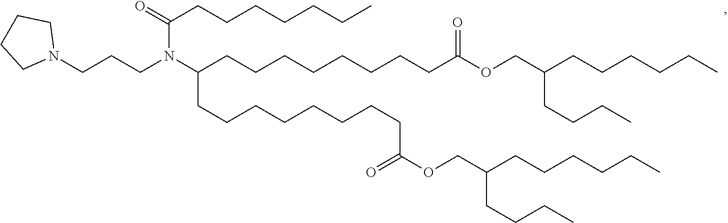 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the nucleic acid vaccine and methods for making and using thereof include a lipid having the compound structure:

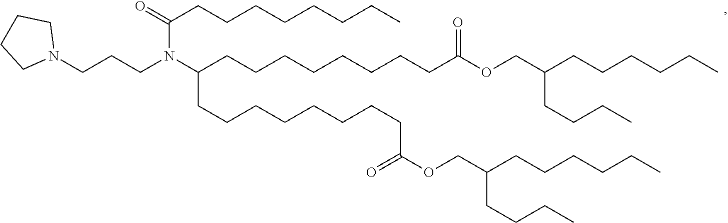 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the nucleic acid vaccine and methods for making and using thereof include a lipid having the compound structure:

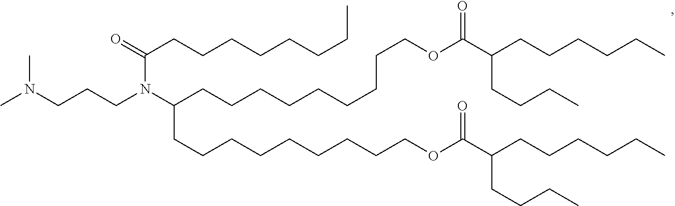 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof.

In some embodiments, the nucleic acid vaccine and methods for making and using thereof include a lipid having the compound structure:

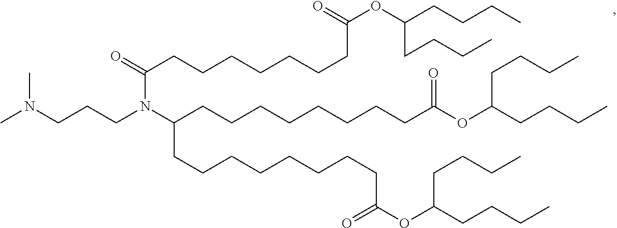 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof.

In some embodiments, the nucleic acid vaccine and methods for making and using thereof include a lipid having the compound structure:

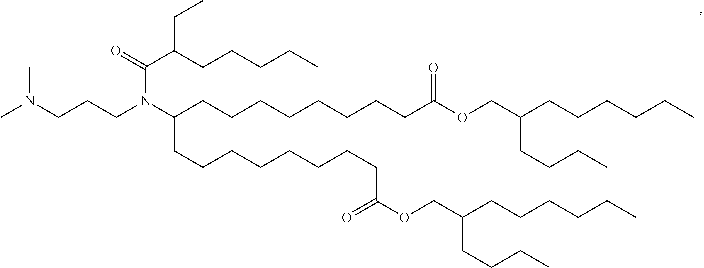 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof.

In some embodiments, the nucleic acid vaccine and methods for making and using thereof include a lipid having the compound structure:

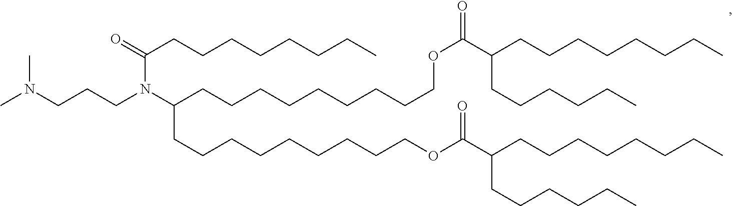 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof.

In some embodiments, the nucleic acid vaccine and methods for making and using thereof include a lipid having the compound structure:

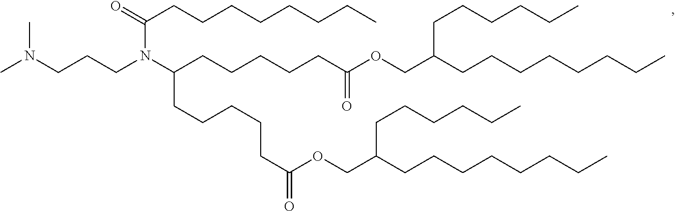 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof.

In some embodiments, the nucleic acid vaccine and methods for making and using thereof include a lipid having the compound structure:

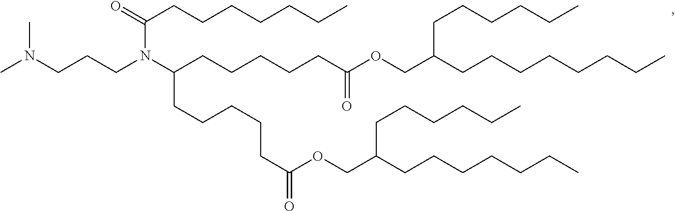 [SMILES: CCCCCCCCC(CCCCCC)COC(=O)CCCCCC(CCCCCC(=O)OCC(CCCCCC)CCCCCCC)N(CCCN(C)C)C(=O)CCCCCCC]

and pharmaceutically acceptable salts thereof.

In some embodiments, the nucleic acid vaccine and methods for making and using thereof include a lipid having the compound structure:

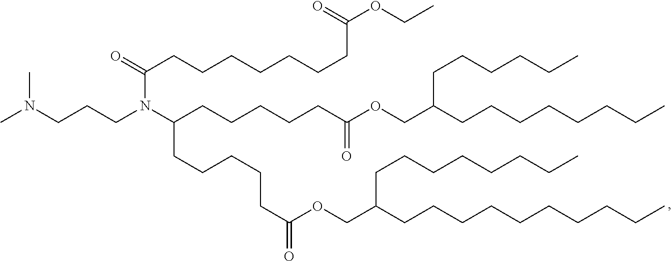 [SMILES: CCCCCCCCCCC(CCCCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof.

In some embodiments, the nucleic acid vaccine and methods for making and using thereof include a lipid having the compound structure:

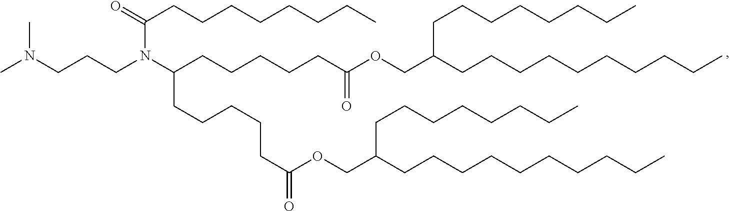 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable lipids for use in the nucleic acid vaccine and methods for making and using thereof include the lipids as described in International Patent Publication WO 2017/075531, which is incorporated herein by reference in its entirety.

In some embodiments, the nucleic acid vaccine and methods for making and using thereof include a lipid of the following formula:

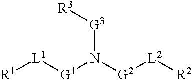

or a pharmaceutically acceptable salt thereof, wherein one of L¹ or L² is —O(C═O)—, —(C═O)O—, —C(═O)—, —O—, —S(O)x, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O) X, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵ C(═O)R⁴; R⁴ is C₁-C₁₂ alkyl; R⁵ is H or C₁-C₆ alkyl; and x is 0, 1 or 2.

Other suitable lipids for use in the nucleic acid vaccine and methods for making and using thereof include the lipids as described in International Patent Publication WO 2017/117528, which is incorporated herein by reference in its entirety. In some embodiments, the nucleic acid vaccine and methods for making and using thereof include a lipid having the compound structure:

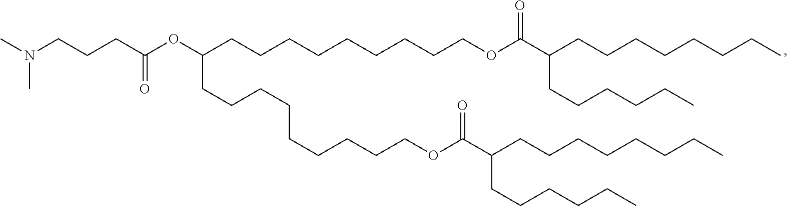 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof.

In some embodiments, the nucleic acid vaccine and methods for making and using thereof include a lipid having the compound structure:

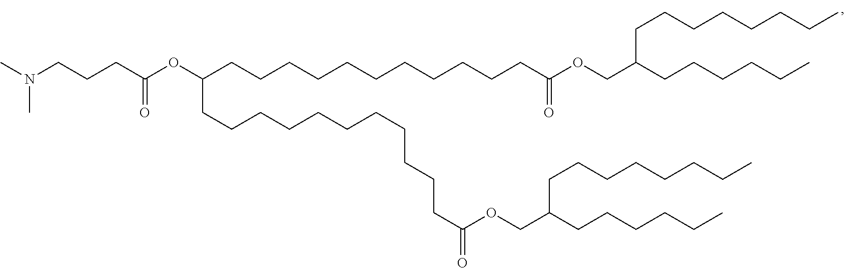 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof.

In some embodiments, the nucleic acid vaccine and methods for making and using thereof include a lipid having the compound structure:

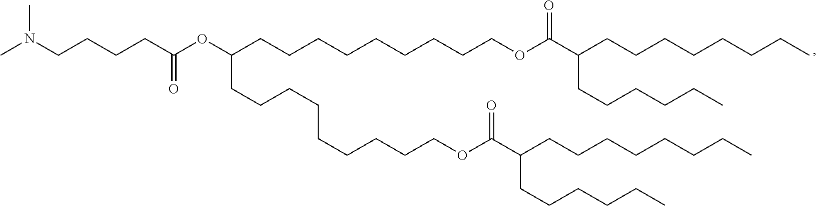 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable lipids for use in the nucleic acid vaccine and methods for making and using thereof include the lipids as described in International Patent Publication WO 2017/049245, which is incorporated herein by reference in its entirety.

In some embodiments, the lipids of the nucleic acid vaccine and methods for making and using thereof include a compound of one of the following formulas:

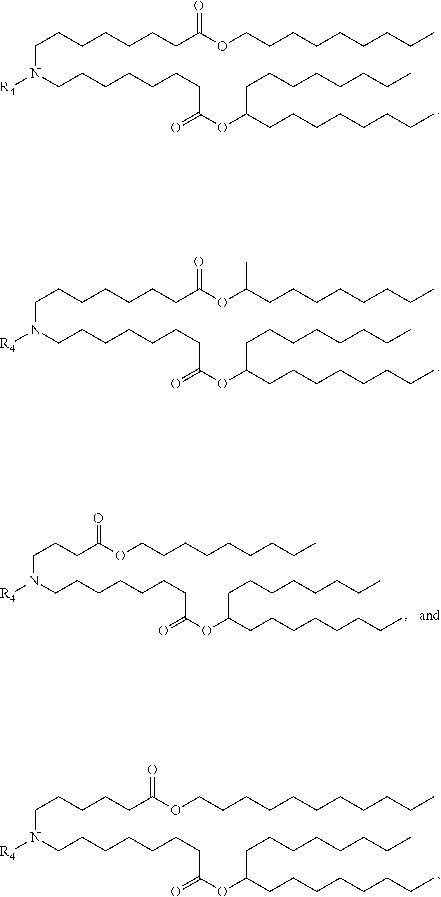

and pharmaceutically acceptable salts thereof. For any one of these our formulas, I is independent selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3.

In certain embodiments, the nucleic acid vaccine and methods for making and using thereof include a lipid having a compound structure of:

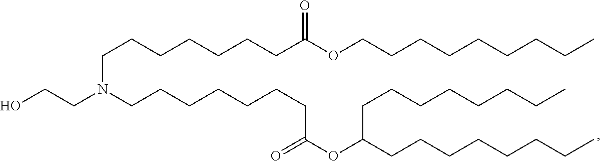 [SMILES: CCCCCCCCCOC(=O)CCCCCCCN(CCO)CCCCCCCC(=O)OC(CCCCCCCC)CCCCCCCC]

and pharmaceutically acceptable salts thereof.

In certain embodiments, the nucleic acid vaccine and methods for making and using thereof include a lipid having a compound structure of:

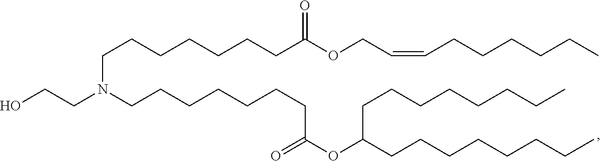 [SMILES: CCCCCC/C=C\COC(=O)CCCCCCCN(CCO)CCCCCCCC(=O)OC(CCCCCCCC)CCCCCCCC]

and pharmaceutically acceptable salts thereof.

In certain embodiments, the nucleic acid vaccine and methods for making and using thereof include a lipid having a compound structure of:

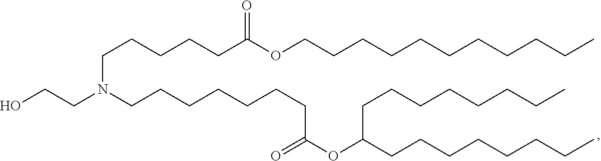 [SMILES: CCCCCCCCCCCOC(=O)CCCCCN(CCO)CCCCCCCC(=O)OC(CCCCCCCC)CCCCCCCC]

and pharmaceutically acceptable salts thereof.

In certain embodiments, the nucleic acid vaccine and methods for making and using thereof include a lipid having a compound structure of:

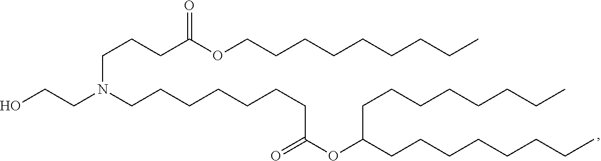 [SMILES: CCCCCCCCCOC(=O)CCCN(CCO)CCCCCCCC(=O)OC(CCCCCCCC)CCCCCCCC]

and pharmaceutically acceptable salts thereof.

Other suitable lipids for use in the nucleic acid vaccine and methods for making and using thereof include the lipids as described in International Patent Publication WO 2017/173054 and WO 2015/095340, each of which is incorporated herein by reference in its entirety. In certain embodiments, the nucleic acid vaccine and methods for making and using thereof include a lipid having a compound structure of:

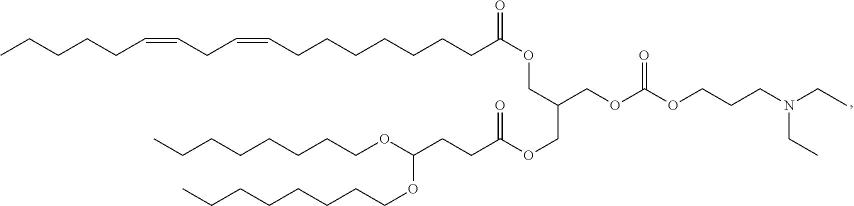 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof.

In certain embodiments, the nucleic acid vaccine and methods for making and using thereof include a lipid having a compound structure of:

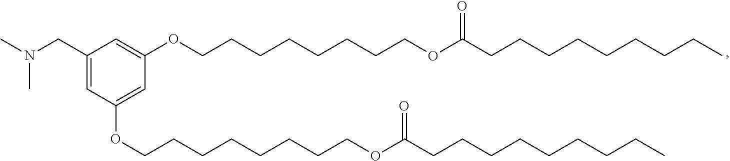 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof.

In certain embodiments, the nucleic acid vaccine and methods for making and using thereof include a lipid having a compound structure of:

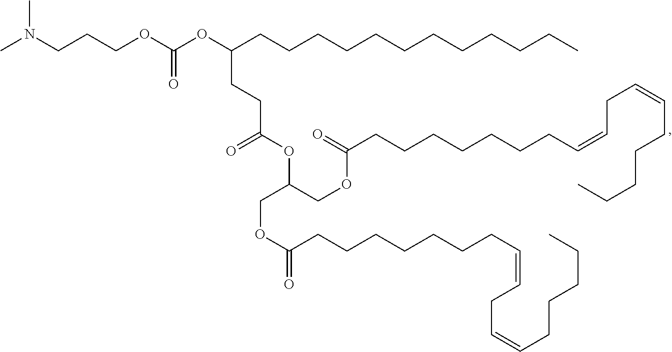 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof.

In certain embodiments, the nucleic acid vaccine and methods for making and using thereof include a lipid having a compound structure of:

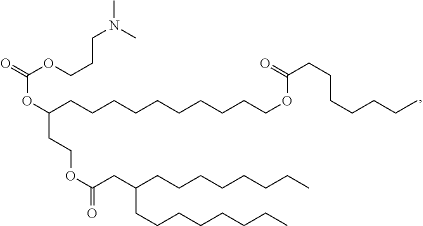 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

In some embodiments, the LPMPs described herein may include a ionizable lipid as described in, may be formulated as described in, or may comprise or be comprised by a composition as described in WO2016118724, WO2016118725, WO2016187531, WO2017176974, WO2018078053, WO2019027999, WO2019036030, WO2019089828, WO2019099501, WO2020072605, WO2020081938, WO2020118041, WO2020146805, or WO2020219876, each of which is incorporated by reference herein in its entirety.

### Other Lipids and Other Agents

The exogenous lipid may be a cell-penetrating agent, may be capable of increasing delivery of a polypeptide by the LPMP to a cell, and/or may be capable of increasing loading (e.g., loading efficiency or loading capacity) of a polypeptide. Further exemplary exogenous lipids include sterols and PEGylated lipids.

The LPMPs can be modified with other components (e.g., lipids, e.g., sterols, e.g., cholesterol; or small molecules) to further alter the functional and structural characteristics of the LPMP. For example, the LPMPs can be further modified with stabilizing molecules that increase the stability of the LPMPs (e.g., for at least one day at room temperature, and/or stable for at least one week at 4° C.).

In some embodiments, the LPMP is modified with a sterol, e.g., sitosterol, sitostanol, β-sitosterol, 7α-hydroxycholesterol, pregnenolone, cholesterol (e.g., ovine cholesterol or cholesterol isolated from plants), stigmasterol, campesterol, fucosterol, or an analog (e.g., a glycoside, ester, or peptide) of any sterol. In some examples, the exogenous sterol is added to the preparation prior to step (b), e.g., mixed with extracted PMP lipids prior to step (b). The exogenous sterol may be added to amount to, e.g., 1%, 5%, 10%, 20%, 30%, 40%, 50%, 60%, 70%, 80%, 90%, or more than 90% (w/w) of total lipids and sterols in the preparation.

In some embodiments, the sterol is cholesterol or sitosterol. In some instances, the LPMPs comprise a molar ratio of least 1%, 5%, 10%, 15%, 20%, 25%, 30%, 35%, 40%, 45%, 50%, 55%, 60%, or more than 60% sterol (e.g., cholesterol or sitosterol), e.g., 1%-10%, 10%-20%, 20%-30%, 30%-40%, 40%-50%, or 50%-60% sterol. In some embodiments, the LPMP comprises a molar ratio of about 35%-50% sterol (e.g., cholesterol or sitosterol), e.g., about 36%, 38.5%, 42.5%, or 46.5% sterol. In some embodiments, the LPMP comprises a molar ratio of about 20%-40% sterol.

In some embodiments, a LPMP that has been modified with a sterol has altered stability (e.g., increased stability) relative to a LPMP that has not been modified with a sterol. In some aspects, a LPMP that has been modified with a sterol has a greater rate of fusion with a membrane of a target cell relative to a LPMP that has not been modified with a sterol.

In some instances, the LPMPs comprise an exogenous lipid and an exogenous sterol.

In some embodiments, the LPMP is modified with a PEGylated lipid. Polyethylene glycol (PEG) length can vary from 1 kDa to 10 kDa; in some aspects, PEG having a length of 2 kDa is used.

In some embodiments, the PEGylated lipid is C14-PEG2k, C18-PEG2k, or DMPE-PEG2k. In some instances, the LPMPs comprise a molar ratio of at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 10%, 20%, 30%, 40%, 50%, or more than 50% PEGylated lipid (e.g., C14-PEG2k, C18-PEG2k, or DMPE-PEG2k), e.g., 0.1%-0.5%, 0.5%-1%, 1%-1.5%, 1.5%-2.5%, 2.5%-3.5%, 3.5%-5%, 5%-10%, 10%-20%, 20%-30%, 30%-40%, or 30%-50% PEGylated lipid. In some embodiments, the LPMP comprises a molar ratio of about 0.1%-10% PEGylated lipid (e.g., C14-PEG2k, C18-PEG2k, or DMPE-PEG2k), e.g., about 1%-3% PEGylated lipid, e.g., about 1.5% or about 2.5% PEGylated lipid. In some embodiments, a LPMP that has been modified with a PEGylated lipid has altered stability (e.g., increased stability) relative to a LPMP that has not been modified with a PEGylated lipid. In some embodiments, a LPMP that has been modified with a PEGylated lipid has altered particle size relative to a LPMP that has not been modified with a PEGylated lipid. In some embodiments, a LPMP that has been modified with a PEGylated lipid is less likely to be phagocytosed than a LPMP that has not been modified with a PEGylated lipid. The addition of PEGylated lipids can also affect stability in GI tract and enhance particle migration through mucus. PEG may be used as a method to attach targeting moieties.

In some embodiments, the LPMPs are modified with an ionizable lipid (e.g., C12-200 or MC3) and one or both of a sterol (e.g., cholesterol or sitosterol) and a PEGylated lipid (e.g., C14-PEG2k, C18-PEG2k, or DMPE-PEG2k).

In some embodiments, the modified LPMPs comprise a molar ratio of about 5%-50% LPMP lipids (e.g., about 10%-20% LPMP lipids, e.g., about 10%, 12.5%, 16%, or 20% LPMP lipids); about 30%-75% ionizable lipids (e.g., about 35% or about 50% ionizable lipids); about 35%-50% sterol (e.g., about 36%, 38.5%, 42.5%, or 46.5% sterol); and about 0.1%-10% PEGylated lipid (e.g., about 1%-3% PEGylated lipid, e.g., about 1.5% or about 2.5% PEGylated lipid).

In some embodiments, the modified LPMPs comprise a molar ratio of about 5%-60% LPMP lipids (e.g., about 10%-20%, 20%-30%, 30%-40%, 40%-50%, or 50%-60% LPMP lipids, e.g., about 10%, 12.5%, 16%, 20%, 30%, 40%, 50%, or 60% LPMP lipids); about 25%-75% ionizable lipids (e.g., about 35% or about 50% ionizable lipids); about 10%-50% sterol (e.g., about 10%, 12.5%, 14%, 16%, 18%, 20%, 36%, 38.5%, 42.5%, or 46.5% sterol); and about 0.1%-10% PEGylated lipid (e.g., about 0.5%-5% PEGylated lipid, e.g., about 1%-3% PEGylated lipid, or about 1.5% or about 2.5% PEGylated lipid).

In some embodiments, the ionizable lipids, LPMP lipids, sterol, and PEGylated lipid comprise about 25%-75%, about 20%-60%, about 10%-45%, and about 0.5%-5%, respectively, of the lipids in the modified PMP.

In some embodiments, the ionizable lipids, LPMP lipids, sterol, and PEGylated lipid comprise about 30%-75%, about 20%-50%, about 10%-45%, and about 1%-5%, respectively, of the lipids in the modified PMP.

In some embodiments, the ionizable lipids, LPMP lipids, sterol, and PEGylated lipid comprise about 35%-75%, about 20%-50%, about 10%-45%, and about 1%-5%, respectively, of the lipids in the modified PMP.

In some embodiments, the ionizable lipids, LPMP lipids, sterol, and PEGylated lipid are formulated at a molar ratio of about 35:50:12.5:2.5.

In some embodiments, the ionizable lipids, LPMP lipids, sterol, and PEGylated lipid are formulated at a molar ratio of about 35:50:11.5:3.5.

In some embodiments, the ionizable lipids, LPMP lipids, sterol, and PEGylated lipid are formulated at a molar ratio of about 35:20:42.5:2.5.

In some embodiments, a LPMP has been modified with an ionizable lipid (and/or cationic lipid) and a sterol and/or a PEGylated lipid more efficiently encapsulates a negatively charged cargo (e.g., a nucleic acid) than a LPMP that has not been modified with an ionizable lipid (and/or cationic lipid) and a sterol and/or a PEGylated lipid. The modified LPMP may have an encapsulation efficiency for the cargo (e.g., nucleic acid, e.g., RNA or DNA) that is at least 5%, 10%, 15%, 20%, 30%, 40%, 50%, 60%, 70%, 80%, 90%, 95%, 98%, 99%, or more than 99%, e.g., may have an encapsulation efficiency of 5%-30%, 30%-50%, 50%-70%, 70%-80%, 80%-90%, 90%-95%, or 95%-100%.

Cell uptake of the modified LPMPs can be measured by a variety of methods known in the art. For example, the LPMPs, or a component thereof, can be labelled with a marker (e.g., a fluorescent marker) that can be detected in isolated cells to confirm uptake.

In some embodiments, a LPMP formulation provided herein comprises two or more different modified LPMPs, e.g., comprises modified LPMPs derived from different unmodified LPMPs (e.g., unmodified LPMPs from two or more different plant sources) and/or comprises modified LPMPs comprising different species and/or different ratios of ionizable lipids, sterols, and/or PEGylated lipids.

In some instances, the organic solvent in which the lipid film is dissolved is dimethylformamide:methanol (DMF:MeOH). Alternatively, the organic solvent or solvent combination may be, e.g., acetonitrile, acetone, ethanol, methanol, dimethylformamide, tetrahydrofuran, 1-buthanol, dimethyl sulfoxide, acetonitrile:ethanol, acetonitrile:methanol, acetone:methanol, methyl tert-butyl ether:propanol, tetrahydrofuran:methanol, dimethyl sulfoxide:methanol, or dimethylformamide:methanol.

The aqueous phase may be any suitable solution, e.g., a citrate buffer (e.g., a citrate buffer having a pH of about 3.2), water, or phosphate-buffered saline (PBS). The aqueous phase may further comprise a nucleic acid (e.g., an siRNA or siRNA precursor (e.g., dsRNA), miRNA or miRNA precursor, mRNA, or plasmid (pDNA)) or a small molecule.

The lipid solution and the aqueous phase may be mixed in the microfluidics device at any suitable ratio. In some examples, aqueous phase and the lipid solution are mixed at a 3:1 volumetric ratio.

LPMPs may optionally include additional agents, e.g., cell-penetrating agents, therapeutic agents, polynucleotides, polypeptides, or small molecules. The LPMPs can carry or associate with additional agents in a variety of ways to enable delivery of the agent to a target plant, e.g., by encapsulating the agent, incorporation of the agent in the lipid bilayer structure, or association of the agent (e.g., by conjugation) with the surface of the lipid bilayer structure. Nucleic acid molecules can be incorporated into the LPMPs either in vivo (e.g., in planta) or in vitro (e.g., in tissue culture, in cell culture, or synthetically incorporated).

### Zeta Potential

The LPMPs comprising an ionizable lipid (e.g., C12-200 or MC3) and optionally a cationic lipid (e.g., DC-cholesterol or DOTAP) may have, e.g., a zeta potential of greater than −30 mV when in the absence of cargo, greater than −20 mV, greater than −5 mV, greater than 0 mV, or about 30 my when in the absence of cargo. In some examples, the LPMP has a negative zeta potential, e.g., a zeta potential of less than 0 mV, less than −10 mV, less than −20 mV, less than −30 mV, less than −40 mV, or less than −50 mV when in the absence of cargo. In some examples, the LPMP has a positive zeta potential, e.g., a zeta potential of greater than 0 mV, greater than 10 mV, greater than 20 mV, greater than 30 mV, greater than 40 mV, or greater than 50 mV when in the absence of cargo. In some examples, the LPMP has a zeta potential of about 0.

The zeta potential of the LPMP may be measured using any method known in the art. Zeta potentials are generally measured indirectly, e.g., calculated using theoretical models from the data obtained using methods and techniques known in the art, e.g., electrophoretic mobility or dynamic electrophoretic mobility. Electrophoretic mobility is typically measured using microelectrophoresis, electrophoretic light scattering, or tunable resistive pulse sensing. Electrophoretic light scattering is based on dynamic light scattering. Typically, zeta potentials are accessible from dynamic light scattering (DLS) measurements, also known as photon correlation spectroscopy or quasi-elastic light scattering.

### Plant EV-Markers

The LPMPs in the nucleic acid vaccine and methods of making and using thereof may have a range of markers that identify the LPMPs as being produced using a plant EV, and/or including a segment, portion, or extract thereof. As used herein, the term “plant EV-marker” refers to a component that is naturally associated with a plant and incorporated into or onto the plant EV in planta, such as a plant protein, a plant nucleic acid, a plant small molecule, a plant lipid, or a combination thereof. Examples of plant EV-markers can be found, for example, in Rutter and Innes, Plant Physiol. 173(1): 728-741, 2017; Raimondo et al., Oncotarget. 6(23): 19514, 2015; Ju et al., Mol. Therapy. 21(7):1345-1357, 2013; Wang et al., Molecular Therapy. 22(3): 522-534, 2014; and Regente et al, J of Exp. Biol. 68(20): 5485-5496, 2017; each of which is incorporated herein by reference.

Additional examples of the suitable plant EV-markers include those described and listed in International Patent Application Publication No. WO 2021/041301, which is incorporated herein by reference in its entirety.

### Loading of Agents (e.g., Nucleic Acids)

The LPMPs are modified to include a therapeutic agent (e.g., a nucleic acid molecule) to form the nucleic acid vaccine. The LPMPs can carry or associate with such agents by a variety of means to enable delivery of the agent to a target organism (e.g., a target animal), e.g., by encapsulating the agent, incorporation of the component in the lipid bilayer structure, or association of the component (e.g., by conjugation) with the surface of the lipid bilayer structure of the LPMP. In some instances, the agent is included in the LPMP formulation, as described herein.

The agent can be incorporated or loaded into or onto the LPMPs by any methods known in the art that allow association, directly or indirectly, between the LPMPs and agent. The agents can be incorporated into the LPMPs by an in vivo method (e.g., in planta, e.g., through production of LPMPs from a transgenic plant that comprises the agent), or in vitro (e.g., in tissue culture, or in cell culture), or both in vivo and in vitro methods.

In some instances, the LPMPs are loaded in vitro. The substance may be loaded onto or into (e.g., may be encapsulated by) the LPMPs using, but not limited to, physical, chemical, and/or biological methods (e.g., in tissue culture or in cell culture). For example, the agent may be introduced into LPMPs by one or more of electroporation, sonication, passive diffusion, stirring, lipid extraction, or extrusion. In some instances, the agent is incorporated into the LPMP using a microfluidic device, e.g., using a method in which LPMP lipids are provided in an organic phase, the heterologous functional agent is provided in an aqueous phase, and the organic and aqueous phases are combined in the microfluidics device to produce a LPMP comprising the heterologous functional agent. Loaded LPMPs can be assessed to confirm the presence or level of the loaded agent using a variety of methods, such as HPLC (e.g., to assess small molecules), immunoblotting (e.g., to assess proteins); and/or quantitative PCR (e.g., to assess nucleotides). However, it should be appreciated by those skilled in the art that the loading of a substance of interest into LPMPs is not limited to the above-illustrated methods.

In some instances, the agent can be conjugated to the LPMP, in which the agent is connected or joined, indirectly or directly, to the LPMP. For instance, one or more agents can be chemically linked to a LPMP, such that the one or more agents are joined (e.g., by covalent or ionic bonds) directly to the lipid bilayer of the LPMP. In some instances, the conjugation of various agents to the LPMPs can be achieved by first mixing the one or more agents with an appropriate cross-linking agent (e.g., N-ethylcarbo-diimide (“EDC”), which is generally utilized as a carboxyl activating agent for amide bonding with primary amines and also reacts with phosphate groups) in a suitable solvent. After a period of incubation sufficient to allow the agent to attach to the cross-linking agent, the cross-linking agent/agent mixture can then be combined with the LPMPs and, after another period of incubation, subjected to a sucrose gradient (e.g., and 8, 30, 45, and 60% sucrose gradient) to separate the free agent and free LPMPs from the agent conjugated to the LPMPs. As part of combining the mixture with a sucrose gradient, and an accompanying centrifugation step, the LPMPs conjugated to the agent are then seen as a band in the sucrose gradient, such that the conjugated LPMPs can then be collected, washed, and dissolved in a suitable solution for use as described herein.

In some instances, the LPMPs are stably associated with the agent prior to and following delivery of the LPMP, e.g., to a plant. In other instances, the LPMPs are associated with the agent such that the agent becomes dissociated from the LPMPs following delivery of the LPMP, e.g., to a plant.

The LPMPs can be loaded or the LPMP can be formulated with various concentrations of the agent, depending on the particular agent or use. For example, in some instances, the LPMPs are loaded or the LPMP is formulated such that the LPMP formulation disclosed herein includes about 0.001, 0.01, 0.1, 1.0, 2, 3, 4, 5, 6, 7, 8, 9, 10, 15, 20, 30, 40, 50, 60, 70, 80, 90, or 95 (or any range between about 0.001 and 95) or more wt % of an agent. In some instances, the LPMPs are loaded or the LPMP is formulated such that the LPMP formulation includes about 95, 90, 80, 70, 60, 50, 40, 30, 20, 15, 10, 9, 8, 7, 6, 5, 4, 3, 2, 1.0, 0.1, 0.01, 0.001 (or any range between about 95 and 0.001) or less wt % of an agent. For example, the LPMP formulation can include about 0.001 to about 0.01 wt %, about 0.01 to about 0.1 wt %, about 0.1 to about 1 wt %, about 1 to about 5 wt %, or about 5 to about 10 wt %, about 10 to about 20 wt % of the agent. In some instances, the LPMP can be loaded or the LPMP is formulated with about 1, 5, 10, 50, 100, 200, or 500, 1,000, 2,000 (or any range between about 1 and 2,000) or more μg/ml of an agent. A LPMP of the invention can be loaded or a LPMP can be formulated with about 2,000, 1,000, 500, 200, 100, 50, 10, 5, 1 (or any range between about 2,000 and 1) or less μg/ml of an agent.

In some instances, the LPMPs are loaded or the LPMP is formulated such that the LPMP formulation disclosed herein includes at least 0.001 wt %, at least 0.01 wt %, at least 0.1 wt %, at least 1.0 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 30 wt %, at least 40 wt %, at least 50 wt %, at least 60 wt %, at least 70 wt %, at least 80 wt %, at least 90 wt %, or at least 95 wt % of an agent. In some instances, the LPMP can be loaded or the LPMP can be formulated with at least 1 μg/ml, at least 5 μg/ml, at least 10 μg/ml, at least 50 μg/ml, at least 100 μg/ml, at least 200 μg/ml, at least 500 μg/ml, at least 1,000 μg/ml, at least 2,000 μg/ml of an agent.

In some instances, the LPMP is formulated with the agent by suspending the LPMPs in a solution comprising or consisting of the agent, e.g., suspending or resuspending the LPMPs by vigorous mixing. The agent (e.g., cell-penetrating agent, e.g., nucleic acids, enzyme, detergent, ionic, fluorous, or zwitterionic liquid, or ionizable lipid may comprise, e.g., less than 1% or at least 1%, 5%, 10%, 20%, 30%, 40%, 50%, 60%, 70%, 80%, 90%, or 100% of the solution.

### Pharmaceutical Formulations

The modified LPMPs are formulated into pharmaceutical compositions (i.e., a nucleic acid vaccine composition), e.g., for administration to an animal (e.g., a human). The pharmaceutical composition may be administered to an animal (e.g., human) with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed.

In some embodiments, the dose is 0.005 mg/kg, 0.01 mg/kg, 0.05 mg/kg, 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or more.

In some embodiments, the vaccine is administered once, twice, three times, four times, or more.

The LPMP/nucleic acid vaccine may be formulated for e.g., oral administration, intranasal, intravenous administration (e.g., injection or infusion), intramuscular, or subcutaneous administration to an animal. For injectable formulations, various effective pharmaceutical carriers are known in the art (See, e.g., Remington: The Science and Practice of Pharmacy, 22^{nd} ed., (2012) and ASHP Handbook on Injectable Drugs, 18^{th} ed., (2014)).

Suitable pharmaceutically acceptable carriers and excipients are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol.

The LPMP/nucleic acid vaccine may be formulated according to conventional pharmaceutical practice. The concentration of the compound in the formulation will vary depending upon a number of factors, including the dosage of the active agent (e.g., LPMPs and nucleic acids) to be administered, and the route of administration.

For oral administration to an animal, the LPMP/nucleic acid vaccine can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium). The compositions disclosed herein may also further include an immediate-release, extended release or delayed-release formulation.

For parenteral administration to an animal, the LPMP/nucleic acid vaccines may be formulated in the form of liquid solutions or suspensions and administered by a parenteral route (e.g., subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), and F-12 medium). Formulation methods are known in the art, see e.g., Gibson (ed.) Pharmaceutical Preformulation and Formulation (2nd ed.) Taylor & Francis Group, CRC Press (2009).

### Polynucleotides

The LPMP/nucleic acid vaccine includes one or more nucleic acid molecules, e.g., polynucleotides, which encode one or more wild type or engineered proteins, peptides, or polypeptides. Exemplary polynucleotides, e.g., polynucleotide constructs, include antigen—encoding RNA polynucleotides, e.g., mRNAs.

Examples of polypeptides that can be used herein can include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

Polypeptides included herein may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

The LPMP/nucleic acid vaccine may include any number or type (e.g., classes) of polypeptides, such as at least about any one of 1 polypeptide, 2, 3, 4, 5, 10, 15, 20, or more polypeptides. A suitable concentration of each polypeptide in the LPMP/nucleic acid vaccine depends on factors such as efficacy, stability of the polypeptide, number of distinct polypeptides in the formulation, and methods of application of the formulation. In some instances, each polypeptide in a liquid formulation is from about 0.1 ng/mL to about 100 mg/mL. In some instances, each polypeptide in a solid formulation is from about 0.1 ng/g to about 100 mg/g.

### Nucleic Acids Encoding Peptides

In some instances, the LPMP/nucleic acid vaccine include a heterologous nucleic acid encoding a polypeptide. Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range therebetween.

The LPMP/nucleic acid vaccine may also include active variants of a nucleic acid sequence of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the invention includes an active polypeptide encoded by a nucleic acid variant as described herein. In some instances, the active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Certain methods for expressing a nucleic acid encoding a protein may involve expression in cells, including insect, yeast, plant, bacteria, or other cells under the control of appropriate promoters. Expression vectors may include nontranscribed elements, such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instance, provided is a composition to alter expression of one or more proteins, e.g., proteins that affect activity, structure, or function. Expression of the one or more proteins may be restricted to a specific location(s) or widespread throughout the organism. mRNA

The LPMP/nucleic acid vaccine may include a mRNA molecule, e.g., a mRNA molecule encoding a polypeptide. The mRNA molecule can be synthetic and modified (e.g., chemically). The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some instances, the modified RNA agent of interest described herein has modified nucleosides or nucleotides. Such modifications are known and are described, e.g., in WO 2012/019168. Additional modifications are described, e.g., in WO 2015/038892; WO 2015/038892; WO 2015/089511; WO 2015/196130; WO 2015/196118 and WO 2015/196128 A2, which are herein incorporated by reference in their entirety.

In some instances, the modified RNA encoding a polypeptide of interest has one or more terminal modification, e.g., a 5′ cap structure and/or a poly-A tail (e.g., of between 100-200 nucleotides in length). The 5′ cap structure may be selected from the group consisting of CapO, CapI, ARCA, inosine, NI-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5′ UTR including at least one Kozak sequence, and a 3′ UTR. Such modifications are known and are described, e.g., in WO 2012/135805 and WO 2013/052523, which are incorporated herein by reference in their entirety. Additional terminal modifications are described, e.g., in WO 2014/164253 and WO 2016/011306, WO 2012/045075, and WO 2014/093924, which are incorporated herein by reference in their entirety. Chimeric enzymes for synthesizing capped RNA molecules (e.g., modified mRNA) which may include at least one chemical modification are described in WO 2014/028429, which is incorporated herein by reference in its entirety.

In some instances, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667 and WO 2013/151736, which are incorporated herein by reference in their entirety. Methods of purification include purifying an RNA transcript including a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified mRNA sample to DNAse treatment (WO 2014/152030).

Formulations of modified RNAs are known and are described, e.g., in WO 2013/090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; Tables 6, 185 and 186 of International Publication No WO 2013/151667; which are incorporated herein by reference in their entirety. Any of the foregoing may be synthesized as an IVT polynucleotide, chimeric polynucleotide or a circular polynucleotide, and each may include one or more modified nucleotides or terminal modifications.

### Inhibitory RNA

In some instances, the LPMP/nucleic acid vaccine includes an inhibitory RNA molecule, e.g., that acts via the RNA interference (RNAi) pathway. In some instances, the inhibitory RNA molecule decreases the level of gene expression in a plant and/or decreases the level of a protein in the plant. In some instances, the inhibitory RNA molecule inhibits expression of a plant gene. For example, an inhibitory RNA molecule may include a short interfering RNA or its precursor, short hairpin RNA, and/or a microRNA or its precursor that targets a gene in the plant. Certain RNA molecules can inhibit gene expression through the biological process of RNA interference (RNAi). RNAi molecules include RNA or RNA-like structures typically containing 15-50 base pairs (such as about 18-25 base pairs) and having a nucleobase sequence identical (or complementary) or nearly identical (or substantially complementary) to a coding sequence in an expressed target gene within the cell. RNAi molecules include, but are not limited to short interfering RNAs (siRNAs), double-strand RNAs (dsRNA), short hairpin RNAs (shRNA), meroduplexes, dicer substrates, and multivalent RNA interference (U.S. Pat. Nos. 8,084,599 8,349,809, 8,513,207 and 9,200,276, which are incorporated herein by reference in their entirety). The inhibitory RNA molecule can be chemically synthesized or transcribed in vitro.

Additional examples of the inhibitory RNA molecules include those described in details in International Patent Application Publication No. WO 2021/041301, which is incorporated herein by reference in its entirety.

### Gene Editing

In some instances, the LPMP/nucleic acid vaccines may include a component of a gene editing system. For example, the agent may introduce an alteration (e.g., insertion, deletion (e.g., knockout), translocation, inversion, single point mutation, or other mutation) in a gene in the plant. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al., Trends Biotechnol. 31(7):397-405, 2013.

Additional descriptions about the component and process of the gene editing system may be found in International Patent Application Publication No. WO 2021/041301, which is incorporated herein by reference in its entirety.

### Nucleic Acid Vaccine For Viral Infection

The LPMP/nucleic acid vaccine comprises one or more polynucleotides (e.g., mRNA) encoding one or more antigenic polypeptides to combat various viral infections. The one or more polynucleotides (e.g., mRNA) encode one or more antigenic polypeptides derived from an infectious agent that causes an infectious disease, disorder, or condition, e.g., a viral infection caused by an RNA virus.

In some embodiments, the polynucleotide (e.g., mRNA) in the LPMP/nucleic acid vaccine encodes one or more wild type or engineered antigens (or an antibody to an antigen) of the various types and strains of virus as described below.

### Viral Infection.

The LPMP/nucleic acid vaccine may be suitable to combat the infectious diseases, disorders, or conditions associated with viral infections including, but not limited to, acute febrile pharyngitis, pharyngoconjunctival fever, epidemic keratoconjunctivitis, infantile gastroenteritis, coxsackie infections, infectious mononucleosis, burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, cytomegalic inclusion disease, kaposi sarcoma, multicentric castleman disease, primary effusion lymphoma, AIDS, influenza, reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, poliomyelitis, rabies, bronchiolitis, pneumonia, influenza-like syndrome, severe bronchiolitis with pneumonia, german measles, congenital rubella, varicella, and herpes zoster.

Exemplary viral infectious agents include, but are not limited to, a strain of virus selected from the group consisting of: adenovirus; Herpes simplex, type 1; Herpes simplex, type 2; encephalitis virus, papillomavirus, Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabiá virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Hantavirus, Middle East Respiratory Coronavirus; Chikungunya virus or Banna virus.

The infectious agent may be a strain of virus selected from the group consisting of the virus from the following table.

Adenovirus
Banna virus
BK virus
Chikungunya Virus
Coltivirus
coxsackievirus
Crimean-Congo hemorrhagic fever virus
Dengue virus
Ebola virus
Encephalitis virus
Japanese encephalitis virus
Eastern equine encephalitis
Epstein-barr virus
Guanarito virus
Hanta virus
Herpes simplex, type 1
Herpes simplex, type 2
Human herpes virus, type 8
Herpes zoster (Varicella-zoster; Shingles) virus
Hepatitis A virus
Hepatitis B virus
Hepatitis C virus
Hepatitis D
Hepatitis E virus
Hendra virus
Human astrovirus
Human bocavirus
Human cytomegalovirus
Human Enterovirus
Human Enterovirus 68
Human Enterovirus 71
Human metapneumovirus
Human Immunodeficiency virus (HIV)
Influenza virus
Parainfluenza virus
JC virus
Junin virus
Lassa virus
Machupo virus
Marburg virus
Measles virus
Mumps virus
Nipah virus
Norwalk virus
Orbivirus
papillomavirus
Human papillomavirus (HPV)
Parvovirus B19
polio virus
Rabies virus
Rhinovirus
Rotavirus
Rubella virus
Respiratory syncytial virus (RSV)
Severe acute respiratory syndrome (SARS) virus
Middle East Respiratory Syndrome Corona (MERS) Virus
SARS-CoV-2
Smallpox
Sabia virus
Vesicular exanthernavirus
West Nile virus
Yellow Fever virus
Zika virus

Other suitable viral infections and viral infectious agents are described in U.S. Patent Application Publication No. US2019/0015501 and U.S. Pat. No. 11,007,260, both of which are incorporated by reference in their entirety.

### A Mosquito-Borne Virus

Dengue. In some embodiments, the polynucleotide (e.g., mRNA) in the LPMP/nucleic acid vaccine encodes a strain of dengue virus (a flavi virus). In some embodiments, the polynucleotide (e.g., mRNA) encodes the E protein domain III (DENV1-4 tandem mRNA), the E protein domain I/II hinge region (DENV1-4 individual mRNAs), the prM protein (DENV1-4 tandem or individual mRNAs) and the C protein (DENV1-4 tandem or single mRNAs).

Chikungunya virus. In some embodiments, the polynucleotide (e.g., mRNA) in the LPMP I nucleic acid vaccine encodes a strain of Chikungunya virus. In some embodiments, the antigenic polypeptide encodes Chikungunya envelope and/or capsid antigenic polypeptide selected from the group consisting of C, E1, E2, E3, 6K, and C-E3-E2-6K-E1.

In some embodiments, the polynucleotide (e.g., mRNA) in the LPMP/nucleic acid vaccine encodes a Chikungunya polypeptide selected from the following strains and isolates: TA53, SA76, UG82, 37997, IND-06, Ross, S27, M-713424, E1-A226V, E1-T98, IND-63-WB1, Gibbs 63-263, TH35, 1-634029, AF15561, IND-73-MH5, 653496, C0392-95, P0731460, MY0211MR/06/BP, SV0444-95, K0146-95, TSI-GSD-218-VR1, TSI-GSD-218, M127, M125, 6441-88, MY003IMR/06/BP, MY0021MR/06/BP, TR206/H804187, MY/06/37348, MY/06/37350, NC/2011-568, 1455-75, RSU1, 0706aTw, InDRE51CHIK, PR-S4, AMA2798/H804298, Hu/85/NR/001, PhH15483, 0706aTw, 0802aTw, MY0191MR/06/BP, PR-S6, PER160/H803609, 99659, JKT23574, 0811 aTw, CHIK/SBY6/10, 2001908323-BDG E1, 2001907981-BDG E1, 2004904899-BDG E1, 2004904879-BDG E1, 2003902452-BDG E1, DH 130003, 0804aTw, 2002918310-BDG E1, JC2012, chik-sy, 3807, 3462, Yap 13-2148, PR-S5, 0802aTw, MY019IMR/06/Bp, 0706aTw, PhH15483, Hu/85/NR/001, CHIKV-13-112A, InDRE 4CHIK, 0806aTw, 0712aTw, 3412-78, Yap 13-2039, LEIV-CHIKV/Moscow/1, DH130003, and 20039.

Zika virus. In some embodiments, the polynucleotide (e.g., mRNA) in the LPMP/nucleic acid vaccine encodes a strain of Zika virus (a flavi virus). In some embodiments, the polynucleotide (e.g., mRNA) in the LPMP/nucleic acid vaccine encodes a ZIKV polypeptide from a ZIKV serotype selected from the group consisting of MR 766, SPH2015, and ACD75819.

Venezuelan equine encephalitis (VEE) virus. In some embodiments, the polynucleotide (e.g., mRNA) in the LPMP/nucleic acid vaccine encodes a strain of VEE virus.

The polynucleotide (e.g., mRNA) in the LPMP/nucleic acid vaccine can encode additional types of virus and strains of a mosquito-borne virus, or fragments thereof, as those described in U.S. Pat. No. 11,007,260, which is incorporated herein by reference in its entirety.

### Influenza

In some embodiments, the polynucleotide (e.g., mRNA) in the LPMP/nucleic acid vaccine encodes a strain of an influenza virus. In some embodiments, the polynucleotide (e.g., mRNA) in the LPMP/nucleic acid vaccine encodes a strain of Influenza A or Influenza B or combinations thereof.

In some embodiments, the strain of Influenza A or Influenza B is associated with birds, pigs, horses, dogs, humans or non-human primates.

In some embodiments, the polynucleotide (e.g., mRNA) in the LPMP/nucleic acid vaccine encodes encodes a hemagglutinin protein or fragment thereof. In some embodiments, the hemagglutinin protein is H1, H2, H3, H4, H5, H6, H7, H8, H9, H10, H11, H12, H13, H14, H15, H16, H17, H18, or a fragment thereof. In some embodiments, the hemagglutinin protein does not comprise a head domain (HA1). In some embodiments, the hemagglutinin protein comprises a portion of the head domain (HA1). In some embodiments, the hemagglutinin protein does not comprise a cytoplasmic domain. In some embodiments, the hemagglutinin protein comprises a portion of the cytoplasmic domain.

In some embodiments, the polynucleotide (e.g., mRNA) in the LPMP/nucleic acid vaccine encodes encodes a truncated hemagglutinin protein. In some embodiments, the truncated hemagglutinin protein comprises a portion of the transmembrane domain. In some embodiments, the virus is selected from the group consisting of H1N1, H3N2, H5N1, H7N9, and H10N8.

The polynucleotide (e.g., mRNA) in the LPMP/nucleic acid vaccine can encode additional types of virus and strains of an influenza virus, or fragments thereof, as those described in U.S. Patent Application Publication No. 2019/0015501, which is incorporated herein by reference in its entirety.

### Coronavirus

Betacoronavirus. In some embodiments, the polynucleotide (e.g., mRNA) in the LPMP/nucleic acid vaccine encodes a peptide/protein comprising a spike protein (S) of a betacoronavirus (BetaCoV), or a fragment or subunit thereof.

In some embodiments, the polynucleotide (e.g., mRNA) in the LPMP/nucleic acid vaccine comprises an open reading frame encoding a peptide/protein comprising a spike protein (S) of a betacoronavirus (BetaCoV), or a fragment or subunit thereof.

The polynucleotide (e.g., mRNA) in the LPMP/nucleic acid vaccine can encode different types of betacoronavirus and strains of a betacoronavirus, or fragments thereof, as those described in U.S. Pat. No. 10,933,127, which is incorporated herein by reference in its entirety.

Middle East respiratory syndrome coronavirus. In some embodiments, the polynucleotide (e.g., mRNA) in the LPMP/nucleic acid vaccine encodes a peptide/protein comprising a spike protein (S), a spike S1 fragment (S1), an envelope protein (E), a membrane protein (M), and/or a nucleocapsid protein (N) of a MERS coronavirus, or a fragment or variant of any one of these proteins.

The polynucleotide (e.g., mRNA) in the LPMP/nucleic acid vaccine can encode different strains of an MERS coronavirus, or fragments thereof, as those described in U.S. Patent Application Publication No. US2019/0351048, which is incorporated herein by reference in its entirety.

### SARS-CoV-2

In some embodiments, the polynucleotide (e.g., mRNA) in the LPMP/nucleic acid vaccine encodes one or more wild type or engineered antigens (or an antibody to an antigen) of SARS-CoV-2.

In some embodiments, the open reading frame of the polynucleotide (e.g., mRNA) in the LPMP/nucleic acid vaccine encodes one or more wild type or engineered antigens (or an antibody to an antigen) of SARS-CoV-2. In some embodiments, the open reading frame is codon-optimized.

In some embodiments, the polynucleotide (e.g., mRNA) in the LPMP/nucleic acid vaccine encodes a peptide/protein comprising a spike protein (S), a membrane (M) protein, an envelope (E) protein, and/or a nucleocapsid (NC) protein of a SARS-CoV-2 virus, or a fragment or variant thereof.

In some embodiments, the polynucleotide (e.g., mRNA) in the LPMP/nucleic acid vaccine encodes a peptide/protein comprising a spike protein (S) of a SARS-CoV-2 virus, or a fragment or variant thereof. In some embodiments, the polynucleotide (e.g., mRNA) in the LPMP/nucleic acid vaccine codes a peptide/protein comprising at least one or two domains a spike protein (S) of a SARS-CoV-2 virus, and less than the full length spike protein.

In some embodiments, the polynucleotide (e.g., mRNA) in the LPMP/nucleic acid vaccine comprises an open reading frame (ORF) that encodes a SARS-CoV-2 spike (S) protein having a double proline stabilizing mutation.

In some embodiments, the polynucleotide (e.g., mRNA) in the LPMP/nucleic acid vaccine (or its open reading frame) encodes a strain or variant of SARS-CoV-2 virus selected from the group consisting of:
alpha (lineage B.1.1.7, Q.1-Q.8),
beta (lineage B.1.351, B.1.351.2, B.1.351.3),
delta
gamma (lineage P.1, P.1.1, P.1.2)
epsilon (lineage B.1.427, B.1.429)
eta (lineage B.1.525)
iota (lineage B.1.526)
kappa (lineage B.1.617.1)
B.1.617.3
Lambda (lineage C.37)
mu (lineage B.1.621, B.1.621.1)
zeta (lineage P.2).
omicron

In some embodiments, the polynucleotide (e.g., mRNA) in the LPMP/nucleic acid vaccine (or its open reading frame) encodes a SARS-CoV-2 spike (S) protein and/or RBD, or fragments thereof.

In some embodiments, the S antigen and/or RBD antigen fragment thereof comprises one or more mutations within the RBD selected from the group consisting of: K417N or K417T, N439N, N440K, G446V, L452R, Y453F, S477G or S477N, E484Q or E484K, F490S, N501S or N501Y, D614G, Q677P or Q677H, P681H or P681R.

In some embodiments, the polynucleotide (e.g., mRNA) in the LPMP/nucleic acid vaccine (or its open reading frame) encodes a SARS-CoV-2 spike (S) protein and/or RBD, or fragments thereof.

In some embodiments, the S antigen comprises a mutation which stabilizes the Spike trimer, including e.g., the K986P and V987P mutations (S-2P variant) and other proline substitutions, in particular F817P, A892P, A899P and A942P, which can be combined together to obtain a multiple proline variant, in particular hexaproline variant (HexaPro).

In some embodiments, the polynucleotide (e.g., mRNA) in the LPMP/nucleic acid vaccine (or its open reading frame) encodes a SARS-CoV-2 spike (S) protein and/or RBD, or fragments thereof.

In some embodiments, the S antigen comprises one or more mutations selected from the group consisting of: the substitutions L18F, T20N, P26S, D80A, D138Y, R190S, D215G, A570D, D614G, H655Y, P681H, A701V, T7161, S982A, T10271, D1118H and V1176F; and the deletions delta 69-70, delta 144, delta 242-244, and delta 246-252.

In some embodiments, the polynucleotide (e.g., mRNA) in the LPMP/nucleic acid vaccine (or its open reading frame) encodes a SARS-CoV-2 spike (S) protein and/or RBD, or fragments thereof.

In some embodiments, the S antigen or RBD antigen fragment thereof comprises the following mutations: N501Y; E484K and N501Y; K417T or K417N, E484K and N501Y; K417N, N439N, Y453F, S477N, E484K, F490S, and N501Y; K417N, N439N, L452R, S477N, E484K, F490S, and N501Y.

Additional mutations may be found in WO 2021/154763A1, which is incorporated by reference in its entirety.

### Nucleic Acid Sequences

In some embodiments, the antigenic polypeptide encoded by the polynucleotide is a corona virus, or a fragment or subunit thereof. In some embodiments, the antigenic polypeptide is spike protein (S) of a MERS virus (MERS-CoV), a SARS virus (SARS-CoV), or a fragment or subunit thereof.

In some embodiments, the antigenic polypeptide is a SARS virus, or a fragment or subunit thereof. The antigenic polypeptide may be a SARS-CoV-2 spike protein or a SARS-CoV-2 spike glycoprotein.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA.

In some embodiments, the polynucleotide encodes a coronavirus antigen variant (e.g., variant trimeric spike protein, such as a stabilized prefusion spike protein). Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence that is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

In some embodiments, the antigenic polypeptide is a structural protein. In some embodiments, the antigenic polypeptide is a spike protein, an envelope protein, a nucleocapsid protein, or a membrane protein. In some embodiments, the antigenic polypeptide is a stabilized prefusion spike protein. In some embodiments, the mRNA comprises an open reading frame that encodes a variant trimeric spike protein. The trimeric spike protein, for example, may comprise a stabilized prefusion spike protein. In some embodiments, the stabilized prefusion spike protein a double proline (S2P) mutation.

In some embodiments, the polynucleotide (e.g., mRNA) having an open reading frame (ORF) encoding a coronavirus antigen (e.g., variant trimeric spike protein, such as a stabilized prefusion spike protein). In some embodiments, the RNA (e.g., mRNA) further comprises a 5′ UTR, 3′ UTR, a poly(A) tail and/or a 5′ cap analog.

In some embodiments, the mRNA comprises a 5′ untranslated region (UTR) and/or a 3′ UTR.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U”

In some embodiments, the mRNA is derived from (a) a DNA molecule; or (b) an RNA molecule. In the mRNA, T is optionally substituted with U.

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. The sequences disclosed herein may further comprise additional elements, e.g., 5′ and 3′ UTRs, but that those elements, unlike the ORF, need not necessarily be present in a polynucleotide of the present disclosure.

### Stabilizing Elements

Naturally occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, the polynucleotide has an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Vims Capping Enzyme to generate the “Cap 0” structure: m7G(5′)ppp(5′)G (New England BioLabs, Ipswich, MA). Cap 1 structure may be generated using both Vaccinia Vims Capping Enzyme and a 2′-o methyl-transferase to generate m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, the polynucleotide includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the polynucleotide (e.g., mRNA) includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, b-Galactosidase, EGFP), or a marker or selection protein (e.g. alpha-Globin, Galactokinase and Xanthine:guanine phosphoribosyl transferase (GPT)).

In some embodiments, the polynucleotide (e.g., mRNA) includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence. In some embodiments, an RNA (e.g., mRNA) does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

The polynucleotide (e.g., mRNA) may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, the polynucleotide (e.g., mRNA) has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, the polynucleotide (e.g., mRNA) has an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and in prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane. A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid of the disclosure. In some embodiments, the signal peptide may comprise those described in WO 2021/154763, which is incorporated by reference in its entirety.

### Fusion Proteins

In some embodiments, the polynucleotide (e.g., mRNA) encodes an antigenic fusion protein.

Thus, the encoded antigen or antigens may include two or more proteins (e.g., protein and/or protein fragment) joined together. Alternatively, the protein to which a protein antigen is fused does not promote a strong immune response to itself, but rather to the coronavirus antigen. Antigenic fusion proteins, in some embodiments, retain the functional property from each original protein.

### Scaffold Moieties

The polynucleotide (e.g., mRNA), in some embodiments, encodes fusion proteins that comprise coronavirus antigens linked to scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of −22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 A and 360 A diameter, corresponding to 180 or 240 protomers. In some embodiments, the coronavims antigen is fused to HBsAG or HBcAG to facilitate self-assembly of nanoparticles displaying the coronavims antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Fawson D. M. et al. Nature. 1991; 349:541-544). Ferritin self-assembles into nanoparticles with robust thermal and chemical stability. Thus, the ferritin nanoparticle is well suited to carry and expose antigens.

Fumazine synthase (FS) is also well suited as a nanoparticle platform for antigen display. FS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The FS monomer is 150 amino acids long, and consists of beta-sheets along with tandem alpha-helices flanking its sides. A number of different quaternary structures have been reported for FS, illustrating its morphological versatility: from homopentamers up to symmetrical assemblies of 12 pentamers forming capsids of 150 A diameter. Even FS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, the polynucleotide encodes a coronavims antigen (e.g., SARS-CoV-2 S protein) fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, the polynucleotide (e.g., mRNA) encodes more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2 A peptides, has been described in the art (see for example, Kim, J. H. et al. (2011) PLoS ONE 6:el8556). In some embodiments, the linker is an F2A linker. In some embodiments, the fusion protein contains three domains with intervening linkers, having the structure: domain-linker-domain-linker-domain.

Cleavable linkers known in the art may be used in connection with the disclosure. Exemplary such linkers include F2A linkers, T2A linkers, P2A linkers, E2A linkers (See, e.g., WO2017/127750, which is incorporated herein by reference in its entirety). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a coronavirus antigen). In some embodiments, a codon optimized sequence shares less than 90% sequence identity to a naturally occurring or wild-type sequence (e.g., a naturally occurring or wild-type mRNA sequence encoding a coronavirus antigen). In some embodiments, a codon optimized sequence shares less than 85% sequence identity to a naturally occurring or wild-type sequence (e.g., a naturally occurring or wild-type mRNA sequence encoding a coronavirus antigen). In some embodiments, a codon optimized sequence shares less than 80% sequence identity to a naturally occurring or wild-type sequence (e.g., a naturally occurring or wild-type mRNA sequence encoding a coronavirus antigen). In some embodiments, a codon optimized sequence shares less than 75% sequence identity to a naturally occurring or wild-type sequence (e.g., a naturally occurring or wild-type mRNA sequence encoding a coronavirus antigen).

In some embodiments, a codon optimized sequence shares between 65% and 85% (e.g., between about 67% and about 85% or between about 67% and about 80%) sequence identity to a naturally occurring or wild-type sequence (e.g., a naturally occurring or wild-type mRNA sequence encoding a coronavirus antigen). In some embodiments, a codon optimized sequence shares between 65% and 75% or about 80% sequence identity to a naturally occurring or wild-type sequence (e.g., a naturally occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence. When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, the polynucleotide (e.g., mRNA) is not chemically modified and comprises the standard ribonucleotides consisting of adenosine, guanosine, cytosine and uridine. In some embodiments, nucleotides and nucleosides of the polynucleotide (e.g., mRNA) comprise standard nucleoside residues such as those present in transcribed RNA (e.g. A, G, C, or U). In some embodiments, nucleotides and nucleosides of the polynucleotide (e.g., mRNA) comprise standard deoxyribonucleosides such as those present in DNA (e.g. dA, dG, dC, or dT).

### Chemical Modifications

The polynucleotide (e.g., mRNA) comprises, in some embodiments, an RNA having an open reading frame encoding a coronavirus antigen, wherein the nucleic acid comprises nucleotides and/or nucleosides that can be standard (unmodified) or modified as is known in the art. In some embodiments, nucleotides and nucleosides of the polynucleotide (e.g., mRNA) comprise modified nucleotides or nucleosides. Such modified nucleotides and nucleosides can be naturally occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

The nucleic acids of the polynucleotide (e.g., mRNA) can comprise standard nucleotides and nucleosides, naturally-occurring nucleotides and nucleosides, non-naturally-occurring nucleotides and nucleosides, or any combination thereof.

Nucleic acids of the polynucleotide (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, the polynucleotide (e.g., mRNA) comprises uridine at one or more or all uridine positions of the nucleic acid. In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the polynucleotide (e.g., mRNA) may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C orA+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The polynucleotide (e.g., mRNA) may comprise one or more regions or parts that act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′ UTRs have features that play roles in translation initiation. They harbor signatures like Kozak sequences, which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTR also have been known to form secondary structures which are involved in elongation factor binding.

In some embodiments, the 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219, which are incorporated herein by reference in their entirety). CMV immediate-early 1 (IE1) gene (US2014/0206753, WO2013/185069, which are incorporated herein by reference in their entirety), the sequence GGGAUCCUACC (WO2014/144196) may also be used. In another embodiment, 5′ UTR of a TOP gene is a 5′ UTR of a TOP gene lacking the 5′ TOP motif (the oligopyrimidine tract) (e.g., WO/2015/101414, WO2015/101415, WO/2015/062738, WO2015/024667, WO2015/024667; 5′ UTR element derived from ribosomal protein Large 32 (L32) gene (WO/2015/101414, WO2015/101415, WO/2015/062738), 5′ UTR element derived from the 5′ UTR of an hydroxysteroid (17-b) dehydrogenase 4 gene (HSD17B4) (WO2015/024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

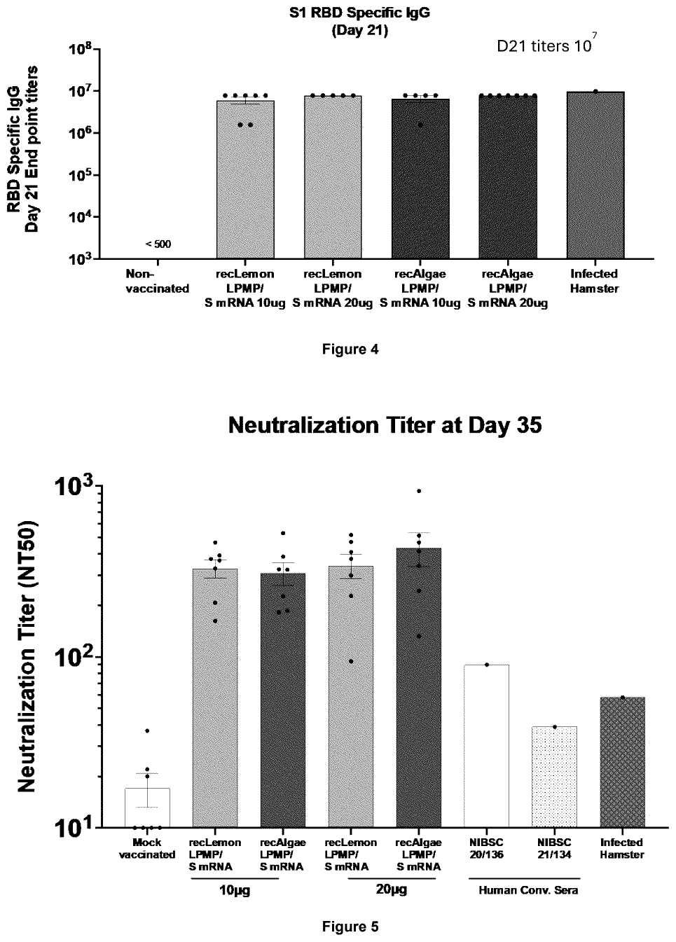

Introduction, removal or modification of 3′ UTR AU rich elements (AREs) can be used to modulate the stability of the polynucleotide (e.g., mRNA). When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection. [0371]3′ UTRs may be heterologous or synthetic. With respect to 3′ UTRs, globin UTRs, including Xenopus b-globin UTRs and human b-globin UTRs are known in the art (U.S. Pat. Nos. 8,278,063, 9,012,219, US2011/0086907). A modified b-globin construct with enhanced stability in some cell types by cloning two sequential human b-globin 3′UTRs head to tail has been developed and is well known in the art (US2012/0195936, WO2014/071963). In addition, a2-globin, al-globin, UTRs and mutants thereof are also known in the art (WO2015/101415, WO2015/024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US2014/0206753, WO2014152774), rabbit b globin and hepatitis B virus (HBV), a-globin 3′ UTR and Viral VEEV 3′ UTR sequences are also known in the art. In some embodiments, the sequence UUUGAAUU (WO2014/144196) is used. In some embodiments, 3′ UTRs of human and mouse ribosomal protein are used. Other examples include rps93′UTR (WO2015/101414), FIG. 4 (WO2015/101415), and human albumin 7 (WO2015/101415).

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 2010/0293625 and PCT/US2014/069155, which are herein incorporated by reference in their entirety. It should be understood that any UTR from any gene may be incorporated into the regions of a nucleic acid. Furthermore, multiple wild-type UTRs of any known gene may be utilized. It is also within the scope of the present disclosure to provide artificial UTRs that are not variants of wild type regions. These UTRs or portions thereof may be placed in the same orientation as in the transcript from which they were selected or may be altered in orientation or location. Hence, a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 2010/0129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins that are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest that share at least one function, structure, feature, localization, origin, or expression pattern.

The untranslated region may also include translation enhancer elements (TEE). As a non-limiting example, the TEE may include those described in US Application No. 2009/0226470, herein incorporated by reference in its entirety, and those known in the art. In vitro Transcription of RNA cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA, which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000,1000 to 3000, 1500 to 3000, or2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the polynucleotide (e.g., mRNA) comprises 5′ terminal cap, for example, 7mG(5′)ppp(5′)NlmpNp.

### Chemical Synthesis

Solid-phase chemical synthesis. The polynucleotide (e.g., mRNA) may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of the polynucleotide (e.g., mRNA) by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase-catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC).

The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the polynucleotide (e.g., mRNA) may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheo alveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Multivalent Vaccines

The LPMP/nucleic acid vaccines, as provided herein, may include RNA or multiple RNAs encoding two or more antigens of the same or different species. In some embodiments, the LPMP/nucleic acid vaccine includes an RNA or multiple RNAs encoding two or more coronavirus antigens.

In some embodiments, the RNA may encode 1, 2, 3, 4, 5, 6, 7, 8, 9, 10, 11, 12, or more coronavirus antigens.

In some embodiments, two or more different RNA (e.g., mRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The LPMP/nucleic acid vaccines, as provided herein, may include an RNA or multiple RNAs encoding two or more antigens of the same or different viral strains. Also provided herein are combination vaccines that include RNA encoding one or more coronavirus and one or more antigen(s) of a different organism. Thus, the vaccines of the present disclosure may be combination vaccines that target one or more antigens of the same strain/species, or one or more antigens of different strains/species, e.g., antigens which induce immunity to organisms which are found in the same geographic areas where the risk of coronavirus infection is high or organisms to which an individual is likely to be exposed to when exposed to a coronavirus.

### Vaccine Administration

In some embodiments, the LPMP/nucleic acid vaccines can be administered to a subject (e.g., a mammalian subject, such as a human subject), and the RNA polynucleotides are translated in vivo to produce an antigenic polypeptide (antigen). An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of the LPMP/nucleic acid vaccine provides an induced or boosted immune response as a function of antigen production in the cells of the subject. In some embodiments, an effective amount of the composition containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the LPMP/nucleic acid vaccines may be used for treatment or prevention of a coronavirus infection. The LPMP/nucleic acid vaccines may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

The LPMP/nucleic acid vaccines may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, the LPMP/nucleic acid vaccines may be administered intramuscularly, intranasally or intradermally, similarly to the administration of inactivated vaccines known in the art.

The LPMP/nucleic acid vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

### Kits

The present invention also provides a kit including a container having a PMP composition described herein. The kit may further include instructional material for applying or delivering the PMP composition to a plant in accordance with a method of the present invention. The skilled artisan will appreciate that the instructions for applying the PMP composition in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD).

### Examples

The invention now being generally described, it will be more readily understood by reference to the following examples, which are included merely for purposes of illustration of certain aspects and embodiments of the present invention, and are not intended to limit the invention.

### Example 1: Preparation and Modification of LPMP, and Formulation LPMP with mRNAs

The preparation of plant messenger packs (PMP), modification of PMP to prepare lipid reconstructed plant messenger packs (LPMP), and formulation of PMP and LPMP with mRNAs may be accomplished utilizing the methods disclosed in International Patent Application Publication No. WO 2021/041301, which is incorporated herein by reference in its entirety.

In particular, all the experimental protocols disclosed in Examples 1-17 of International Patent Application Publication No. WO 2021/041301, are incorporated herein by reference in their entirety, including: Example 1. Isolation of Plant Messenger Packs from plants; Example 2. Production of purified Plant Messenger Packs (PMPs); Example 3. Plant Messenger Pack characterization; Example 4. Characterization of Plant Messenger Pack stability; Example 5. Loading PMPs with cargo; Example 6. Increasing PMP cellular uptake by formulation of PMPs with ionic liquids; Example 7. Modification of PMPs using ionizable lipids; Example 8. Formulation of LPMPs with microfluidics; Example 9. mRNA loading and delivery into lipid-reconstructed PMPs using ionizable lipids; Example 10. Cellular uptake of natural and reconstructed PMPs, with and without ionizable lipid modifications; Example 11. Increasing PMP cellular uptake by formulation of PMPs with cationic lipids; Example 12. Modification of PMPs using cationic lipids; Example 13. mRNA loading and delivery into lipid-reconstructed PMPs using cationic lipids; Example 14. Cellular uptake of natural and reconstructed PMPs, with and without cationic lipid modifications; Example 15. Improved loading using the cationic lipids GL67 and Ethyl PC; Example 16. Optimization of lipid ratios for mRNA loading; and Example 17. Optimization of lipid ratios for plasmid loading.

### Example 2: Preparation of Vaccine

### Manufacture and Characterization of Polynucleotides

The manufacture of polynucleotides and/or parts or regions thereof may be accomplished utilizing the methods taught in International Patent Application Publication No. WO 2014/152027, which is incorporated herein by reference in its entirety. Purification methods may include those taught in International Patent Application Publication Nos. WO2014/152030 and WO2014/152031, which are incorporated herein by reference in their entirety. Detection and characterization methods of the polynucleotides may be performed as taught in International Patent Application Publication No. WO2014/144039, which is incorporated herein by reference in its entirety. Characterization of the polynucleotides may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Patent Application Publication Nos. WO2014/144711 and WO2014/144767, which are incorporated herein by reference in their entirety.

Additional details of the mRNA design and modifications may be found in WO 2021/154763 and WO 2021/188969, which are incorporated herein by reference in their entirety.

SARS-CoV-2 virus production

SARS-CoV-2 strain “Slovakia/SK-BMC5/2020”, originally provided by the European Virus Archive global (EVAg) (GISAID EPI_ISL_417879, https://www.european-virus-archive.com/virus/sars-cov-2-strain-slovakiask-bmc52020), produced and titered on Vero E6/TMPRSS2 cells, was used for hamster infection. The strain belonged to the GH clade.

Virus production was performed in T175 flasks seeded with 50×10⁶ Vero E6/TMPRSS2 cells and in a 40 mL final volume. Cell counts and viability were assessed by 0.25% trypan blue exclusion assay by ViCell apparatus. After 48 hours of infection time frame (with 0.001-0.005 MOI of SARS-CoV-2 virus), cytopathogenic effects were confirmed under microscope observation. Culture supernatant was harvested, centrifuged (5 min at 5000 g) and aliquoted (1 mL aliquots).

Virus stock TCID50 titers were determined on Vero E6/TMPRSS2 cells. About two hours before testing, cells were plated in 96-well plate at the density of 2×10⁴ cells per well in a volume of 200 μL of complete growth medium (DMEM 10% FCS). Cells were infected with serial dilutions of virus stock (8-plicates; 1st dilution 1:100; 5-fold serial dilutions) for 1 hour at 37° C. Fresh medium was added for 72 hours and a MTS/PMS assay is then performed, according to provider protocol (Promega, reference #G5430). Plates were read using an ELISA Plate reader and data recorded. Infectivity was expressed as TCID50/m572h based on the Spearman-Karber formula.

Sars-CoV-2 Spike (S) mRNA Sequence

The study was designed to test the immunogenicity in hamster and/or mice of the candidate coronavirus vaccines comprising an mRNA of Table 1 encoding a coronavirus antigen (e.g., the spike (S) protein), such as a SARS-CoV-2 antigen.

TABLE 1
SARS-CoV-2 spike (S) mRNA sequence (with modified cap)

| Element | Description | Position |
|---|---|---|
| cap | A modified 5′-cap1 structure (m⁷G⁺m^{3′}-5′-ppp-5′-Am) | 1-2 |
| 5′-UTR | 5′-untranslated region derived from human alpha-globin RNA with an | 3-54 |
| | optimized Kozak sequence | |
| sig | S glycoprotein signal peptide (extended leader sequence), which | 55-102 |
| | guides translocation of the nascent polypeptide chain into the | |
| | endoplasmic reticulum. | |
| S protein_mut | Codon-optimized sequence encoding full-length SARS-CoV-2 spike | 103-3879 |
| | (S) glycoprotein containing mutations K986P and V987P to ensure | |
| | the S glycoprotein remains in an antigenically optimal pre-fusion | |
| | conformation; stop codons: 3874-3879 (underlined) | |
| 3′-UTR | The 3′ untranslated region comprises two sequence elements derived | 3880-4174 |
| | from the amino-terminal enhancer of split (AES) mRNA and the | |
| | mitochondrial encoded 12S ribosomal RNA to confer RNA stability | |
| | and high total protein expression. | |
| poly(A) | A 110-nucleotide poly(A)-tail consisting of a stretch of 30 adenosine | 4175-4284 |
| | residues, followed by a 10-nucleotide linker sequence and another 70 | |
| | adenosine residues. | |

Formulation of LPMP with SARS-CoV-2 S mRNA

### General Protocols and Experimental Designs

The protocols and detailed experimental procedures of the preparation of plant messenger packs (PMP), and modification of PMP to prepare lipid reconstructed plant messenger packs (LPMP), and formulations of PMP and LPMP with mRNAs have been discussed in Example 1, which lists all the experimental protocols disclosed in Examples 1-17 of International Patent Application Publication No. WO 2021/041301, all of which are incorporated herein by reference in their entirety. These protocols and detailed experimental procedures were followed when preparing PMP, LPMP, and PMP or LPMP formulated with mRNAs in this example.

Briefly, the isolation and purification of crude plant messenger packs (PMPs) from lemon and algae, and characterization of these PMPs followed the experimental designs and protocols for plants sources described in Example 1. Isolation of Plant Messenger Packs from plants; Example 2.

Production of purified Plant Messenger Packs (PMPs); Example 3. Plant Messenger Pack characterization; and Example 4. Characterization of Plant Messenger Pack stability, all of International Patent Application Publication No. WO 2021/041301, which is incorporated herein by reference in its entirety.

The modifications of natural PMP or reconstructed lemon or algae LPMPs with cholesterol and PEG-lipid followed the experimental designs and protocols for plants sources described in Example 6. Increasing PMP cellular uptake by formulation of PMPs with ionic liquids; Example 7. Modification of PMPs using ionizable lipids; Example 10. Cellular uptake of natural and reconstructed PMPs, with and without ionizable lipid modifications; Example 11. Increasing PMP cellular uptake by formulation of PMPs with cationic lipids; Example 12. Modification of PMPs using cationic lipids; and Example 14. Cellular uptake of natural and reconstructed PMPs, with and without cationic lipid modifications, all of International Patent Application Publication No. WO 2021/041301, which is incorporated herein by reference in its entirety.

Formulations of the PMPs and lemon-lipid- or algae-lipid-reconstructed LPMPs with mRNAs followed the experimental designs and protocols for nucleic acids loading described in Example 5. Loading PMPs with cargo; Example 8. Formulation of LPMPs with microfluidics; Example 9. mRNA loading and delivery into lipid-reconstructed PMPs using ionizable lipids; Example 13. mRNA loading and delivery into lipid-reconstructed PMPs using cationic lipids; Example 15. Improved loading using the cationic lipids GL67 and Ethyl PC; Example 16. Optimization of lipid ratios for mRNA loading; and Example 17. Optimization of lipid ratios for plasmid loading, all of International Patent Application Publication No. WO 2021/041301, which is incorporated herein by reference in its entirety.

### The Formulations for LPMP/mRNA Vaccine

This example describes the formulation of lemon lipid and algae lipid reconstructed LPMP formulated with ionizable lipids, sterols, and PEG lipids, to encapsulate mRNA (e.g., SARS-CoV-2 spike (S) mRNA sequence) for LPMP/mRNA vaccine formulation. The LPMP/mRNA vaccine with lemon lipid reconstructed LPMP is typically referred to as A and the LPMP/mRNA vaccine with algae lipid reconstructed LPMP is typically referred to as B, e.g., as shown in Table 2 below.

In this example, lemon and algae PMP lipids were used as the PMP natural lipids; C12-200 [1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol)] was used as the ionizable lipids; cholesterol (14:0) was used as the sterols; DMPE-PEG2k was used as model PEGylated lipids, and SARS-CoV-2 spike (S) mRNA sequence (described herein) was used as the loaded mRNA.

LNP formulations. A LNP (lipid nanoparticle) formulation, as control, was prepared to result in ionizable lipid:structural lipid:sterol:PEG-lipid (C12-200:DOPE:cholesterol (14:0): DMPE-PEG2k) at a molar ratio of 35:16:46.5:2.5, respectively. To prepare this formulation, the above lipids were solubilized in ethanol, mixed at the above molar ratios, and diluted in ethanol (organic phase) to obtain total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 15:1.

LPMP formulations. Reconstructed lemon (recLemon) LPMP formulation was prepared to result in ionizable lipid:natural lipids:sterol:PEG-lipid (C12-200:lemon lipid:cholesterol (14:0): DMPE-PEG2k) at a molar ratio of 35:50:12.5:2.5, respectively. To prepare this formulation, the above lipids were solubilized in ethanol, except for lemon lipid, which was solubilized in 4:1 DMF:methanol. The lipids were then mixed at the above molar ratios, and diluted to obtain total lipid concentration of 5.5 mM. The mRNA solution (aqueous phase) was prepared with RNAse-free water and 100 mM citrate buffer pH 3 for a final concentration of 50 mM citrate buffer.

Reconstructed algae (recAlgae) LPMP formulation was prepared to result in ionizable lipid:natural lipids:sterol:PEG-lipid (C12-200:algae lipid:cholesterol (14:0): DMPE-PEG2k) at a molar ratio of 35:20:42.5:2.5, respectively. To prepare this formulation, the above lipids were solubilized in ethanol, except for algae lipid, which was solubilized in 4:1 DMF:methanol. The lipids were then mixed at the above molar ratios, and diluted to obtain total lipid concentration of 5.5 mM. The mRNA solution (aqueous phase) was prepared with RNAse-free water and 100 mM citrate buffer pH 3 for a final concentration of 50 mM citrate buffer.

The lipid mixture and mRNA solution were mixed at a 1:3 ratio by volume, respectively, on the NanoAssemblr Ignite (Precision Nanosystems) at a total flow rate of 9 mL/min. The resulting formulations were then loaded into Slide-A-Lyzer G2 dialysis cassettes (10 k MWCO) and dialyzed in 200 times sample volume of 1×PBS for 4 hours at room temperature with gentle stirring. The PBS solution was refreshed, and the formulations were further dialyzed for at least 14 hours at 4° C. with gentle stirring. The dialyzed formulations were then collected and concentrated by centrifugation at 3000×g (Amicon Ultra centrifugation filters, 100 k MWCO).

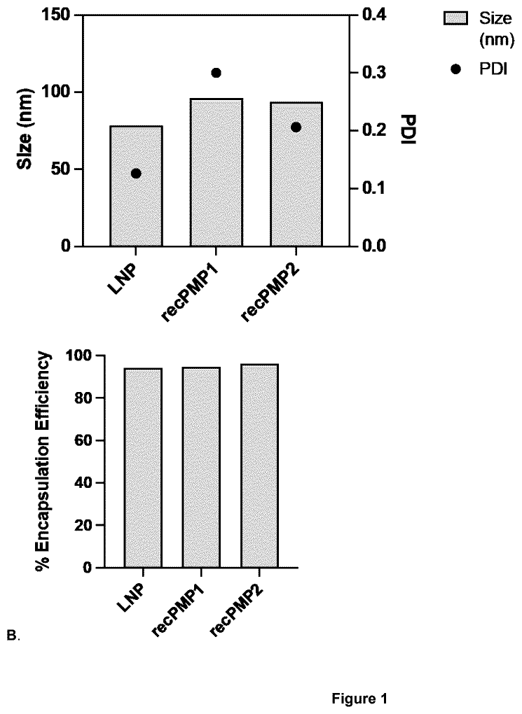

The concentrated particles were characterized for size, polydispersity, and particle concentration using Zetasizer Ultra (Malvern Panalytical). The results are shown in FIG. 1A. The mRNA encapsulation efficiency was characterized by Quant-iT RiboGreen RNA Assay Kit (ThermoFisher Scientific). The results are shown in FIG. 1B. The particles were diluted to the desired mRNA concentration to get a final 10% sucrose solution in PBS. The formulations were then flash frozen in liquid nitrogen.

The resulting formulations with mRNA are shown in Table 2.

TABLE 2
The formulations with mRNA

| | Ionizable:Structural:Sterol:PEG- | | | | PEG- | mRNA | | Size | | Encapsulation |
|---|---|---|---|---|---|---|---|---|---|---|
| Formulation | lipid Molar ratio | Ionizable | Structural | Sterol | lipid | Cargo | N:P | (nm) | PDI | efficiency (%) |
| LNP | 35:16:46.5:2.5 | C12-200 | DOPE | Chol | DMPE- | SARS- | 15:1 | 78.47 | 0.126 | 94.4 |
| | | | | | PEG2k | CoV-2 | | | | |
| | | | | | | mRNA | | | | |
| LPMP/SARS- | 35:50:12.5:2.5 | C12-200 | Naturally | Chol | DMPE- | SARS- | | 96.49 | 0.300 | 94.8 |
| CoV-2 A | | | derived | | PEG2k | CoV-2 | | | | |
| (recPMP1) | | | lemon lipid | | | | | | | |
| LPMP/SARS- | 35:20:42.5:2.5 | C12-200 | Naturally | Chol | DMPE- | SARS- | | 93.96 | 0.206 | 96.3 |
| CoV-2 B | | | derived | | PEG2k | CoV-2 | | | | |
| (recPMP2) | | | algae lipid | | | | | | | |

### Example 3: Vaccination and Coronavirus Challenge Design

The LPMP/SARS-CoV-2 vaccine formulation was obtained according to Example 2.

The exemplified tested samples were reconstructed lemon (recLemon) LPMP formulation with SARS-CoV-2 (LPMP/SARS-CoV-2 A) and reconstructed algae (recAlgae) LPMP formulation with SARS-CoV-2 (LPMP/SARS-CoV-2 B).

The design of single intramuscular vaccination of the LPMP/SARS-CoV-2 vaccine and SARS-CoV2 challenge of hamsters administered with the LPMP/SARS-CoV-2 vaccine is shown in Scheme 1.

### Treatment Schedule

As showing in Scheme 1, healthy Golden Syrian Hamsters (females), 6-8 weeks old at reception, were obtained. The animals were weighed and then homogenously allocated into groups as shown in Table 3 below.

The test samples (control vector & vaccine vector) were provided as a frozen solution and stored at −20° C. Inoculations were performed after thawing overnight at −4° C. prior to dosing. The test samples (control vector & vaccine vector) were injected using the intra-muscular (IM) route, in one upper thigh, according to the treatment schedule in Table 3.

TABLE 3
Treatment schedule

| | No. | | Treatment | Dosage/ | Total | Virus | End |
|---|---|---|---|---|---|---|---|
| Group | animals | Test | (IM) | injection | quantity**** | (IN, d 28) | point |
| A | 7 | recLemon LPMP + | 1x dose on | 50 μL | 0.46 mL | 10⁵ pfu | d 32 |
| (n = 14) | | mRNA-EPO* | d 0 | | | | (4 dpi) |
| | 7 | recLemon LPMP + | 1x dose on | 50 μL | 0.46 mL | 10⁵ pfu | d 35 |
| | | mRNA-EPO | d 0 | | | | (7 dpi) |
| B | 7 | recLemon LPMP + | 1x dose on | 50 μL | 0.46 mL | 10⁵ pfu | d 32 |
| (n = 14) | | mRNA-S** (1x dose) | d 0 | | | | (4 dpi) |
| | 7 | recLemon LPMP + | 1x dose on | 50 μL | 0.46 mL | 10⁵ pfu | d 35 |
| | | mRNA-S (1x dose) | d 0 | | | | (7 dpi) |
| C | 7 | recLemon LPMP + | 1x dose on | 50 μL | 0.46 mL | 10⁵ pfu | d 35 |
| (n = 7) | | mRNA-S (2x dose) | d 0 | | | | (7 dpi) |
| D | 7 | recAlgae LPMP + | 1x dose on | 50 μL | 0.46 mL | 10⁵ pfu | d 35 |
| (n = 7) | | mRNA-S*** (1x dose) | d 0 | | | | (7 dpi) |
| E | 7 | recAlgae LPMP + | 1x dose on | 50 μL | 0.46 mL | 10⁵ pfu | d 35 |
| (n = 7) | | mRNA-S (2x dose) | d 0 | | | | (7 dpi) |

*recLemon LPMP + mRNA-EPO: control vector (non-vaccinated)
**recLemon LPMP + mRNA-S: vaccine vector (recLemon LPMP/SARS-CoV-2 vaccine)
**recAlgae LPMP + mRNA-S: vaccine vector (recAlgae LPMP/SARS-CoV-2 vaccine)
****including a 30% test product excess (average hamster weight of 120 g).

### Blood Sampling Before SARS-CoV-2 Viral Challenge

On 4 time points (d0 t+6h/d1/d10/d21), blood was collected via puncture at the jugular vein on isoflurane-anesthetized animals. Around 300 μL of blood was transferred into collection tubes with clot activator and allowed to coagulate for 30 minutes. Tubes were then centrifuged (2000 g, 10 minutes, room temperature) to obtain around 150 μL serum.

On d0 (t+6h), d10 and d21: the blood sampling procedure was performed on certain groups of animals to be euthanized on d35 (7 dpi; n=42). See Table 3.

On d1: the blood sampling procedure was performed on certain groups of animals to be euthanized on d32 (4 dpi; n=21). See Table 3.

Serum samples were stored in propylene tubes at −80° C. until further use for evaluation of early cytokine & EPO expression (d0 t+6/d1) or antibody response by Multiplex ELISA & neutralization assay.

### SARS-CoV-2 Challenge (Day 28)

All the animals received SARS-CoV-2 on Day 28, administered by intranasal route (IN) under a total volume of 70 μL (35 μL per nostril) on isoflurane-anesthetized animals. An intranasal dose of 10⁵ pfu TCID₅₀ per animal was administered.

### Termination of Animals on Day 32 (4 Dpi)

As shown in Table 3, certain groups of animals (n=21) underwent terminal euthanasia on Day 32 (i.e., 4 days post-infection, dpi).

Nasal and lung (superior right lung lobe) tissues were collected by placing the tissues in RNA overnight at 4° C., then stored at −80° C. until RNA extraction for quantification of viral load & cytokine response profiling by qRT-PCR. Middle, post-caval and inferior right lung lobes were snap frozen in liquid nitrogen (one lobe per tube), then stored at −80° C. until quantification of viral infectious particles (TCID₅₀).

### Termination of Animals on Day 35 (7 Dpi)

The remaining animals (n=42) underwent terminal euthanasia on Day 35 (i.e., 7 dpi). Maximal terminal blood sampling was performed (for serum sample preparation) before lung and spleen collection.

Left lung was put in formalin for histology for at least 24 hours.

Spleens was further used in an ELISPOT IFNγ assay.

Serum samples were stored in propylene tubes at −80° C. until further use for evaluation of antibody response by Multiplex ELISA & neutralization assay.

### Elisa for Serum Cytokine & EPO Quantification

Cytokine and EPO levels in serum (ELISA) were quantified and monitored at 2 time points (d0 t+6 h and d1).

EPO was evaluated at time point (d0 t+6h), using U-PLEX Human EPO Assay Kit (MSD, ref. K151VXK).

IL-6 was evaluated at 2 time points (d0 t+6 h and d1), using Hamster Interleukin 6, IL-6 ELISA Kit (Cusabio, ref. CSB-E14304Ha).

Other cytokines (e.g., CXCL-12, SCF, IFNγ, TNFα, IL2, IL4, MCP1, and IL18) were evaluated at 2 time points (d0 t+6h and d1). The corresponding ELISA kits were used for the monitoring of these cytokine levels.

### Antibody Response Evaluation by Multiplex ELISA (Day 10/21/35)

The analysis of the antibody responses (IgG) against SARS-CoV-2 was performed using V-PLEX SARS-CoV-2 Panel 2 plates from Meso Scale Discovery (kit K15383U). Responses were quantified for hamster antibodies targeting the S, RBD and N antigens of SARS-CoV-2 using a multiplex approach.

Plates were provided with antigens on spots in the wells of a 96-well plate. Antibodies in the sample bind to the antigens on the spots and anti-hamster IgG antibodies conjugated with MSD SULFO-TAG were used for detection. The plate were read on a MESO Quickplex SQ120 imager which measures the light emitted from the MSD SULFO-TAG.

Spot 1: SARS-CoV-2 Spike=soluble ectodomain with T4 trimerization domain; C-terminal Strep-Tag and His-Tag.

Spot 3: SARS-CoV-2 Nucleocapsid=Full length Nucleocapsid; C-terminal His-Tag.

Spot 10: SARS-CoV-2 S1 RBD=R319-F541 of the Spike sequence; C-terminal His-Tag.

### Neutralizing Antibody Response Evaluation (Day 10/21/35)

Serum samples were analyzed using a live SARS-CoV-2 cytopathogenicity-based assay with Vero E6/TMPRSS2 cells, as follows:

Vero E6/TMPRSS2 cells were counted and their viability were assessed by 0.25% trypan blue exclusion assay by ViCell apparatus. About 16 hours before testing, cells were plated in 96-well plate at the density of 2×10⁴ cells per well in a volume of 200 μL of complete growth medium.

Heat-inactivated (56° C. for 30 min) sera containing neutralizing antibodies were first serially diluted (3-fold steps), then mixed (1:1 v:v) with 0.01 MOI per well of SARS-CoV-2 virus for 30 minutes at room temperature to allow antibodies in the sera to bind to the viruses. The first final serum dilution after mixture with virus was 1:5.

The virus-antibody mixture (50 μL) were added to Vero E6/TMPRSS2 cells (after removing the cell growth medium) and incubated for 2 hours at 37° C., 5% CO₂ to allow viruses to infect targeted cells.

Afterwards, 150 μL of complete cell growth medium (containing 2% FBS) was added to each well. The plates were then incubated for 48 hours at 37° C., 5% CO₂.

Plate revelation was performed using the CellTiter 96@AQueous Non-Radioactive Cell Proliferation Assay, performed according to protocol (Promega, reference #G5430), determining the number of viable cells in proliferation assays.

After removing 100 μL of supernatant, 100 μL of fresh medium and 20 μL of MTS/PMS reagent were added. Plates were read using an ELISA Plate reader.

Neutralizing antibody titer (NT50) is defined as the reciprocal of the highest serum dilution that provides >50% inhibition of virus infectivity.

### ELISPOT IFNγ Evaluation of Anti-SARS-CoV-2/S T Cell Responses (Day 35)

T cell responses against SARS-CoV-2 antigens were evaluated in a hamster ELISPOT IFNγ assay (Mabtech, reference #3102-2A).

T cell responses were assessed against SARS-CoV-2 Spike protein using specific 15mer peptide scan pools with 11 amino-acid overlap (JPT; PepMix SARS-COV2 Spike, reference PM-WCPV—S-2).

Splenocytes were isolated on a cell strainer and filtered on 70 μm filters before and after red blood cell lysis. Live splenocytes were counted and their viability was assessed by 0.25% trypan blue exclusion assay by ViCell apparatus.

ELISPOT assay was performed on triplicates with two cell density (200×10³ & 60×10³ cells per well), comparing different conditions: medium only; PMA (20 ng/mL) & ionomycin (1 μM) mix as positive control; peptide pool (2 μg/mL). For the PMA/ionomycin positive control, lower cell densities were used (60×103 & 20×10³ cells per well). [0466]24 hours after ELISPOT onset, the IFNγ producing cells were revealed. The plates were analyzed using an AID (#ELR08) ELIPOT plate reader.

### Clinical Monitoring—Animal Body Weight

Animal body weight was monitored once a week after vaccination, then daily after SARS-CoV-2 infection.

Virus Load Determination in Lungs & Nasal Tissues by Genomic qRT-PCR (Day 32) (4 Dpi)

Quantification of viral load by RT-qPCR was done from lung & nasal tissues using viral ORF1ab gene.

Extraction of viral RNA was performed using the Macherey Nagel NucleoSpin 96 RNA, 96-well kit for RNA purification (ref.740709.4). RNA was frozen at −80° C. until qRT-PCR.

RT was performed with the High Capacity cDNA Reverse Transcription Kit from Applied Biosystem (ref #4368813).

cDNA quantification by quantitative PCR was performed with primers conditions targeting ORF1ab gene. Amplifications were performed using a QuantStudio 7 Flex from Applied Biosystem and adjoining software.

Primers and Probes

| Name | Sequences (5′-3′) |
|---|---|
| ORF1ab gene/nCoV | |
| ORF1ab_Fw | CCGCAAGGTTCTTCTTCGTAAG |
| ORF1ab_Rv | TGCTATGTTTAGTGTTCCAGTTTTC |

### Virus TCID₅₀ Determination in Lungs (Day 32) (4 Dpi)

The tissue culture infective dose that causes 50% cytotoxicity (TCID₅₀) assay is a quantitative method for assessing the infectivity of a virus stock. One TCID₅₀ is defined as the amount of pathogen that causes death of 50% of cells (Reed and Muench, 1938), so TCID₅₀ depends on the ability of the virus to kill the cells in culture. Infectivity was expressed as TCID₅₀/mL/48 hour based on the Spearman-Karber formula.

Vero E6/TMPRSS2 cells were counted and their viability were assessed by 0.25% trypan blue exclusion assay by ViCell apparatus.

One day before testing, cells were plated in 96-well plate at the density of 2×10⁴ cells per well in a volume of 200 μL of complete growth medium (DMEM 10% FCS).

Cells were infected with serial dilutions of the lung homogenate (triplicate) for 1 hour at 37° C.

Fresh medium were added for 48 hours. [0476]48 hours after cell infection, a MTS/PMS assay was performed, according to the protocol (Promega ref #G5430). After discarding all supernatant, 100 μL of fresh medium and 20 μL of MTS/PMS reagent were added to the culture wells. After a maximum of 4 hours, plates were read using an Elisa Plate reader and data were recorded (OD value in negative cell control >1.500).

### Example 4: Vaccination and Coronavirus Challenge of Hamsters

Two vaccine formulations were prepared according to Example 2: LPMP/SARS-CoV-2 A (reconstructed lemon (recLemon) LPMP formulation with SARS-CoV-2) and LPMP/SARS-CoV-2 B (reconstructed algae (recAlgae) LPMP formulation with SARS-CoV-2).

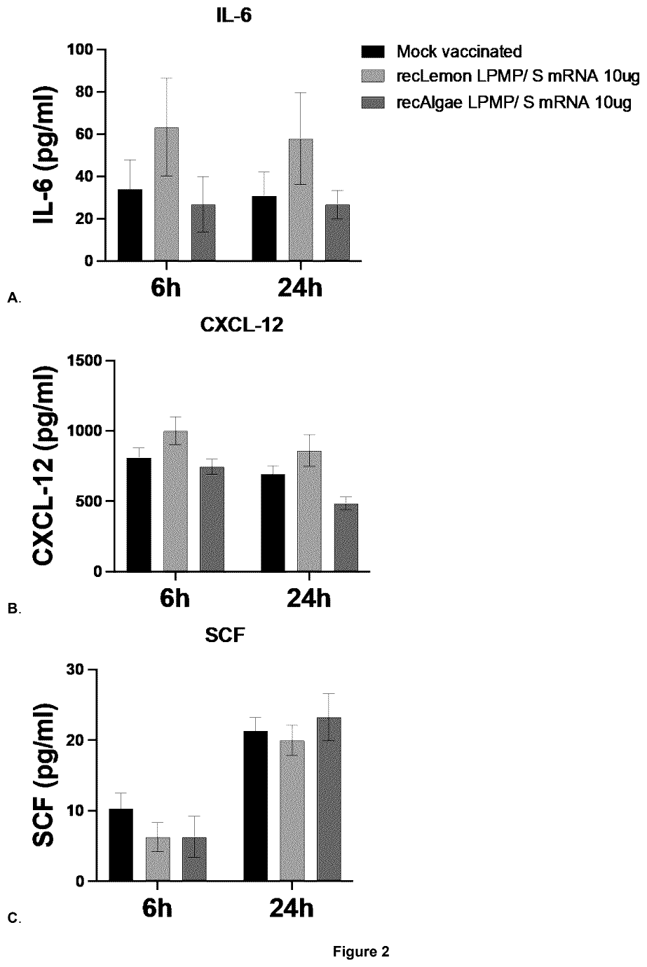

The protocols of vaccination and coronavirus challenge were those described in Example 3. The administration of these LPMP/SARS-CoV-2 formulations was based on treatment schedule shown in Table 3, and the results of the vaccination and SARS-CoV-2 challenge of these hamsters are shown in FIGS. 2-10.

### Serum Cytokines

The levels of cytokine IL6 (FIG. 2A), chemokine CXCL12 (FIG. 2B), and cytokine SCF (stem cell factor) (FIG. 2C) in the serum of the hamsters at 6 and 24 hours, after a single dose intramuscular vaccination of LPMP/SARS-CoV-2 vaccines in the hamsters were evaluated and monitored. The results are shown in FIG. 2, which indicates that single dose intramuscular vaccination of the LPMP/SARS-CoV-2 vaccine induced pro-inflammatory cytokine and chemokine 6 and 24 hours after vaccination in the hamsters. No detectable level of IFNγ, IL2, IL4, MCP1, TNFα, and IL18 were found in hamster plasma at 6 and 24 hours post dosing. However, both LPMP/SARS-CoV-2 A (10 μg) and LPMP/SARS-CoV-2 B (10 μg) induced some level of pro-inflammatory cytokine IL6 (FIG. 2A) and chemokine CXCL12 (FIG. 2B) at 6 and 24 hours post dosing. Both LPMP/SARS-CoV-2 A (10 μg) and LPMP/SARS-CoV-2 B (10 μg) also induced some level of cytokine SCF (stem cell factor) (FIG. 2C) at 6 and 24 hours post dosing.

### Antibody and Neutralizing Antibody Responses

The antibody responses (IgG) targeting the S and S1 RBD antigens of SARS-CoV-2 in the serum of the hamsters at D10, D21, and D35, after intramuscular vaccination of LPMP/SARS-CoV-2 vaccines in the hamsters, were evaluated.

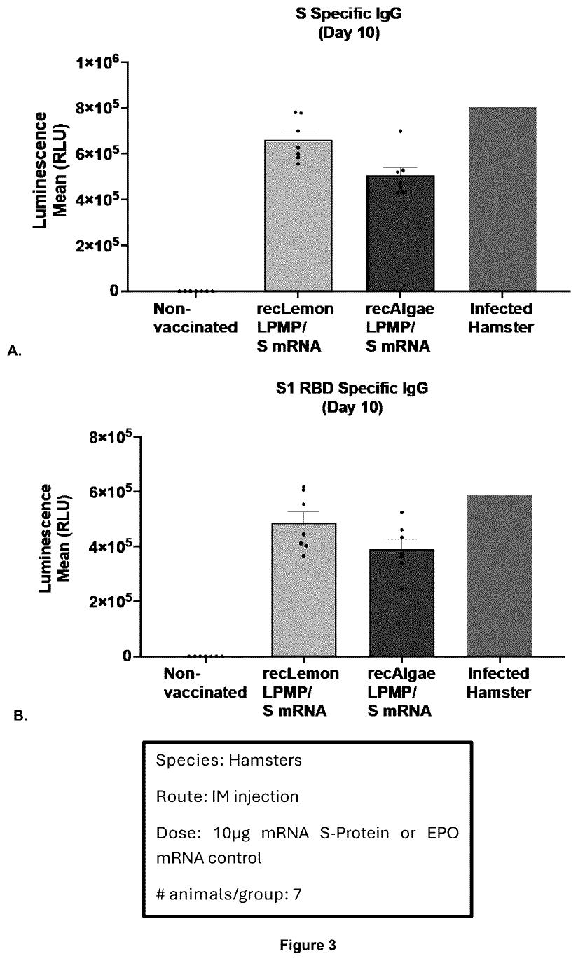

FIG. 3A-3B show that a single dose intramuscular vaccination of the LPMP/SARS-CoV-2 vaccine induced high level of S specific and S1 RBD specific IgG 10 days after vaccination in the hamsters. The detected high levels of SARS-CoV-2 specific antibody induced by the LPMP/SARS-CoV-2 vaccine were very close to what was observed in the SARS-CoV-2 infected hamsters. Among the two vaccine formulations, LPMP/SARS-CoV-2 A (lemon-derived LPMP) vaccine induced a higher level of SARS-CoV-2 specific antibody 10 days after vaccination in the hamsters.

FIG. 4 shows that the intramuscular vaccination of the LPMP/SARS-CoV-2 vaccine induced a very high level of S1 RBD specific IgG 21 days after vaccination in the hamsters. The detected high levels of SARS-CoV-2 specific antibody induced by the LPMP/SARS-CoV-2 vaccine were very close to what was observed in the SARS-CoV-2 infected hamsters. Also, the LPMP/SARS-CoV-2 vaccine induced very high titers of RBD specific antibodies (D21 titers: 10⁷), even with a relatively low LPMP/SARS-CoV-2 vaccine dosage (LPMP/SARS-CoV-2 B, 20 μg).

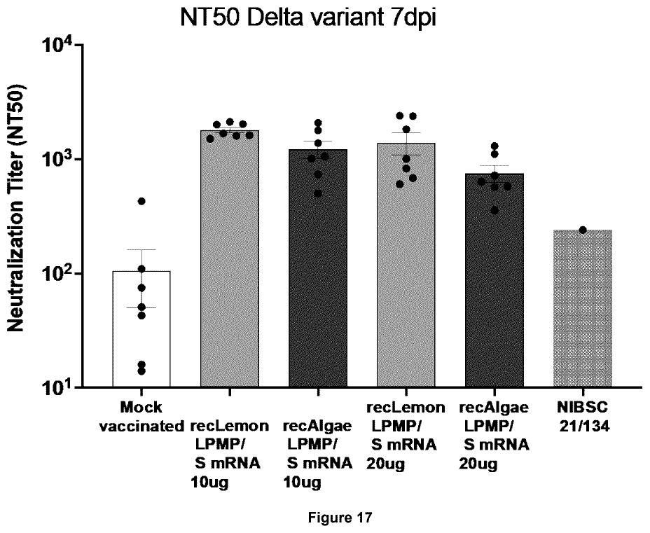

FIGS. 5 and 17 show that the intramuscular vaccination of the LPMP/SARS-CoV-2 vaccine induced a robust neutralizing antibody titer in the serum of the hamsters at 35 days, after intramuscular vaccination of LPMP/SARS-CoV-2 vaccines, which was effective for multiple viral variants, in the hamsters.

### Anti-SARS-CoV-2/S T Cell Responses

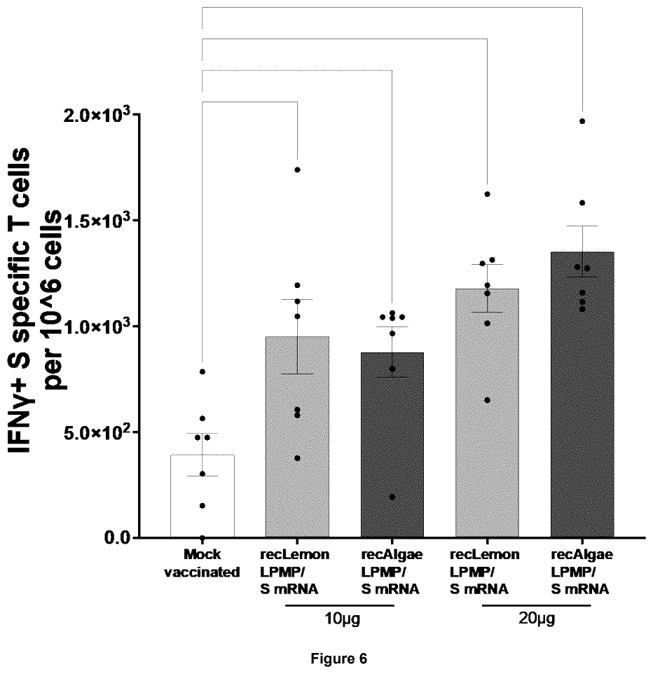

FIG. 6 shows that the intramuscular vaccination of the LPMP/SARS-CoV-2 vaccine induced SARS-CoV2 Spike protein specific T cells.

Vaccination of the Hamsters with the LPMP/SARS-CoV-2 Vaccine Protected the Hamsters from SARS-CoV-2 Challenge

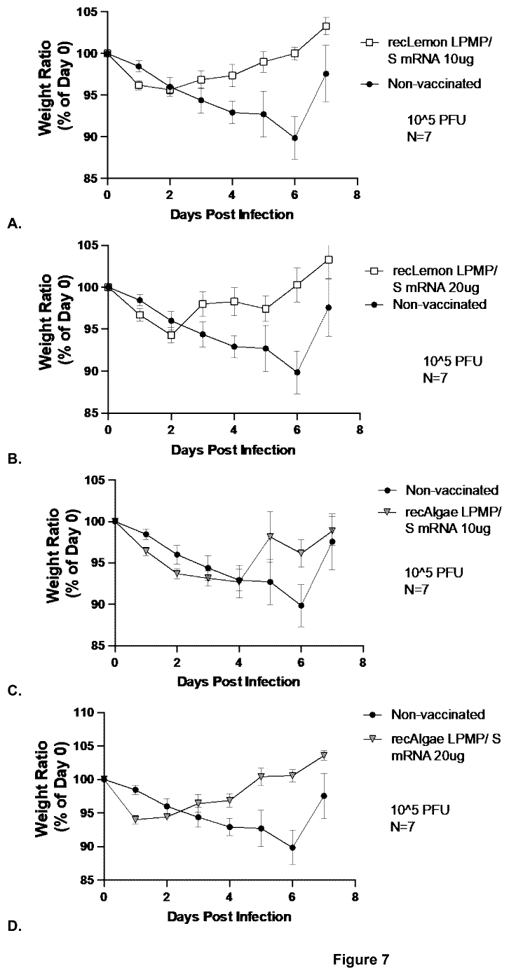

The ability of the LPMP/SARS-CoV-2 vaccine to protect the hamsters from the SARS-CoV-2 challenge are shown in FIGS. 7-10.

FIGS. 7A-7D show that the LPMP/SARS-CoV-2 vaccinated hamsters mounted a robust vaccine-induced immune response and were protected from the SARS-CoV2 challenge as measured by loss of body weight. For both the LPMP/SARS-CoV-2 A and LPMP/SARS-CoV-2 B formulations, a single dosage (10 μg) intramuscular vaccination protected the hamsters from the SARS-CoV-2 challenge.

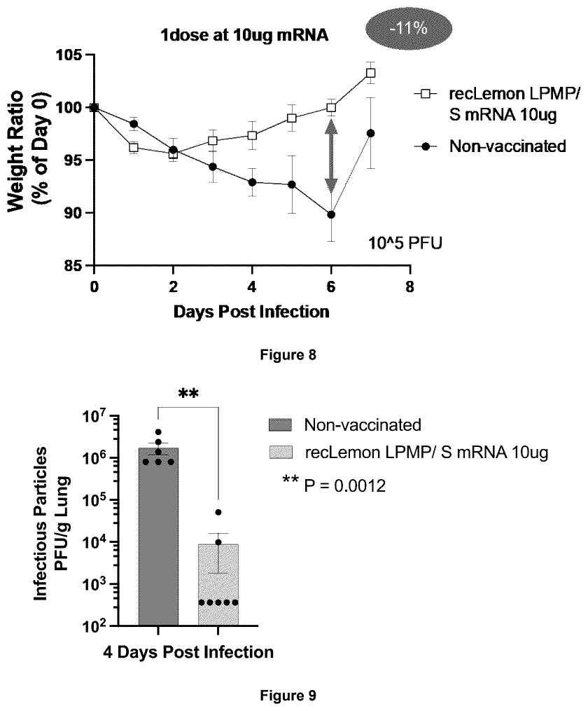

As shown in FIG. 8, a single dosage (10 μg) intramuscular vaccination of the LPMP/SARS-CoV-2 A vaccine formulation mounted a robust vaccine-induced immune response in the hamsters and achieved protection in the hamsters from the SARS-CoV-2 challenge similar to best benchmark (11% loss of body weight).

As shown in FIG. 9, a single dosage (10 μg) intramuscular vaccination of the LPMP/SARS-CoV-2 A vaccine formulation mounted a robust vaccine-induced immune response in the hamsters 4 days post infection, and protected the hamsters from the SARS-CoV-2 challenge by significantly decreasing the infectious viral particles (determined by virus TCID₅₀) in the lungs.

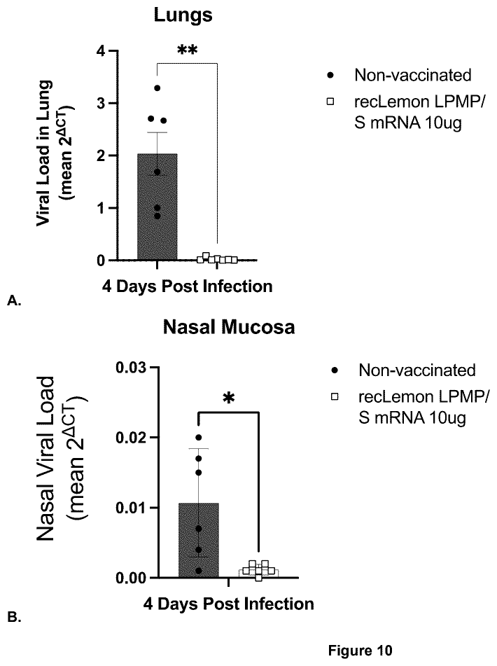

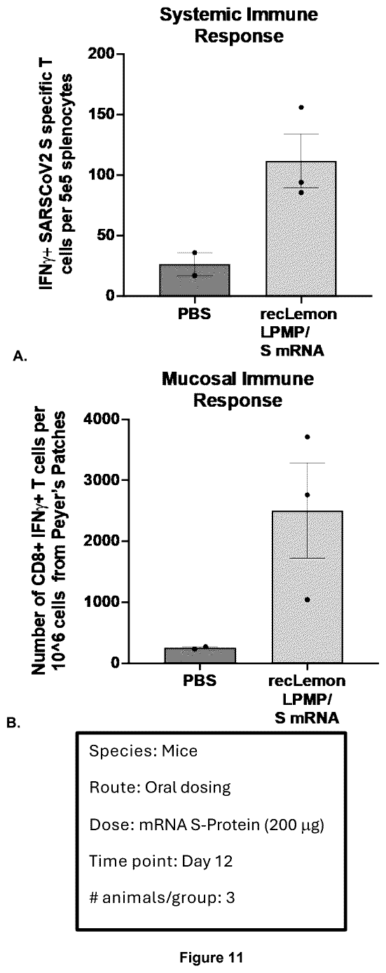

As shown in FIGS. 10A-10B, a single dosage (10 μg) intramuscular vaccination of the LPMP/SARS-CoV-2 A vaccine formulation mounted a robust vaccine-induced immune response in the hamsters 4 days post infection, and protected the hamsters from the SARS-CoV-2 challenge by significantly decreasing the viral load in the lungs (FIG. 10A) and nasal mucosa (FIG. 10B).

Collectively, FIGS. 8-10 indicate that a single dosage (10 μg) intramuscular vaccination of the LPMP/SARS-CoV-2 A vaccine formulation effectively protected the hamsters from the SARS-CoV2 challenge as measured by loss of body weight, infectious viral particles in the lungs, and viral load in both the lungs and nasal mucosa.

### Example 5: Vaccination and Coronavirus Challenge of Mice

If not specificized, the protocols of vaccination and coronavirus challenge and characterization of immunization responses were similar to those described in Example 3, except the animals used were mice in this example.

Two vaccine formulations were prepared according to Example 2: LPMP/SARS-CoV-2 A and LPMP/SARS-CoV-2 B. The control was PBS.

### Oral Dosage

Three mice were orally administered with a single dosage of LPMP/SARS-CoV-2 A (containing S mRNA 200 μg). Anti-SARS-CoV-2/S T cell responses were evaluated in an ELISPOT IFNγ assay at D12. Antibody responses (IgM, IgG, IgA) were evaluation by Multiplex ELISA at D12, and D21, respectively.

FIGS. 11A-11B show the results of T cell responses against S antigen of SARS-CoV-2 in the spleens of the mice (FIG. 11A) and in the Peyer's patches of the mice (FIG. 11B) at 12 days, after oral vaccination of LPMP/SARS-CoV-2 A (containing S mRNA 200 μg) in the mice. The results indicate that a single dosage oral vaccination of the LPMP/SARS-CoV-2 A vaccine formulation induced systemic and mucosal SARS-CoV-2 immune responses in mouse. A single dosage oral vaccination of the LPMP/SARS-CoV-2 A vaccine formulation also induced mucosal and cytotoxic CD4 and CD8 T cells in Peyer's patches.

Moreover, the oral vaccination of the LPMP/SARS-CoV-2 vaccine also induced a high level of S specific IgM at 12 days after vaccination in the mice, and a low level of antigen specific IgG at 21 days after vaccination in the mice. Similar oral vaccination studies also showed a low level of IgA in the gut and lungs of the mice.

### Intranasal Dosage

Three mice were intranasally administered with a single dosage of LPMP/SARS-CoV-2 A vaccination (containing S mRNA 0.1 μg, 10 μg). Anti-SARS-CoV-2/S T cell responses were evaluated in an ELISPOT IFNγ assay at D12. Cytokine levels (e.g., TNFα) in blood (ELISA) were quantified at D12.

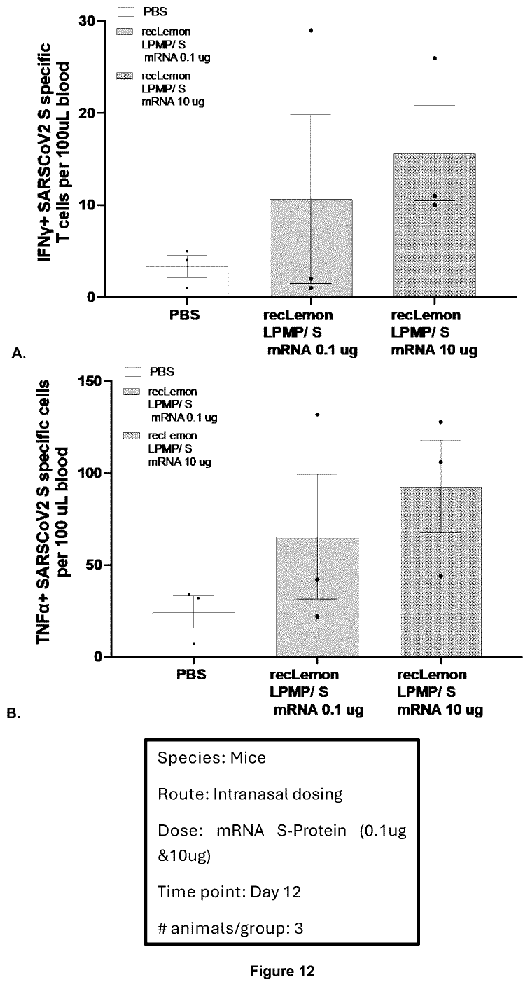

FIG. 12A shows the results of T cell responses against S antigen of SARS-CoV-2 in the mice at 12 days, after intranasal vaccination of LPMP/SARS-CoV-2 A (containing S mRNA 0.1 μg, 10 μg) in the mice. FIG. 12B shows the levels of cytokine TNFα in the blood of the mice at 12 days, after intranasal vaccination of LPMP/SARS-CoV-2 A (containing S mRNA 0.1 μg, 10 μg) in the mice.

The results indicate that a single dosage intranasal vaccination of the LPMP/SARS-CoV-2 A vaccine formulation induced systemic SARS-CoV2 Spike specific T cells 12 days post immunization.

### Intramuscular Dosage

Mice were intramuscularly administered either a single dose or two doses of LPMP/SARS-CoV-2 A vaccination (containing S mRNA 10 μg). Neutralizing antibody titers to the original (Washington), delta, and omicron variants of SARS-CoV-2 were measured. Mice were given a dose of 10 μg on D0, a second dose of the same on D21, and then antibody titers were measured four weeks post-second dose on D49. Cytokine levels in blood (measured via MSD) were quantified at 2, 6, 24, and 48 hours post dose after a single intramuscular dose of LPMP/SARS-CoV-2 A vaccination (containing S mRNA 10 μg).

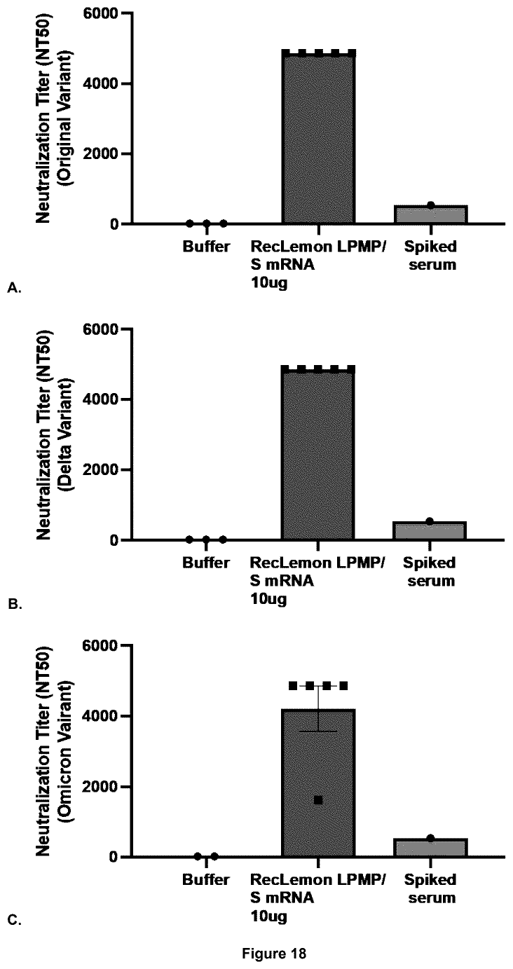

FIG. 18 shows that intramuscular vaccination of the LPMP/SARS-CoV-2 vaccine induced a robust neutralizing antibody titer in the blood of mice and was able to broadly neutralize against multiple variants of SARS-CoV2.

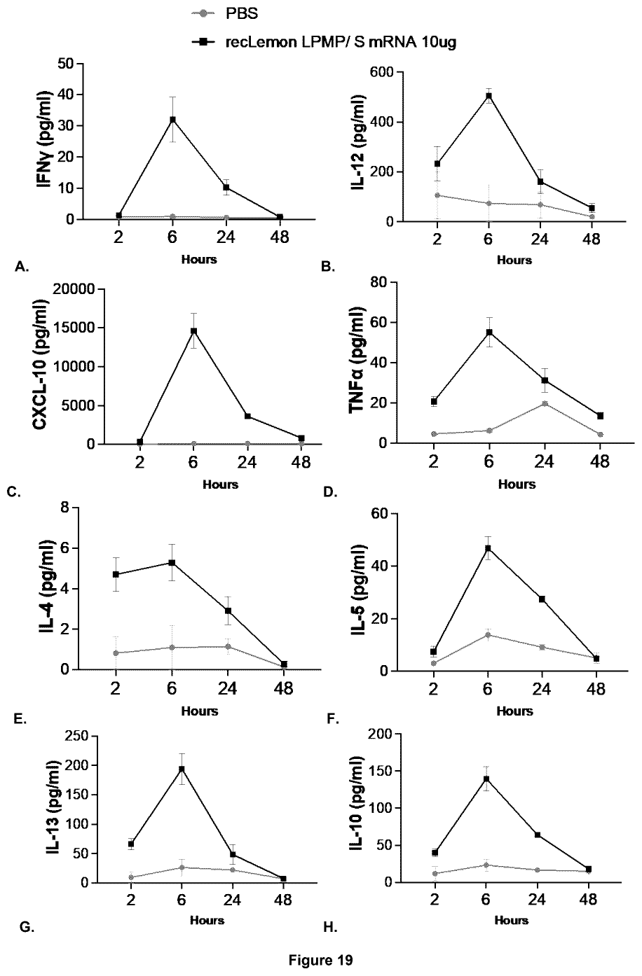

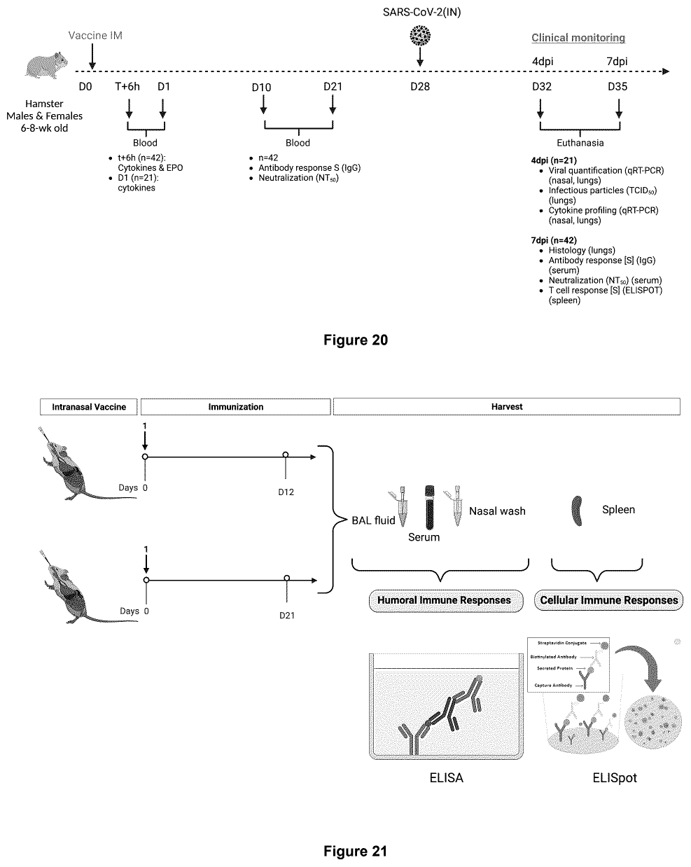

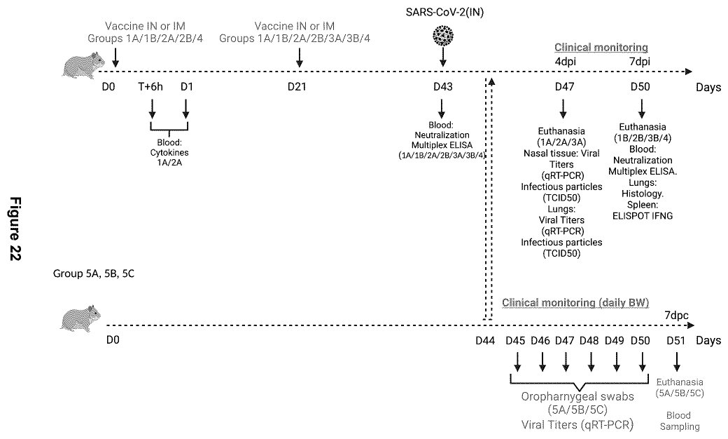

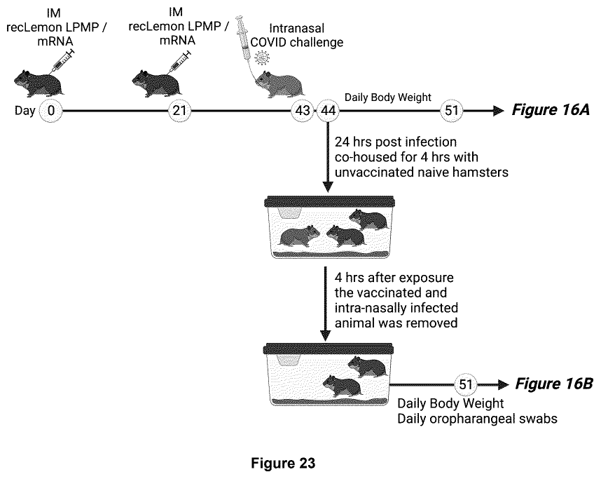

FIG. 19 shows a robust immune response through increased levels of multiple circulating cytokines and chemokines in the blood of mice at 2, 6, 24, and 48 hours, after an intramuscular vaccination of LPMP/SARS-CoV-2 A (containing S mRNA 10 μg) in the mice. Many of the increased cytokines are important for T cell responses and antibody class switching, both important aspects needed to build immunity against an infectious agent.

### Example 6. Stability of the LPMP/mRNA Formulations

The stability of the LPMP/mRNA formulations were evaluated by measuring the radiance of the mice (n=2) 4 hours after the mice were intravenously administered with a dosage of LPMP/mRNA fLuciferase (containing mRNA 5 μg). The dosing and the measurements were carried out after the LPMP/mRNA formulations (in liquid form) were stored at 4° C. over a period of time (Day 1, Day 30, Day 60).

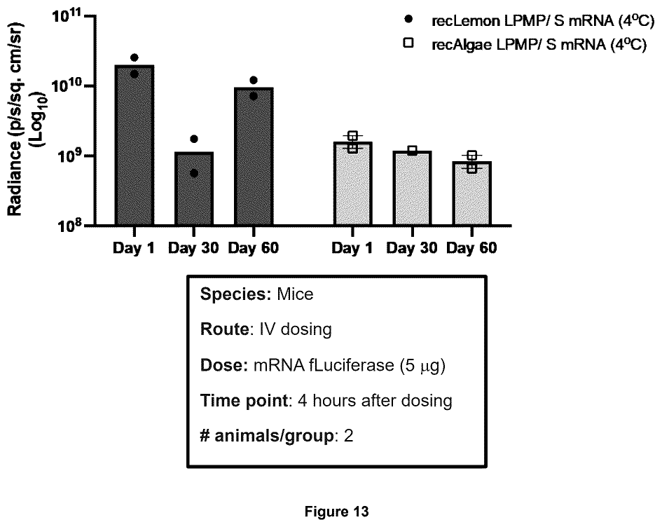

As demonstrated in FIG. 13, the LPMP/mRNA formulations were stable at 4° C. in liquid form.

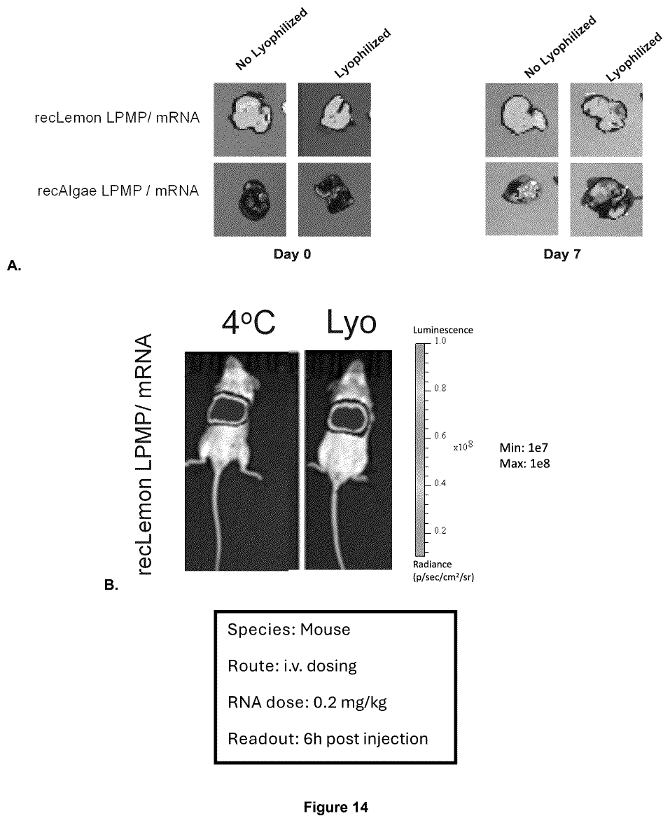

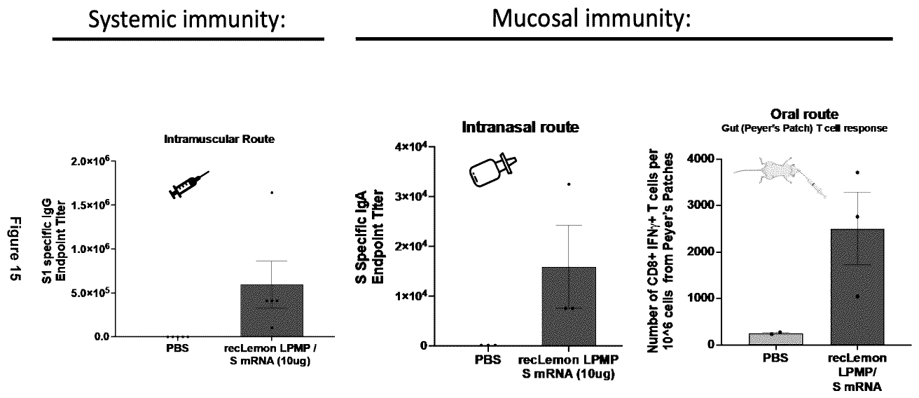

Additionally, FIGS. 14A-14B show the stability of the LPMP/mRNA formulations with lyophilization, without lyophilization, and at 4° C. over a period of time (Day 1, Day 7), with the images taken of the mouse 6 hours after the mouse was intravenously administered with a dosage of LPMP/mRNA fLuciferase (containing mRNA 0.2 mg/kg). As demonstrated in FIGS. 14A-14B, the LPMP/mRNA formulations can be lyophilized if required to achieve 4° C. stability.

### Example 7. Intranasal Vaccination of Mice with LPMP/mRNA Formulations

The LPMP/SARS-CoV-2 vaccine formulation was obtained according to Example 2.

The exemplified tested samples were reconstructed lemon (recLemon) LPMP formulation with SARS-CoV-2 (recLecom LPMP/SARS-CoV-2).

The design of single intranasal vaccination of the LPMP/SARS-CoV-2 vaccine of mice administered with the LPMP/SARS-CoV-2 vaccine is shown in Scheme 2.

To examine the efficiency of the lipid reconstructed LPMP vaccine formulation via a mucosal route, mice were dosed intranasally with LPMP/SARS-CoV-2 (mRNA encoding full length S1 glycoprotein). To examine immunogenicity of the LPMP/SARS-CoV-2 vaccine's encoding S-protein mRNA at the effector and memory phases, mice were dosed on day 0, and mice were euthanized on either day 12 or day 21, respectively. Blood, nasal washes, broncho alveolar lavage (BAL) fluid and spleens, were collected and harvested and examined for antigen specific T cells and B cells responses.

To examine the tissues for antigen-specific T cells, ELISpots were utilized. Briefly, single cell suspensions from spleens were co-cultured overnight with splenocytes pulsed with S-protein peptides, or with unpulsed splenocytes. Using ELISpots co-cultured single cell suspensions spleens were quantitatively measured for the frequency of effector cytokines (IFNg, TNFa) secreted for a single cell.

To examine antigen specific immunoglobulins in the serum, nasal washes, and BAL fluid, S1 protein and receptor binding domain (RBD) peptides coated Maxisorp plates were utilized. Briefly, Maxisorp plates were coated with either S1 protein peptide or RBD peptides overnight and IgA and total IgG were measured in serum, nasal washes, and BAL fluid using the corresponding HRP-conjugated secondary antibodies and a tetramethylbenzidine substrate.

The results are shown in FIG. 15. As shown in FIG. 15, there were immune responses against S antigen of SARS-CoV-2 in mice after administration of LPMP/SARS-CoV-2 formulation in the mice via intramuscular route (0.4 mg/kg), intranasal route (0.4 mg/kg), and oral route (8 mg/kg).

The data in these examples have demonstrated the ability of the vaccine formulations to be programmed with precise biodistribution to the lymph nodes, de-targeting the liver and, minimizing systemic exposure (vs. standard LNP/mRNA formulations which are known to target liver).

The data in these examples have also demonstrated that a single intramuscular dose of the LPMP/mRNA formulation vaccine was able to protect against SARS-CoV-2 coronavirus (COVID-19) at good benchmark levels with single dose (vs. the benchmarks using 2-dose regimen); was able to generate ˜1,000× higher antibody titers than the benchmark with ˜60% lower mRNA dose; was able to broadly neutralize against variants of SARS-CoV2 (including delta and omicron); and was able to generate systemic and mucosal immunity for prevention of SARS-CoV-2 coronavirus (COVID-19) transmission. Additionally, the examples demonstrated, by design, an improved safety profile (e.g., low reactogenicity, low levels of inflammatory or toxicity markers).

### Example 8. Prevention of Virus Transmission in Hamster Challenge Model with Intramuscular Vaccination with LPMP/mRNA Formulations

The LPMP/SARS-CoV-2 vaccine formulation was prepared according to Example 2. The exemplified tested samples were reconstructed lemon (recLemon) LPMP formulation with SARS-CoV-2 (recLecom LPMP/SARS-CoV-2).

The study design of prevention of virus transmission in a hamster challenge model with LPMP/mRNA formulations is shown in Scheme 3.

The study design of prevention of virus transmission in a hamster challenge model with 2-doses of intramuscular vaccination with the LPMP/mRNA formulations is shown in Scheme 4.

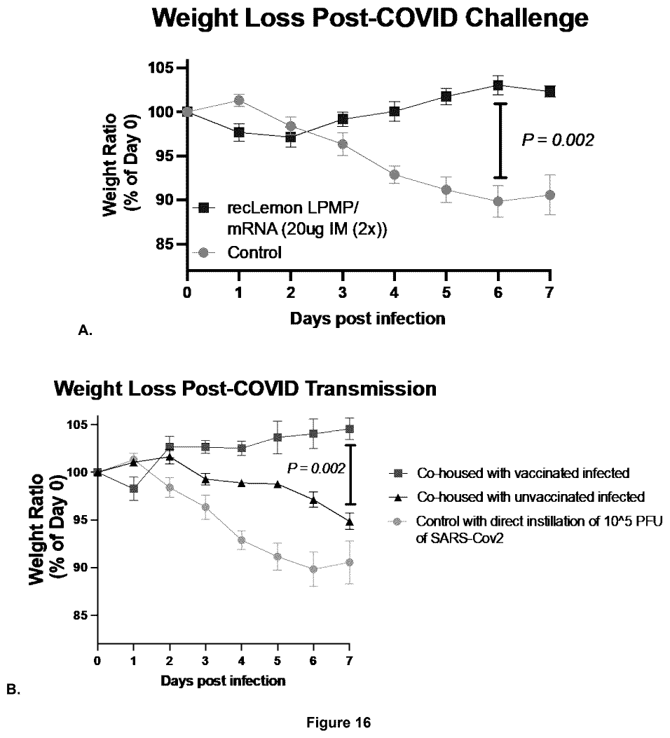

The results are shown in FIGS. 16A and 16B. Body weight is one of the hallmark readouts of hamster model for SARS-CoV-2 coronavirus (COVID-19) transmission studies. As shown in FIG. 16A, the administration of LPMP/SARS-CoV-2 formulation in the hamsters fully protected hamsters from the SARS-CoV2 infection upon challenge. As shown in FIG. 16B, the hamsters vaccinated with the LPMP/SARS-CoV-2 formulation and challenge with the SARS-CoV-2 coronavirus (COVID-19) did not transmit the virus to naïve hamsters.

Without being bound by theory, intramuscular vaccination of hamsters with the LPMP/mRNA formulations have created antibodies in nasal mucosal, which may have resulted in systemic and/or mucosal immunity for preventing virus (SARS-CoV-2 coronavirus, COVID-19) transmission.

1. A nucleic acid vaccine, comprising:
one or more polynucleotides encoding one or more antigenic polypeptides derived from an infectious agent that causes an infectious disease, disorder, or condition,
formulated within a lipid reconstructed plant messenger packs (LPMPs) comprising natural lipids and an ionizable lipid, wherein the ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

2. The nucleic acid vaccine of claim 1, wherein the natural lipids are extracted from lemon or algae.

3. The nucleic acid vaccine of claim 1, wherein the LPMPs further comprise a sterol and a polyethylene glycol (PEG)-lipid conjugate.

4. The nucleic acid vaccine of claim 3, wherein the LPMPs comprise ionizable lipid:natural lipids:sterol:PEG-lipid at a molar ratio of about 35:50:12.5:2.5 or about 35:20:42.5:2.5.

5. (canceled)

6. The nucleic acid vaccine of claim 1, wherein the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

7. (canceled)

8. The nucleic acid vaccine of claim 1, wherein the polynucleotide is mRNA.

9. The nucleic acid vaccine of claim 1, wherein the antigenic polypeptide is a corona virus, or a fragment or subunit thereof.

10. (canceled)

11. The nucleic acid vaccine of claim 1, wherein the antigenic polypeptide is a SARS virus, or a fragment or subunit thereof.

12. The nucleic acid vaccine of claim 11, wherein the antigenic polypeptide is a SARS-CoV-2 spike protein or a SARS-CoV-2 spike glycoprotein.

13-33. (canceled)

34. The nucleic acid vaccine of claim 1, wherein the LPMP has a size of less than about 100 nm.

35-37. (canceled)

38. The nucleic acid vaccine of claim 1, wherein the LPMP comprises:
about 20 mol % to about 50 mol % of the ionizable lipid,
about 20 mol % to about 60 mol % of the natural lipids,
about 7 mol % to about 20 mol % of the sterol, and
about 0.5 mol % to about 3 mol % of the polyethylene glycol (PEG)-lipid conjugate.

39. (canceled)

40. The nucleic acid vaccine of claim 1, wherein the nucleic acid vaccine has a total lipid:polynucleotide weight ratio of about 50:1 to about 10:1.

41-44. (canceled)

45. The nucleic acid vaccine of claim 1, further comprising a HEPES or TRIS buffer at a pH of about 7.0 to about 8.5.

46-47. (canceled)

48. The nucleic acid vaccine of claim 1, further comprising one or more cryoprotectants.

49-50. (canceled)

51. The nucleic acid vaccine of claim 1, wherein the nucleic acid vaccine is a lyophilized composition.

52. The nucleic acid vaccine of claim 51, wherein the lyophilized nucleic acid vaccine comprises one or more lyoprotectants.

53-63. (canceled)

64. The nucleic acid vaccine of claim 1, wherein the infectious agent is a virus.

65. The nucleic acid vaccine of claim 64, wherein the infectious agent is a virus selected from the group consisting of an influenza virus, a corona virus, a mosquito-borne virus, a hepatitis virus, and an HIV virus.

66. The nucleic acid vaccine of claim 64, wherein the infectious agent is a virus selected from the group consisting of a respiratory syncytial virus, a rhinovirus, an adenovirus, and a parainfluenza virus.

67. A method for making a nucleic acid vaccine, comprising:
reconstituting a film comprising purified PMP lipids in the presence of an ionizable lipid to produce a lipid reconstructed plant messenger packs (LPMP) comprising the ionizable lipid, wherein the ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10, and
loading into the LPMPs with one or more polynucleotides encoding one or more antigenic polypeptides derived from an infectious agent that causes an infectious disease, disorder, or condition.

68. A method of preventing or reducing the transmission of an infectious disease, disorder, or condition, comprising:
administering to a subject the nucleic acid vaccine of claim 1, thereby preventing or reducing the transmission of an infectious disease, disorder, or condition.

69. The method of claim 68, wherein the method prevents or reduces the transmission of the infectious agent from a vaccinated host to an unvaccinated host or from a vaccinated host to a vaccinated host.

70. (canceled)


SEQUENCE LISTING (7 sequences)

SEQ ID NO: 1 - RNA, 30 nt, synthetic construct
  source 1..30: mol_type = other RNA
aaaaaaaaaaaaaaaaaaaaaaaaaaaaaa

SEQ ID NO: 2 - RNA, 70 nt, synthetic construct
  source 1..70: mol_type = other RNA
aaaaaaaaaaaaaaaaaaaaaaaaaaaaaaaaaaaaaaaaaaaaaaaaaaaaaaaaaaaaaaaaaaaaaa

SEQ ID NO: 3 - RNA, 11 nt, synthetic construct
  source 1..11: mol_type = other RNA
gggatcctacc

SEQ ID NO: 4 - RNA, 250 nt, synthetic construct
  source 1..250: mol_type = other RNA
  misc_difference 1..250: note = This sequence may encompass 50-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - DNA, 22 bp, synthetic construct
  source 1..22: mol_type = other DNA
ccgcaaggttcttcttcgtaag

SEQ ID NO: 6 - DNA, 25 bp, synthetic construct
  source 1..25: mol_type = other DNA
tgctatgtttagtgttccagttttc

SEQ ID NO: 7 - RNA, 200 nt, synthetic construct
  source 1..200: mol_type = other RNA
  misc_difference 1..200: note = This sequence may encompass 100-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa